(19) United States
(12) Patent Application Publication
(10) Pub. No.: US 2014/0148459 A1
(43) Pub. Date: May 29, 2014

(54) MATRIX METALLOPROTEINASE INHIBITORS

(71) Applicant: RANBAXY LABORATORIES LIMITED, New Delhi, Delhi (IN)

(72) Inventors: Manoj Kumar Khera, Gurgaon (IN); Ajay Soni, New Delhi (IN); Jitendra Sattigeri, Gurgaon (IN); Viswajanani Sattigeri, Gurgaon (IN); Biswajit Das, Gurgaon (IN); Ian A. Cliffe, Gurgaon (IN); Pradip Kumar Bhatnagar, Exton, PA (US); Abdul Rehman Abdul Rauf, Buldana (IN); Arpita Musib, Bankura (IN); Subham Saha, Hyderabad (IN); Neeraj Kumar Yadav, Farrukhabad (IN); Sabir Ahammed, Pondicherry (IN); Ranadheer R. Reddy, Suryapet (IN); Abhijit Ray, New Delhi (IN); Punit Srivastava, Gurgaon (IN); Sunanda Ghosh Dastidar, New Delhi (IN)

(73) Assignee: RANBAXY LABORATORIES LIMITED, New Delhi, Delhi (IN)

(21) Appl. No.: 13/825,939

(22) PCT Filed: Sep. 26, 2011
(86) PCT No.: PCT/IB2011/054227

§ 371 Date: Aug. 26, 2013

(30) Foreign Application Priority Data
Sep. 24, 2010 (IN) 2280/DEL/2010

(51) Int. Cl. C07D 253/08; A61K 45/06; C07D 409/12; C07D 237/32; A61K 31/502; C07D 403/12; A61K 31/53; C07D 401/12

(52) U.S. Cl. CPC C07D 253/08; A61K 31/53; A61K 45/06; C07D 401/12; C07D 237/32; A61K 31/502; C07D 403/12; C07D 409/12
USPC 514/243; 544/183; 544/237; 514/248

(57) ABSTRACT

This invention also relates to pharmacological compositions containing the compounds of the present invention, and methods of treating asthma, rheumatoid arthritis, COPD, rhinitis, osteoarthritis, psoriatic arthritis, psoriasis, pulmonary fibrosis, pulmonary inflammation, acute respiratory distress syndrome, periodontitis, multiple sclerosis, gingivitis, gingivitis, atherosclerosis, dry eye, neointimal proliferation, which leads to restenosis and ischemic heart failure, stroke, renal diseases, tumor metastasis, and pounds.

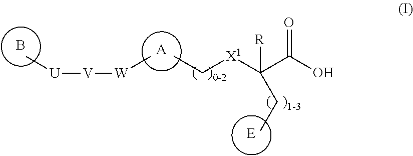

### FIELD OF THE INVENTION

The present invention relates to certain sulfonyl and oxy acetic acid derivatives and to processes for their syntheses. This invention also relates to pharmacological compositions containing the compounds of the present invention, and methods of treating asthma, rheumatoid arthritis, COPD, rhinitis, osteoarthritis, psoriatic arthritis, psoriasis, pulmonary fibrosis, pulmonary inflammation, acute respiratory distress syndrome, periodontitis, multiple sclerosis, gingivitis, atherosclerosis, dry eye, neointimal proliferation, which leads to restenosis and ischemic heart failure, stroke, renal diseases, tumor metastasis, and other inflammatory disorders characterized by over-expression and over-activation of matrix metalloproteinase using the compounds.

### BACKGROUND OF THE INVENTION

Metalloproteinases (MMPs) are a naturally occurring superfamily of proteinases (enzymes) found in most mammals. The superfamily is composed of at least 26 members of zinc-containing enzymes produced by many cell types and sharing structural and functional features. Based on structural and functional considerations, proteinases have been classified into different families and subfamilies (Vartak et al., J. Drug Targeting, 15, p. 1-20 (2007); and Hopper, FEBS, 354, p. 1-6 (1994)), such as collagenases (MMP-1, -8 and -13), gelatinases (MMP-2, and -9), metalloelastases (MMP-12), the MT-MMPs (MMP-14, -15, -16, -17, -24 and 25), matrilysins (MMP-7 and -26), stromelysins (MMP-3, -10 and -11) and sheddases such as TNF-converting enzymes (TACE, and ACE).

Metalloproteinases are believed to be important in physiological disease processes that involve remodelling such as embryonic development, bone formation and uterine remodelling during menstruation. One major biological function of MMPs is to catalyze the breakdown of connective tissues or extra-cellular matrix by their ability to hydrolyze various components of tissue or matrix. Apart from their role in degrading connective tissue, MMPs are involved in the activation of zymogen (pro) forms of other MMPs thereby inducing MMP activation. They are also involved in the biosynthesis of TNF-alpha which is implicated in many pathological conditions.

MMP-12, also known as macrophage elastase or metalloelastase, is expressed in activated macrophages and has been shown to be secreted from alveolar macrophages from smokers as well as in foam cells in atherosclerotic lesions. MMP-12 knockout mouse studies have shown the development of significant emphysema, thus supporting its role in COPD. MMP-9 (gelatinase B, 92 kDa type IV collagenase) is one member of the MMP family that is released as a proenzyme and subsequently activated via a protease cascade in vivo.

The concentration of MMP-9 is increased in diseases like asthma, interstitial pulmonary fibrosis (IPF), adult respiratory distress syndrome (ARDS), and in chronic obstructive pulmonary disease (COPD). Because of its proteolytic ability, MMP-9 has been implicated in tissue remodelling of the airways and lungs in chronic inflammatory diseases such as severe asthma and COPD. MMP-9 is also likely to be physiologically important because of its ability to regulate the digestion of components of the extracellular matrix as well as the activity of other proteases and cytokines. MMP-9 is secreted in neutrophils, macrophages, osteoclasts, which are easily induced by cytokines and growth factors, and plays a role in various physiological and pathological processes.

Over-expression or over-activation of an MMP, or an imbalance between an MMP and a natural (i.e., endogenous) tissue inhibitor of a matrix metalloproteinase (TIMP) has been linked to a pathogenesis of diseases characterized by the breakdown of connective tissue or extracellular matrix.

Inhibition of the activity of one or more MMPs may be of benefit in treatment of various inflammatory, autoimmune and allergic diseases such as inflammation of the joint, inflammation of the GI tract, inflammation of the skin, collagen remodeling, wound healing disorders, etc.

The design and therapeutic application of MMP inhibitors has revealed that the requirement of a molecule to be an effective inhibitor of MMP class of enzymes is a functional group (e.g. carboxylic acid, hydroxamic acid or sulphydryl) capable of chelating to the active site Zn²⁺ ion (Whittaker et al., Chem. Rev., 99 p. 2735-76 (1999)).

WO 2004/046119 discloses substituted aralkyl derivatives that are useful as antidiabetic, hypolipidaemic and hypocholesterolemic agents. EP 0 364 804 discloses compounds that are non-peptide rennin inhibitors. U.S. Pat. No. 4,833,161 discloses carboxylic acid derivatives that are useful for the treatment of diabetes, adipositas or atherosclerosis. WO 2004/096764 relates to a method of preparing a chiral compound having a stereogenic carbon atom adjacent to a nonstereogenic quaternary carbon atom bearing diastereotopic groups. WO 03/008380 relates to novel compound having α2β1 integrin inhibitory activity. WO 2004/110974 discloses compounds and their physiologically functional derivatives described as inhibitors of matrix metalloproteinase enzymes. WO 2004/113279 discloses alleged inhibitors of matrix metalloproteinase. WO 2005/026120 discloses compounds also described as inhibitors of matrix metalloproteinase. U.S. Patent Application No. 2003/0139453 discloses difluorobutyric acid compounds useful for treating diseases associated with zinc metalloprotease activity. WO 2006/090235 describes 5-phenyl-pentanoic acid derivatives described as matrix metalloproteinase inhibitors for the treatment of asthma and other diseases.

Research has been carried out into the identification of inhibitors that are selective, e.g., for a few of the MMP subtypes. An MMP inhibitor of improved selectivity would avoid potential side effects associated with inhibition of MMPs that are not involved in the pathogenesis of the disease being treated.

Further, use of more selective MMP inhibitors would require administration of a lower amount of the inhibitor for treatment of disease than would otherwise be required and, after administration, partitioned in vivo among multiple MMPs. Still further, the administration of a lower amount of compound would improve the margin of safety between the dose of the inhibitor required for therapeutic activity and the dose of the inhibitor at which toxicity is observed.

Many drugs exist as asymmetric three-dimensional molecules, i.e., chiral, and will therefore have several stereoisomers depending upon the number of chiral centers present. The importance of evaluating new chemical entities having chiral centers as single isomers is to understand their effect on pharmacological and toxicological aspects. There are often pharmacodynamic, pharmacokinetic and/or toxicological differences between enantiomers/diastereomers. Even if natural physiological mediators are achiral, based on their target environment, their receptors/enzymes may demonstrate a preference for only one optically pure enantiomer of agonists, antagonists or inhibitors. From a pharmacokinetic point of view, chirality can have an influence on drug absorption, distribution, metabolism and elimination. Pure single isomers may also offer advantages in terms of these pharmacokinetic parameters thus enabling better developability of such molecules as drug candidates. It is also known that chirality has a significant effect of the physicochemical properties and crystallinity of a chiral molecule which in turn have profound effects on the pharmacokinetics and developability of the molecule. Besides those mentioned above, regulatory principles guide one to preferably develop single isomers as drug candidates in order to avoid any pharmacological, pharmacokinetic and toxicological problems that may arise due to interactions of an unwanted isomer with undesirable molecular targets.

In this context, synthetic strategies to produce pure single isomers offer advantages over analytical techniques of separation of isomer not only in terms of cost and efficiency but larger amounts of compound can be prepared for elaborate pharmaceutical testing. Thus, compounds of present invention, which are single chiral isomers, have improved potency, improved pharmacokinetics and/or improved physicochemical properties as compared to racemic compounds.

The present invention is directed to overcoming problems encountered in the art.

### SUMMARY OF THE INVENTION

The present invention provides some sulfonyl or oxy acetic acid derivatives which act as matrix metalloprotease inhibitors, corresponding processes for their synthesis of and pharmaceutical compositions containing the compounds of the present invention. The present invention relates to matrix metalloproteinase inhibitors useful as effective therapeutic or prophylactic agents in treatment of various inflammatory, autoimmune, and allergic diseases and other inflammatory disorders characterized by the over-expression and over-activation of a matrix metalloproteinase using the compounds.

The present invention discloses a novel class of compounds that are dual MMP-9/12 inhibitors and have desirable activity profiles. The compounds of this invention have beneficial potency and/or selectivity.

Pharmaceutical compositions containing such compounds are provided together with the pharmaceutically acceptable carriers or diluents, which can be used for the treatment or prevention of inflammatory or autoimmune diseases. These pharmaceutical compositions may be administered or co-administered by a wide variety of routes including, for example, oral, topical, rectal, intranasal or by parenteral route. The composition may also be administered or co-administered in slow release dosage forms.

Although the specific enantiomers have been shown by way of examples, the racemates, diastereomers, N-oxides, polymorphs, pharmaceutically acceptable salts, pharmaceutically acceptable solvates, co-crystals, prodrugs and metabolites having the same type of activity, are also provided. The pharmaceutical compositions comprising the compounds, their metabolites, racemates, enantiomers, N-oxides, polymorphs, solvates, co-crystals, prodrugs or pharmaceutically acceptable salts thereof, in combination with a pharmaceutically acceptable carrier and optionally included excipients are also included.

The therapeutically effective amounts of one or more compounds of the present invention can be used in combination with one or more other therapeutic agents, for example, other anti-inflammatory agents, beta agonists, antihypertensive agents, immunosuppressive agents and anti-infective agents.

Other objects will be set forth in accompanying description and in the part will be apparent from the description or may be learnt by the practice of the invention.

### DETAIL DESCRIPTION OF THE INVENTION

In accordance with one aspect, there are provided compounds having the structure

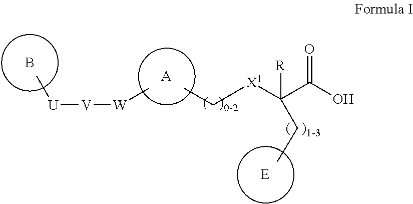

including racemates, enantiomers and diastereomers thereof, or a pharmaceutically acceptable salt thereof wherein,

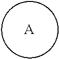

represents (un)substituted aryl or heteroaryl;

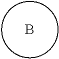

represents C₆-C₁₂ aryl, C₃-C₁₂ cycloalkyl, C₆-C₁₂ heteroaryl or C₆-C₁₂ heterocyclyl each of which is optionally further substituted by one or more substituents independently selected from R¹;
U represents bond, —NH—, —C(═O)—, —(CH₂)ₙ—, —C(═S)—, —O—, —SO₂— or —S— wherein n represents zero or an integer between 1 and 2;
V represents bond, —NH—, —C(═O)—, —C(═S)— or —SO₂—;
W represents bond, —NH—, —C(═O)—,(CH₂)ₙ—, —C(═S)—, —O—, —S— or —SO₂—;
X¹ represents —O—, —S—, —SO— or —SO₂—;
R represents H, alkyl or arylalkyl;
R¹ represents alkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙ NHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl, n is as defined earlier, and m is an integer 0-2};

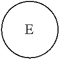

represents mono-, bi- or polycyclic heteroaryl or heterocyclyl selected from the following:

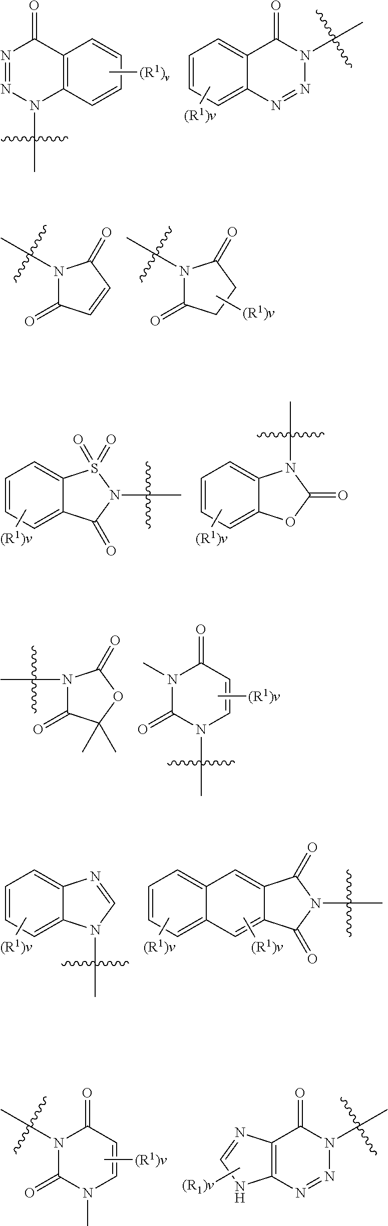

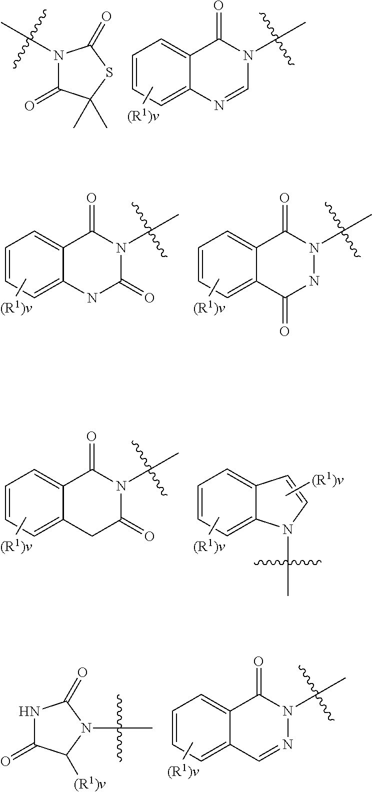

wherein R¹ is as defined earlier and v represents zero or an integer between 1-4.

In accordance with one aspect, there are provided compounds having the structure of Formula Ia;

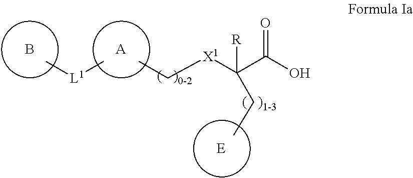

including racemates, enantiomers and diastereomers thereof, or a pharmaceutically acceptable salt thereof wherein,

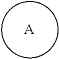

represents (un)substituted aryl or heteroaryl;

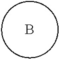

represents C₆-C₁₂ aryl, C₃-C₁₂ cycloalkyl, C₆-C₁₂ heteroaryl or C₆-C₁₂ heterocyclyl, each of which is optionally further substituted by one or more substituents independently selected from R¹;
L¹ represents bond, —(CH₂)ₙ—, —NHC(═O)(CH₂)ₙ—, —(CH₂)ₙC(═O)NH—, —NHC(═O)NH—, —SO₂NH—, —NHSO₂, —SO₂—, —NHC(═O)(O)—, —O—(CH₂)ₙ—, —(CH₂)ₙ—O—, —(CH₂)ₙOC(═O)NH—, —C(═S)NH—, —NHC(═S)— or —NHC(═S)NH— wherein n can be zero or an integer between 1 and 2;
X¹ represents —O—, —S—, —SO— or —SO₂—;
R represents H, alkyl or arylalkyl;
R¹ represents alkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)—O—C(═O)—NR_{f}R_{q}, (CH₂)ₙ NHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} independently represent hydrogen, alkyl, alkenyl, cycloalkyl aryl, heteroaryl, heterocyclyl, alkylaryl, alkylheteroaryl and alkylheterocyclyl, n is as defined earlier and m is an integer 0-2};

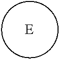

represents mono-, bi- or polycyclic heteroaryl or heterocyclyl selected from the following:

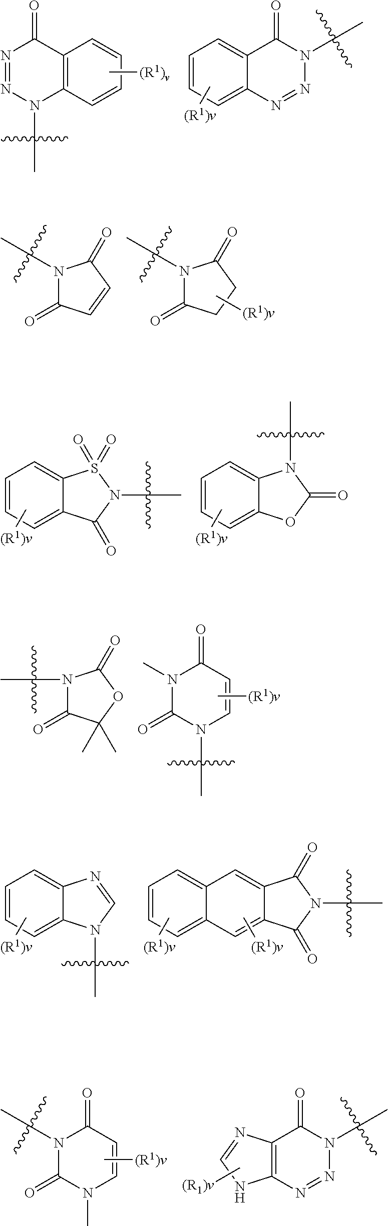

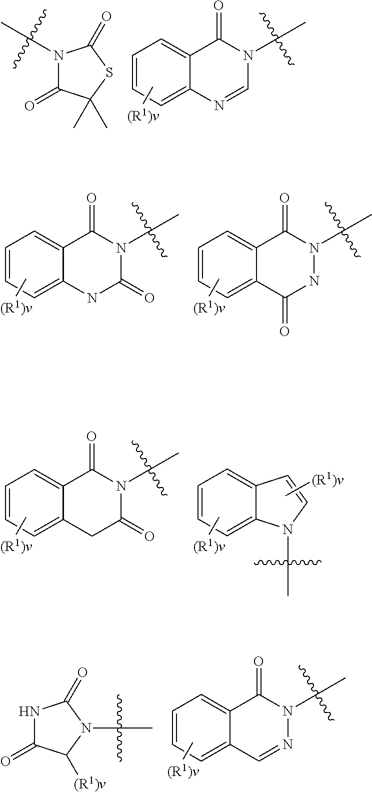

wherein R¹ is as defined earlier and v represents zero or an integer between 1-4.

The enantiomers, diastereomers, rotational isomers, N-oxides, polymorphs, pharmaceutically acceptable salts and pharmaceutically acceptable solvates of these compounds, prodrugs and metabolites having the same type of activity are also provided, as well as pharmaceutical compositions comprising the compounds, their metabolites, enantiomers, diastereomers, conformational isomers, N-oxides, polymorphs, solvates or pharmaceutically acceptable salts thereof, in combination with a pharmaceutically acceptable carrier and optionally included excipients.

In one embodiment, the invention encompasses compounds of Formula I/Ia, which may include, but are not limited to the following, for example:
2-[(4-{[(4-Methylphenyl)carbonyl]amino}phenyl)sulfonyl]-4-(4-oxo-1,2,3-benzotriazin-3(4H)-yl)butanoic acid (Compound no. 1),
2-[(3′-Methoxybiphenyl-4-yl) sulfonyl]-4-(4-oxo-1,2,3-benzotriazin-3(4H)-yl)butanoic acid (Compound no. 2),
2-[(3′-Fluoro-4′-methoxybiphenyl-4-yl)sulfonyl]-4-(6-methyl-4-oxo-1,2,3-benzotriazin-3(4H)-yl)butanoic acid (Compound no. 3),
2-{[2-(4′-Chlorobiphenyl-4-yl)ethyl]sulfonyl}-4-(4-oxo-1,2,3-benzotriazin-3(4H)-yl)butanoic acid (Compound no. 4),
2-{[2-(4′-Chlorobiphenyl-4-yl)ethyl]sulfonyl}-4-(6-fluoro-4-oxo-1,2,3-benzotriazin-3(4H)-yl)butanoic acid (Compound no. 5),
2-{[(4′-Chlorobiphenyl-4-yl)methyl]sulfonyl}-4-(7-methyl-4-oxo-1,2,3-benzotriazin-3(4H)-yl)butanoic acid (Compound no. 6),
2-{[(4′-Chlorobiphenyl-4-yl)methyl]sulfonyl}-4-(4-oxo-1,2,3-benzotriazin-3(4H)-yl)butanoic acid (Compound no. 7),
2-[(3′,4′-Difluorobiphenyl-4-yl)sulfonyl]-4-(6-methyl-4-oxo-1,2,3-benzotriazin-3(4H)-yl)butanoic acid (Compound no. 8),
2-{[(4′-Chlorobiphenyl-4-yl)methyl]sulfonyl}-4-[4-oxo-7-(trifluoromethyl)-1,2,3-benzotriazin-3(4H)-yl]butanoic acid (Compound no. 9),
4-(6-Methyl-4-oxo-1,2,3-benzotriazin-3(4H)-yl)-2-{[4′-(trifluoromethyl)biphenyl-4-yl]sulfonyl}butanoic acid (Compound no. 10),
2-[(3′,4′-Dichlorobiphenyl-4-yl)sulfonyl]-4-(6-methyl-4-oxo-1,2,3-benzotriazin-3(4H)-yl)butanoic acid (Compound no. 11),
2-[(4′-Methoxy-3′-methylbiphenyl-4-yl)sulfonyl]-4-(6-methyl-4-oxo-1,2,3-benzotriazin-3(4H)-yl)butanoic acid (Compound no. 12),
2-[(3′,4′-Difluorobiphenyl-4-yl)sulfonyl]-4-(6-methyl-4-oxo-1,2,3-benzotriazin-3(4H)-yl)butanoic acid (Compound no. 13),
2-[(3′-Fluoro-4′-methylbiphenyl-4-yl)sulfonyl]-4-(6-methyl-4-oxo-1,2,3-benzotriazin-3(4H)-yl)butanoic acid (Compound no. 14),
2-[(4′-Fluorobiphenyl-4-yl)sulfonyl]-4-(6-methyl-4-oxo-1,2,3-benzotriazin-3(4H)-yl)butanoic acid (Compound no. 15),
2-[(4′-Chlorobiphenyl-4-yl)sulfonyl]-4-(6-methyl-4-oxo-1,2,3-benzotriazin-3(4H)-yl)butanoic acid (Compound no. 16),
2-[(4′-Ethylbiphenyl-4-yl)sulfonyl]-4-(6-methyl-4-oxo-1,2,3-benzotriazin-3(4H)-yl)butanoic acid (Compound no. 17),
2-[(4′-Methoxybiphenyl-4-yl)sulfonyl]-4-(6-methyl-4-oxo-1,2,3-benzotriazin-3(4H)-yl)butanoic acid (Compound no. 18),
2-[(4′-Fluoro-3′-methylbiphenyl-4-yl)sulfonyl]-4-(6-methyl-4-oxo-1,2,3-benzotriazin-3(4H)-yl)butanoic acid (Compound no. 19),
2-{[4-(6-Methoxypyridin-3-yl)benzyl]sulfonyl}-4-(4-oxo-1,2,3-benzotriazin-3(4H)-yl)butanoic acid (Compound no. 20),
4-(4-Oxo-1,2,3-benzotriazin-3(4H)-yl)-2-({[4′-(trifluoromethoxy)biphenyl-4-yl]methyl}sulfonyl)butanoic acid (Compound no. 21),
2-{[(3′,4′-Dimethoxybiphenyl-4-yl)methyl]sulfonyl}-4-(4-oxo-1,2,3-benzotriazin-3(4H)-yl)butanoic acid (Compound no. 22),
2-{[(3′-Fluoro-4′-methylbiphenyl-4-yl)methyl]sulfonyl}-4-(4-oxo-1,2,3-benzotriazin-3(4H)-yl)butanoic acid (Compound no. 23),
2-{[(3′,4′-Dimethylbiphenyl-4-yl)methyl]sulfonyl}-4-(4-oxo-1,2,3-benzotriazin-3(4H)-yl)butanoic acid (Compound no. 24),
2-{[(3′,4′-Dichlorobiphenyl-4-yl)methyl]sulfonyl}-4-(4-oxo-1,2,3-benzotriazin-3(4H)-yl)butanoic acid (Compound no. 25),
2-{[(4′-Fluoro-3′-methylbiphenyl-4-yl)methyl]sulfonyl}-4-(4-oxo-1,2,3-benzotriazin-3(4H)-yl)butanoic acid (Compound no. 26),
4-(4-Oxo-1,2,3-benzotriazin-3(4H)-yl)-2-({[4′-(trifluoromethyl)biphenyl-4-yl]methyl}sulfonyl)butanoic acid (Compound no. 27),
2-{[(4′-Methoxybiphenyl-4-yl)methyl]sulfonyl}-4-(4-oxo-1,2,3-benzotriazin-3(4H)-yl)butanoic acid (Compound no. 28),
2-{[(4′-Fluorobiphenyl-4-yl)methyl]sulfonyl}-4-(4-oxo-1,2,3-benzotriazin-3(4H)-yl)butanoic acid (Compound no. 29),
2-({2-[4-(6-Methoxypyridin-3-yl)phenyl]ethyl}sulfonyl)-4-(4-oxo-1,2,3-benzotriazin-3(4H)-yl)butanoic acid (Compound no. 30),
2-{[2-(3′-Fluoro-4′-methylbiphenyl-4-yl)ethyl]sulfonyl}-4-(4-oxo-1,2,3-benzotriazin-3(4H)-yl)butanoic acid (Compound no. 31),
2-{[2-(4′-Ethylbiphenyl-4-yl)ethyl]sulfonyl}-4-(4-oxo-1,2,3-benzotriazin-3(4H)-yl)butanoic acid (Compound no. 32),
2-{[2-(3′,4′-Difluorobiphenyl-4-yl)ethyl]sulfonyl}-4-(4-oxo-1,2,3-benzotriazin-3(4H)-yl)butanoic acid (Compound no. 33),
2-{[2-(4′-Cyanobiphenyl-4-yl)ethyl]sulfonyl}-4-(4-oxo-1,2,3-benzotriazin-3(4H)-yl)butanoic acid (Compound no. 34),
2-{[2-(3′-Fluoro-4′-methoxybiphenyl-4-yl)ethyl]sulfonyl}-4-(4-oxo-1,2,3-benzotriazin-3(4H)-yl)butanoic acid (Compound no. 35),
2-({2-[4-(1-Methyl-1H-pyrazol-4-yl)phenyl]ethyl}sulfonyl)-4-(4-oxo-1,2,3-benzotriazin-3(4H)-yl)butanoic acid (Compound no. 36),
2-{[2-(4′-Methylbiphenyl-4-yl)ethyl]sulfonyl}-4-(4-oxo-1,2,3-benzotriazin-3(4H)-yl)butanoic acid (Compound no. 37),
4-(4-Oxo-1,2,3-benzotriazin-3(4H)-yl)-2-({2-[4′-(trifluoromethoxy)biphenyl-4-yl]ethyl}sulfonyl)butanoic acid (Compound no. 38),
4-(7-Methyl-4-oxo-1,2,3-benzotriazin-3(4H)-yl)-2-{[4′-(trifluoromethoxy)biphenyl-4-yl]sulfonyl}butanoic acid (Compound no. 39),
4-(6-Methoxy-4-oxo-1,2,3-benzotriazin-3(4H)-yl)-2-{[4′-(trifluoromethoxy)biphenyl-4-yl]sulfonyl}butanoic acid (Compound no. 40),
4-(7-Methoxy-4-oxo-1,2,3-benzotriazin-3(4H)-yl)-2-{[4′-(trifluoromethoxy)biphenyl-4-yl]sulfonyl}butanoic acid (Compound no. 41),
2-[(4′-tert-Butylbiphenyl-4-yl)sulfonyl]-4-(4-oxo-1,2,3-benzotriazin-3(4H)-yl)butanoic acid (Compound no. 42),
2-[(4′-Ethylbiphenyl-4-yl)sulfonyl]-4-(4-oxo-1,2,3-benzotriazin-3(4H)-yl)butanoic acid (Compound no. 43),
4-(4-Oxo-1,2,3-benzotriazin-3(4H)-yl)-2-{[4′-(propan-2-yl)biphenyl-4-yl]sulfonyl}butanoic acid (Compound no. 44),
4-(4-Oxo-1,2,3-benzotriazin-3(4H)-yl)-2-{[4′-(trifluoromethoxy)biphenyl-4-yl]sulfonyl}butanoic acid (Compound no. 45),
4-(4-Oxo-1,2,3-benzotriazin-3 (4H)-yl)-2-({4-[(phenylcarbonyl)amino]phenyl}sulfonyl)butanoic acid (Compound no. 46),
2-[(4-{[(4-Methoxyphenyl)carbonyl]amino}phenyl)sulfonyl]-4-(4-oxo-1,2,3-benzotriazin-3(4H)-yl)butanoic acid (Compound no. 47),
4-(7-Methoxy-4-oxo-1,2,3-benzotriazin-3(4H)-yl)-2-{[4′-(trifluoromethyl)biphenyl-4-yl]sulfonyl}butanoic acid (Compound no. 48),
2-[(4′-Methoxybiphenyl-4-yl)sulfonyl]-4-(7-methoxy-4-oxo-1,2,3-benzotriazin-3(4H)-yl)butanoic acid (Compound no. 49),
2-[(3′-Fluoro-4′-methylbiphenyl-4-yl)sulfonyl]-4-(7-methoxy-4-oxo-1,2,3-benzotriazin-3(4H)-yl)butanoic acid (Compound no. 50),
2-[(3′,4′-Dimethoxybiphenyl-4-yl)sulfonyl]-4-(7-methoxy-4-oxo-1,2,3-benzotriazin-3(4H)-yl)butanoic acid (Compound no. 51),
2-[(4-{[(3-Methoxyphenyl)carbonyl]amino}phenyl)sulfonyl]-4-(4-oxo-1,2,3-benzotriazin-3(4H)-yl)butanoic acid (Compound no. 52),
2-[(4-{[(3-Fluorophenyl)carbonyl]amino}phenyl)sulfonyl]-4-(4-oxo-1,2,3-benzotriazin-3(4H)-yl)butanoic acid (Compound no. 53),
2-[(4-{[(4-Fluorophenyl)carbonyl]amino}phenyl)sulfonyl]-4-(4-oxo-1,2,3-benzotriazin-3(4H)-yl)butanoic acid (Compound no. 54),
2-[(4-{[(4-Chlorophenyl)carbonyl]amino}phenyl)sulfonyl]-4-(4-oxo-1,2,3-benzotriazin-3(4H)-yl)butanoic acid (Compound no. 55),
2-[(4′-Ethylbiphenyl-4-yl)sulfonyl]-4-(7-methoxy-4-oxo-1,2,3-benzotriazin-3(4H)-yl)butanoic acid (Compound no. 56),
2-[(4′-Chlorobiphenyl-4-yl)sulfonyl]-4-(7-methoxy-4-oxo-1,2,3-benzotriazin-3(4H)-yl)butanoic acid (Compound no. 57),
2-[(3′,4′-Dichlorobiphenyl-4-yl)sulfonyl]-4-(7-methoxy-4-oxo-1,2,3-benzotriazin-3(4H)-yl)butanoic acid (Compound no. 58),
2-[(3′-Fluoro-4′-methoxybiphenyl-4-yl)sulfonyl]-4-(7-methoxy-4-oxo-1,2,3-benzotriazin-3(4H)-yl)butanoic acid (Compound no. 59),
2-[(3′,4′-Difluorobiphenyl-4-yl)sulfonyl]-4-(6-methoxy-4-oxo-1,2,3-benzotriazin-3(4H)-yl)butanoic acid (Compound no. 60),
2-[(3′-Fluoro-4′-methylbiphenyl-4-yl)sulfonyl]-4-(6-methoxy-4-oxo-1,2,3-benzotriazin-3(4H)-yl)butanoic acid (Compound no. 61),
2-[(4′-Fluoro-3′-methylbiphenyl-4-yl)sulfonyl]-4-(6-methoxy-4-oxo-1,2,3-benzotriazin-3(4H)-yl)butanoic acid (Compound no. 62),
2-[(3′,4′-Dichlorobiphenyl-4-yl)sulfonyl]-4-(6-methoxy-4-oxo-1,2,3-benzotriazin-3(4H)-yl)butanoic acid (Compound no. 63),
2-[(3′-Fluoro-4′-methoxybiphenyl-4-yl)sulfonyl]-4-(6-methoxy-4-oxo-1,2,3-benzotriazin-3(4H)-yl)butanoic acid (Compound no. 64),
2-{[4′-Chloro-3′-(trifluoromethyl)biphenyl-4-yl]sulfonyl}-4-(6-methoxy-4-oxo-1,2,3-benzotriazin-3(4H)-yl)butanoic acid (Compound no. 65),
2-[(4′-Ethylbiphenyl-4-yl)sulfonyl]-4-(6-methoxy-4-oxo-1,2,3-benzotriazin-3(4H)-yl)butanoic acid (Compound no. 66),
2-[(3′,4′-Dimethylbiphenyl-4-yl)sulfonyl]-4-(6-methoxy-4-oxo-1,2,3-benzotriazin-3(4H)-yl)butanoic acid (Compound no. 67),
4-(6-Methoxy-4-oxo-1,2,3-benzotriazin-3(4H)-yl)-2-[(4′-methylbiphenyl-4-yl)sulfonyl]butanoic acid (Compound no. 68),
2-[(4′-Chlorobiphenyl-4-yl)sulfonyl]-4-(6-methoxy-4-oxo-1,2,3-benzotriazin-3(4H)-yl)butanoic acid (Compound no. 69),
4-(6-Methoxy-4-oxo-1,2,3-benzotriazin-3(4H)-yl)-2-{[4′-(trifluoromethyl)biphenyl-4-yl]sulfonyl}butanoic acid (Compound no. 70),
2-[(4′-Methoxybiphenyl-4-yl)sulfonyl]-4-(6-methoxy-4-oxo-1,2,3-benzotriazin-3(4H)-yl)butanoic acid (Compound no. 71),
4-(4-Oxo-1,2,3-benzotriazin-3(4H)-yl)-2-({2-[4-({[4-(trifluoromethyl)phenyl]carbonyl}amino)phenyl]ethyl}sulfonyl)butanoic acid (Compound no. 72),
4-(4-Oxo-1,2,3-benzotriazin-3(4H)-yl)-2-({2-[4-({[4-(trifluoromethoxy)phenyl]carbonyl}amino)phenyl]ethyl}sulfonyl)butanoic acid (Compound no. 73),
2-[(4-{[(3,4-Dichlorophenyl)carbonyl]amino}phenyl)sulfonyl]-4-(4-oxo-1,2,3-benzotriazin-3(4H)-yl)butanoic acid (Compound no. 74),
2-[(3′-Fluoro-4′-methoxybiphenyl-4-yl)sulfanyl]-4-[4-oxo-7-(trifluoromethyl)-1,2,3-benzotriazin-3(4H)-yl]butanoic acid (Compound no. 75),
4-(4-Oxo-1,2,3-benzotriazin-3(4H)-yl)-2-[(2-{4-[(phenylcarbonyl)amino]phenyl}ethyl)sulfonyl]butanoic acid (Compound no. 76),
4-(4-Oxo-1,2,3-benzotriazin-3(4H)-yl)-2-[(2-{4-[(thiophen-2-ylcarbonyl)amino]phenyl}ethyl)sulfonyl]butanoic acid (Compound no. 77),
2-[(2-{4-[(Cyclopentylcarbonyl)amino]phenyl}ethyl)sulfonyl]-4-(4-oxo-1,2,3-benzotriazin-3(4H)-yl)butanoic acid (Compound no. 78),
2-[(2-{4-[(Cyclopropylcarbonyl)amino]phenyl}ethyl)sulfonyl]-4-(4-oxo-1,2,3-benzotriazin-3(4H)-yl)butanoic acid (Compound no. 79),
2-{[2-(4-{[(3-Methoxyphenyl)carbonyl]amino}phenyl)ethyl]sulfonyl}-4-(4-oxo-1,2,3-benzotriazin-3(4H)-yl)butanoic acid (Compound no. 80),
2-{[2-(4-{[(3-Chlorophenyl)carbonyl]amino}phenyl)ethyl]sulfonyl}-4-(4-oxo-1,2,3-benzotriazin-3(4H)-yl)butanoic acid (Compound no. 81),
2-{[2-(4-{[(3-Fluorophenyl)carbonyl]amino}phenyl)ethyl]sulfonyl}-4-(4-oxo-1,2,3-benzotriazin-3(4H)-yl)butanoic acid (Compound no. 82),
2-{[2-(4-{[(4-Ethylphenyl)carbonyl]amino}phenyl)ethyl]sulfonyl}-4-(4-oxo-1,2,3-benzotriazin-3(4H)-yl)butanoic acid (Compound no. 83),
2-{[2-(4-{[(4-Methoxyphenyl)sulfonyl]amino}phenyl)ethyl]sulfonyl}-4-(4-oxo-1,2,3-benzotriazin-3(4H)-yl)butanoic acid (Compound no. 84),
2-[(4-{[(3-Ethoxyphenyl)carbamoyl]amino}phenyl)sulfonyl]-4-(4-oxo-1,2,3-benzotriazin-3(4H)-yl)butanoic acid (Compound no. 85),
2-{[4-({[2-Fluoro-5-(trifluoromethyl)phenyl]carbamoyl}amino)phenyl]sulfonyl}-4-(4-oxo-1,2,3-benzotriazin-3(4H)-yl)butanoic acid (Compound no. 86),
2-[(4-{[(2,6-Dimethoxyphenyl)carbonyl]amino}phenyl)sulfonyl]-4-(4-oxo-1,2,3-benzotriazin-3(4H)-yl)butanoic acid (Compound no. 87),
2-[(4-{[(4-Fluorophenyl)carbamoyl]amino}phenyl)sulfonyl]-4-(4-oxo-1,2,3-benzotriazin-3(4H)-yl)butanoic acid (Compound no. 88),
2-[(4′-Fluorobiphenyl-4-yl)sulfonyl]-4-(8-methyl-4-oxo-1,2,3-benzotriazin-3(4H)-yl)butanoic acid (Compound no. 89),
2-[(3′-Ffluoro-4′-methylbiphenyl-4-yl)sulfonyl]-4-(8-methyl-4-oxo-1,2,3-benzotriazin-3(4H)-yl)butanoic acid (Compound no. 90),
2-[(4′-Fluoro-3′-methylbiphenyl-4-yl)sulfonyl]-4-(8-methyl-4-oxo-1,2,3-benzotriazin-3(4H)-yl)butanoic acid (Compound no. 91),
2-({2-[4-(Benzyloxy)phenyl]ethyl}sulfonyl)-4-(4-oxo-1,2,3-benzotriazin-3(4H)-yl)butanoic acid (Compound no. 92),
2-[(3′,4′-Dimethylbiphenyl-4-yl)sulfonyl]-4-(8-methyl-4-oxo-1,2,3-benzotriazin-3(4H)-yl)butanoic acid (Compound no. 93),
2-[(4-{[(3-Methylphenyl)carbonyl]amino}phenyl)sulfonyl]-4-(4-oxo-1,2,3-benzotriazin-3(4H)-yl)butanoic acid (Compound no. 94),
2-[(4-{[(2-Methoxyphenyl)carbonyl]amino}phenyl)sulfonyl]-4-(4-oxo-1,2,3-benzotriazin-3(4H)-yl)butanoic acid (Compound no. 95),
2-[(4′-Ethylbiphenyl-4-yl)sulfonyl]-4-(8-methyl-4-oxo-1,2,3-benzotriazin-3(4H)-yl)butanoic acid (Compound no. 96),
2-[(4′-Methylbiphenyl-4-yl)sulfonyl]-4-(8-methyl-4-oxo-1,2,3-benzotriazin-3(4H)-yl)butanoic acid (Compound no. 97),
2-{[4-(6-Methoxypyridin-3-yl)phenyl]sulfonyl}-4-(8-methyl-4-oxo-1,2,3-benzotriazin-3(4H)-yl)butanoic acid (Compound no. 98),
2-({4-[(Cyclohexylcarbonyl)amino]phenyl}sulfonyl)-4-(4-oxo-1,2,3-benzotriazin-3(4H)-yl)butanoic acid (Compound no. 99),
2-[(4-{[(2-Methylphenyl)carbonyl]amino}phenyl)sulfonyl]-4-(4-oxo-1,2,3-benzotriazin-3(4H)-yl)butanoic acid (Compound no. 100),
2-({4-[(Cyclopropylcarbonyl)amino]phenyl}sulfonyl)-4-(4-oxo-1,2,3-benzotriazin-3(4H)-yl)butanoic acid (Compound no. 101),
4-(4-Oxo-1,2,3-benzotriazin-3(4H)-yl)-2-({4-[(thiophen-2-ylcarbonyl)amino]phenyl}sulfonyl)butanoic acid (Compound no. 102),
2-({4-[(Cyclopentylcarbonyl)amino]phenyl}sulfonyl)-4-(4-oxo-1,2,3-benzotriazin-3(4H)-yl)butanoic acid (Compound no. 103),
2-{[4-({[4-Fluoro-3-(trifluoromethyl)phenyl]carbonyl}amino)phenyl]sulfonyl}-4-(4-oxo-1,2,3-benzotriazin-3(4H)-yl)butanoic acid (Compound no. 104),
2-[(3′-Fluoro-4′-methoxybiphenyl-4-yl)sulfanyl]-4-(7-methyl-4-oxo-1,2,3-benzotriazin-3(4H)-yl)butanoic acid (Compound no. 105),
2-[(4′-Chlorobiphenyl-4-yl)sulfanyl]-4-(6-methoxy-4-oxo-1,2,3-benzotriazin-3(4H)-yl)butanoic acid (Compound no. 106),
2-[(4′-Chlorobiphenyl-4-yl)sulfanyl]-4-(6-methyl-4-oxo-1,2,3-benzotriazin-3(4H)-yl)butanoic acid (Compound no. 107),
2-[(3′-Fluoro-4′-methoxybiphenyl-4-yl)sulfanyl]-4-(6-methyl-4-oxo-1,2,3-benzotriazin-3(4H)-yl)butanoic acid (Compound no. 108),
2-[(3′-Fluoro-4′-methoxybiphenyl-4-yl)sulfanyl]-4-(4-oxo-1,2,3-benzotriazin-3(4H)-yl)butanoic acid (Compound no. 109),
4-(6-Methyl-4-oxo-1,2,3-benzotriazin-3(4H)-yl)-2-{[4′-(trifluoromethyl)biphenyl-4-yl]sulfanyl}butanoic acid (Compound no. 110),
2-[(3′,4′-Dichlorobiphenyl-4-yl)sulfanyl]-4-(6-methyl-4-oxo-1,2,3-benzotriazin-3(4H)-yl)butanoic acid (Compound no. 111),
2-[(4′-Methoxy-3′-methylbiphenyl-4-yl)sulfanyl]-4-(6-methyl-4-oxo-1,2,3-benzotriazin-3(4H)-yl)butanoic acid (Compound no. 112),
2-[(3′,4′-Difluorobiphenyl-4-yl)sulfanyl]-4-(6-methyl-4-oxo-1,2,3-benzotriazin-3(4H)-yl)butanoic acid (Compound no. 113),
2-[(3′-Fluoro-4′-methylbiphenyl-4-yl)sulfanyl]-4-(6-methyl-4-oxo-1,2,3-benzotriazin-3(4H)-yl)butanoic acid (Compound no. 114),
2-[(4′-Fluorobiphenyl-4-yl)sulfanyl]-4-(6-methyl-4-oxo-1,2,3-benzotriazin-3(4H)-yl)butanoic acid (Compound no. 115),
2-[(4′-Ethylbiphenyl-4-yl)sulfanyl]-4-(6-methyl-4-oxo-1,2,3-benzotriazin-3(4H)-yl)butanoic acid (Compound no. 116),
2-[(4′-Methoxybiphenyl-4-yl)sulfanyl]-4-(6-methyl-4-oxo-1,2,3-benzotriazin-3(4H)-yl)butanoic acid (Compound no. 117),
2-[(4′-Fluoro-3′-methylbiphenyl-4-yl)sulfanyl]-4-(6-methyl-4-oxo-1,2,3-benzotriazin-3(4H)-yl)butanoic acid (Compound no. 118),
4-(4-Oxo-1,2,3-benzotriazin-3(4H)-yl)-2-{[4′-(trifluoromethyl)biphenyl-4-yl]sulfanyl}butanoic acid (Compound no. 119),
4-(8-Methyl-4-oxo-1,2,3-benzotriazin-3(4H)-yl)-2-{[4′-(trifluoromethoxy)biphenyl-4-yl]sulfanyl}butanoic acid (Compound no. 120),
4-(7-Methyl-4-oxo-1,2,3-benzotriazin-3(4H)-yl)-2-{[4′-(trifluoromethoxy)biphenyl-4-yl]sulfanyl}butanoic acid (Compound no. 121),
4-(6-Methoxy-4-oxo-1,2,3-benzotriazin-3(4H)-yl)-2-{[4′-(trifluoromethoxy)biphenyl-4-yl]sulfanyl}butanoic acid (Compound no. 122),
4-(7-Methoxy-4-oxo-1,2,3-benzotriazin-3(4H)-yl)-2-{[4′-(trifluoromethoxy)biphenyl-4-yl]sulfanyl}butanoic acid (Compound no. 123),
2-[(3′,4′-Difluorobiphenyl-4-yl)sulfanyl]-4-(6-methoxy-4-oxo-1,2,3-benzotriazin-3(4H)-yl)butanoic acid (Compound no. 124),
2-[(3′-Fluoro-4′-methylbiphenyl-4-yl)sulfanyl]-4-(6-methoxy-4-oxo-1,2,3-benzotriazin-3(4H)-yl)butanoic acid (Compound no. 125),
2-[(4′-Fluoro-3′-methylbiphenyl-4-yl)sulfanyl]-4-(6-methoxy-4-oxo-1,2,3-benzotriazin-3(4H)-yl)butanoic acid (Compound no. 126),
2-[(3′,4′-Dichlorobiphenyl-4-yl)sulfanyl]-4-(6-methoxy-4-oxo-1,2,3-benzotriazin-3(4H)-yl)butanoic acid (Compound no. 127),
2-[(3′-Fluoro-4′-methoxybiphenyl-4-yl)sulfanyl]-4-(6-methoxy-4-oxo-1,2,3-benzotriazin-3(4H)-yl)butanoic acid (Compound no. 128),
2-{[4′-chloro-3′-(trifluoromethyl)biphenyl-4-yl]sulfanyl}-4-(6-methoxy-4-oxo-1,2,3-benzotriazin-3(4H)-yl)butanoic acid (Compound no. 129),
2-[(4′-Ethylbiphenyl-4-yl)sulfanyl]-4-(6-methoxy-4-oxo-1,2,3-benzotriazin-3(4H)-yl)butanoic acid (Compound no. 130),
2-[(3′,4′-Dimethylbiphenyl-4-yl)sulfanyl]-4-(6-methoxy-4-oxo-1,2,3-benzotriazin-3(4H)-yl)butanoic acid (Compound no. 131),
4-(6-Methoxy-4-oxo-1,2,3-benzotriazin-3(4H)-yl)-2-[(4′-methylbiphenyl-4-yl)sulfanyl]butanoic acid (Compound no. 132),
4-(6-Methoxy-4-oxo-1,2,3-benzotriazin-3(4H)-yl)-2-{[4′-(trifluoromethyl)biphenyl-4-yl]sulfanyl}butanoic acid (Compound no. 133),
2-[(4′-Ethylbiphenyl-4-yl)sulfanyl]-4-(8-methyl-4-oxo-1,2,3-benzotriazin-3(4H)-yl)butanoic acid (Compound no. 134),
2-[(3′-Fluoro-4′-methylbiphenyl-4-yl)sulfanyl]-4-(8-methyl-4-oxo-1,2,3-benzotriazin-3(4H)-yl)butanoic acid (Compound no. 135),
2-{[4-(6-Methoxypyridin-3-yl)phenyl]sulfanyl}-4-(8-methyl-4-oxo-1,2,3-benzotriazin-3(4H)-yl)butanoic acid (Compound no. 136),
2-[(4′-Methylbiphenyl-4-yl)sulfanyl]-4-(8-methyl-4-oxo-1,2,3-benzotriazin-3(4H)-yl)butanoic acid (Compound no. 137),
2-[(4′-Chlorobiphenyl-4-yl)sulfanyl]-4-(8-methyl-4-oxo-1,2,3-benzotriazin-3(4H)-yl)butanoic acid (Compound no. 138),
2-[(4′-Fluorobiphenyl-4-yl)sulfanyl]-4-(8-methyl-4-oxo-1,2,3-benzotriazin-3(4H)-yl)butanoic acid (Compound no. 139),
2-[(4′-Fluoro-3′-methylbiphenyl-4-yl)sulfanyl]-4-(8-methyl-4-oxo-1,2,3-benzotriazin-3(4H)-yl)butanoic acid (Compound no. 140),
2-[(3′,4′-Difluorobiphenyl-4-yl)sulfanyl]-4-(8-methyl-4-oxo-1,2,3-benzotriazin-3(4H)-yl)butanoic acid (Compound no. 141),
2-[(3′,4′-Dimethoxybiphenyl-4-yl)sulfanyl]-4-(8-methyl-4-oxo-1,2,3-benzotriazin-3(4H)-yl)butanoic acid (Compound no. 142),
2-[(3′,4′-Dimethylbiphenyl-4-yl)sulfanyl]-4-(8-methyl-4-oxo-1,2,3-benzotriazin-3(4H)-yl)butanoic acid (Compound no. 143),
4-(8-Methyl-4-oxo-1,2,3-benzotriazin-3(4H)-yl)-2-{[4′-(trifluoromethyl)biphenyl-4-yl]sulfanyl}butanoic acid (Compound no. 144),
2-[(3′-Fluoro-4′-methoxybiphenyl-4-yl)sulfanyl]-4-(8-methyl-4-oxo-1,2,3-benzotriazin-3(4H)-yl)butanoic acid (Compound no. 145),
4-(5-Chloro-4-oxo-1,2,33-benzotriazin-3(4H)-yl)-2-[(3′-fluoro-4′-methoxybiphenyl-4-yl)sulfanyl]butanoic acid (Compound no. 146),
4-(7-Chloro-4-oxo-1,2,3-benzotriazin-3(4H)-yl)-2-[(3′-fluoro-4′-methoxybiphenyl-4-yl)sulfanyl]butanoic acid (Compound no. 147),
2-{[4-(6-Methoxypyridin-3-yl)phenyl]sulfanyl}-4-(4-oxo-1,2,3-benzotriazin-3(4H)-yl)butanoic acid (Compound no. 148),
2-[(4′-Methoxybiphenyl-4-yl)sulfanyl]-4-(4-oxo-1,2,3-benzotriazin-3(4H)-yl)butanoic acid (Compound no. 149),
2-[(3′-Fluoro-4′-methylbiphenyl-4-yl)sulfanyl]-4-(4-oxo-1,2,3-benzotriazin-3(4H)-yl)butanoic acid (Compound no. 150),
2-[(3′,4′-Difluorobiphenyl-4-yl)sulfanyl]-4-(4-oxo-1,2,3-benzotriazin-3(4H)-yl)butanoic acid (Compound no. 151),
2-[(3′-Methoxybiphenyl-4-yl)sulfanyl]-4-(4-oxo-1,2,3-benzotriazin-3(4H)-yl)butanoic acid (Compound no. 152),
2-[(4′-Fluorobiphenyl-4-yl)sulfanyl]-4-(4-oxo-1,2,3-benzotriazin-3(4H)-yl)butanoic acid (Compound no. 153),
2-(Biphenyl-4-ylsulfanyl)-4-(4-oxo-1,2,3-benzotriazin-3(4H)-yl)butanoic acid (Compound no. 154),
2-[(2′,3′-Difluorobiphenyl-4-yl)sulfanyl]-4-(4-oxo-1,2,3-benzotriazin-3(4H)-yl)butanoic acid (Compound no. 155),
2-{[(4′-Chlorobiphenyl-4-yl)methyl]sulfanyl}-4-(4-oxo-1,2,3-benzotriazin-3(4H)-yl)butanoic acid (Compound no. 156),
2-{[(4′-Chlorobiphenyl-4-yl)methyl]sulfanyl}-4-(7-methyl-4-oxo-1,2,3-benzotriazin-3(4H)-yl)butanoic acid (Compound no. 157),
2-{[(4′-Chlorobiphenyl-4-yl)methyl]sulfanyl}-4-(7-methoxy-4-oxo-1,2,3-benzotriazin-3(4H)-yl)butanoic acid (Compound no. 158),
2-{[(4′-Chlorobiphenyl-4-yl)methyl]sulfanyl}-4-(6-fluoro-4-oxo-1,2,3-benzotriazin-3(4H)-yl)butanoic acid (Compound no. 159),
2-{[(4′-Chlorobiphenyl-4-yl)methyl]sulfanyl}-4-(8-methyl-4-oxo-1,2,3-benzotriazin-3(4H)-yl)butanoic acid (Compound no. 160),
2-{[(4′-Chlorobiphenyl-4-yl)methyl]sulfanyl}-4-[4-oxo-7-(trifluoromethyl)-1,2,3-benzotriazin-3(4H)-yl]butanoic acid (Compound no. 161),
2-{[(4′-Chlorobiphenyl-4-yl)methyl]sulfanyl}-4-(5-chloro-4-oxo-1,2,3-benzotriazin-3(4H)-yl)butanoic acid (Compound no. 162),
2-{[2-(4′-Chlorobiphenyl-4-yl)ethyl]sulfanyl}-4-(7-chloro-4-oxo-1,2,3-benzotriazin-3(4H)-yl)butanoic acid (Compound no. 163),
2-{[2-(4′-Chlorobiphenyl-4-yl)ethyl]sulfanyl}-4-(7-methyl-4-oxo-1,2,3-benzotriazin-3(4H)-yl)butanoic acid (Compound no. 164),
2-{[2-(4′-Chlorobiphenyl-4-yl)ethyl]sulfanyl}-4-(8-methyl-4-oxo-1,2,3-benzotriazin-3(4H)-yl)butanoic acid (Compound no. 165),
2-[(4′-tert-Butylbiphenyl-4-yl)sulfanyl]-4-(4-oxo-1,2,3-benzotriazin-3(4H)-yl)butanoic acid (Compound no. 166),
2-[(4′-Ethylbiphenyl-4-yl)sulfanyl]-4-(4-oxo-1,2,3-benzotriazin-3(4H)-yl)butanoic acid (Compound no. 167),
4-(4-Oxo-1,2,3-benzotriazin-3(4H)-yl)-2-{[4′-(propan-2-yl)biphenyl-4-yl]sulfanyl}butanoic acid (Compound no. 168),
4-(4-Oxo-1,2,3-benzotriazin-3(4H)-yl)-2-{[4′-(trifluoromethyl)biphenyl-4-yl]sulfanyl}butanoic acid (Compound no. 169),
4-(4-Oxo-1,2,3-benzotriazin-3(4H)-yl)-2-{[4′-(trifluoromethoxy)biphenyl-4-yl]sulfanyl}butanoic acid (Compound no. 170),
2-[(3′-Fluoro-4′-methoxybiphenyl-4-yl)sulfanyl]-4-(7-methoxy-4-oxo-1,2,3-benzotriazin-3(4H)-yl)butanoic acid (Compound no. 171),
4-(7-Methoxy-4-oxo-1,2,3-benzotriazin-3(4H)-yl)-2-{[4-(6-methoxypyridin-3-yl)phenyl]sulfanyl}butanoic acid (Compound no. 172),
4-(7-Methoxy-4-oxo-1,2,3-benzotriazin-3(4H)-yl)-2-{[4′-(trifluoromethyl)biphenyl-4-yl]sulfanyl}butanoic acid (Compound no. 173),
2-[(4′-Methoxybiphenyl-4-yl)sulfanyl]-4-(7-methoxy-4-oxo-1,2,3-benzotriazin-3(4H)-yl)butanoic acid (Compound no. 174),
2-[(3′-Fluoro-4′-methylbiphenyl-4-yl)sulfanyl]-4-(7-methoxy-4-oxo-1,2,3-benzotriazin-3(4H)-yl)butanoic acid (Compound no. 175),
4-(7-Methoxy-4-oxo-1,2,3-benzotriazin-3(4H)-yl)-2-{[4-(1-methyl-1H-pyrazol-4-yl)phenyl]sulfanyl}butanoic acid (Compound no. 176),
2-[(3′,4′-Dimethoxybiphenyl-4-yl)sulfanyl]-4-(7-methoxy-4-oxo-1,2,3-benzotriazin-3(4H)-yl)butanoic acid (Compound no. 177),
2-[(4′-Ethylbiphenyl-4-yl)sulfanyl]-4-(7-methoxy-4-oxo-1,2,3-benzotriazin-3(4H)-yl)butanoic acid (Compound no. 178),
2-[(4′-Fluoro-3′-methylbiphenyl-4-yl)sulfanyl]-4-(7-methoxy-4-oxo-1,2,3-benzotriazin-3(4H)-yl)butanoic acid (Compound no. 179),
2-[(4′-Chlorobiphenyl-4-yl)sulfanyl]-4-(7-methoxy-4-oxo-1,2,3-benzotriazin-3(4H)-yl)butanoic acid (Compound no. 180),
2-[(3′,4′-Dichlorobiphenyl-4-yl)sulfanyl]-4-(7-methoxy-4-oxo-1,2,3-benzotriazin-3(4H)-yl)butanoic acid (Compound no. 181),
2-[(4-{[(4-Chlorophenyl)carbonyl]amino}phenyl)sulfanyl]-4-(4-oxo-1,2,3-benzotriazin-3(4H)-yl)butanoic acid (Compound no. 182),
2-[(4-{[(3-Methoxyphenyl)acetyl]amino}phenyl)sulfonyl]-4-(4-oxo-1,2,3-benzotriazin-3(4H)-yl)butanoic acid (Compound no. 183),
2-[(4-{[(2,5-Dimethoxyphenyl)acetyl]amino}phenyl)sulfonyl]-4-(4-oxo-1,2,3-benzotriazin-3(4H)-yl)butanoic acid (Compound no. 184),
4-(4-Oxo-1,2,3-benzotriazin-3(4H)-yl)-2-({4-(phenylacetyl)amino]phenyl}sulfonyl)butanoic acid (Compound no. 185),
2-(4-Fluorobenzyl)-2-[(2-{4-[(4-fluorobenzyl)oxy]phenyl}ethyl)sulfonyl]-4-(4-oxo-1,2,3-benzotriazin-3(4H)-yl)butanoic acid (Compound no. 186),
4-(6-Methoxy-4-oxo-1,2,3-benzotriazin-3(4H)-yl)-2-[(4-{[(4-methylphenyl)carbonyl]amino}phenyl)sulfanyl]butanoic acid (Compound no. 187),
4-(8-Methyl-4-oxo-1,2,3-benzotriazin-3(4H)-yl)-2-[(4-{[(4-methylphenyl)carbonyl]amino}phenyl)sulfanyl]butanoic acid (Compound no. 188),
4-(6-Methoxy-4-oxo-1,2,3-benzotriazin-3(4H)-yl)-2-[(4-{[(4-methylphenyl)carbonyl]amino}phenyl)sulfonyl]butanoic acid (Compound no. 189),
4-(8-Methyl-4-oxo-1,2,3-benzotriazin-3(4H)-yl)-2-[(4-{[(4-methylphenyl)carbonyl]amino}phenyl)sulfonyl]butanoic acid (Compound no. 190),
2-(3-Fluorobenzyl)-2-[(2-{4-[(3-fluorobenzyl)oxy]phenyl}ethyl)sulfonyl]-4-(4-oxo-1,2,3-benzotriazin-3(4H)-yl)butanoic acid (Compound no. 191),
4-(6-Methyl-4-oxo-1,2,3-benzotriazin-3(4H)-yl)-2-[(4-{[(4-methylphenyl)carbonyl]amino}phenyl)sulfanyl]butanoic acid (Compound no. 192),
4-(7-Methyl-4-oxo-1,2,3-benzotriazin-3(4H)-yl)-2-[(4-{[(4-methylphenyl)carbonyl]amino}phenyl)sulfanyl]butanoic acid (Compound no. 193),
4-(6-Fluoro-4-oxo-1,2,3-benzotriazin-3(4H)-yl)-2-[(4-{[(4-methylphenyl)carbonyl]amino}phenyl)sulfanyl]butanoic acid (Compound no. 194),
2-{[2-(4′-Chlorobiphenyl-4-yl)ethyl]sulfanyl}-4-(6-fluoro-4-oxo-1,2,3-benzotriazin-3(4H)-yl)butanoic acid (Compound no. 195),
2-{[2-(4′-Chlorobiphenyl-4-yl)ethyl]sulfanyl}-4-(6-methyl-4-oxo-1,2,3-benzotriazin-3(4H)-yl)butanoic acid (Compound no. 196),
2-{[2-(4′-Chlorobiphenyl-4-yl)ethyl]sulfonyl}-4-(7-methyl-4-oxo-1,2,3-benzotriazin-3(4H)-yl)butanoic acid (Compound no. 197),
2-(2-Chlorobenzyl)-2-[(2-{4-[(2-chlorobenzyl)oxy]phenyl}ethyl)sulfonyl]-4-(4-oxo-1,2,3-benzotriazin-3(4H)-yl)butanoic acid (Compound no. 198),
2-{[2-(4′-Chlorobiphenyl-4-yl)ethyl]sulfonyl}-4-(6-methyl-4-oxo-1,2,3-benzotriazin-3(4H)-yl)butanoic acid (Compound no. 199),
4-(6-Methyl-4-oxo-1,2,3-benzotriazin-3(4H)-yl)-2-[(4-{[(4-methylphenyl)carbonyl]amino}phenyl)sulfonyl]butanoic acid (Compound no. 200),
4-(7-Methyl-4-oxo-1,2,3-benzotriazin-3(4H)-yl)-2-[(4-{[(4-methylphenyl)carbonyl]amino}phenyl)sulfonyl]butanoic acid (Compound no. 201),
2-[(4-{[(4-Methoxyphenyl)carbonyl]amino}phenyl)sulfanyl]-4-(6-methyl-4-oxo-1,2,3-benzotriazin-3(4H)-yl)butanoic acid (Compound no. 202),
2-[(4-{[(4-Fluorophenyl)carbonyl]amino}phenyl)sulfanyl]-4-(6-methyl-4-oxo-1,2,3-benzotriazin-3(4H)-yl)butanoic acid (Compound no. 203),
2-[(4-{[(3,4-Dichlorophenyl)carbonyl]amino}phenyl)sulfanyl]-4-(6-methyl-4-oxo-1,2,3-benzotriazin-3(4H)-yl)butanoic acid (Compound no. 204),
2-[(4-{[(4-Ethylphenyl)carbonyl]amino}phenyl)sulfanyl]-4-(6-methyl-4-oxo-1,2,3-benzotriazin-3(4H)-yl)butanoic acid (Compound no. 205),
2-[(4-{[(4-Methoxyphenyl)carbonyl]amino}phenyl)sulfonyl]-4-(6-methyl-4-oxo-1,2,3-benzotriazin-3(4H)-yl)butanoic acid (Compound no. 206),
2-[(4-{[(3,4-Dichlorophenyl)carbonyl]amino}phenyl)sulfonyl]-4-(6-methyl-4-oxo-1,2,3-benzotriazin-3(4H)-yl)butanoic acid (Compound no. 207),
2-[(4-{[(4-Ethylphenyl)carbonyl]amino}phenyl)sulfonyl]-4-(6-methyl-4-oxo-1,2,3-benzotriazin-3(4H)-yl)butanoic acid (Compound no. 208),
2-[(4-{[(4-Fluorophenyl)carbonyl]amino}phenyl)sulfonyl]-4-(6-methyl-4-oxo-1,2,3-benzotriazin-3(4H)-yl)butanoic acid (Compound no. 209),
2-[(4-{[(4-Chlorophenyl)carbonyl]amino}phenyl)sulfonyl]-4-(6-methyl-4-oxo-1,2,3-benzotriazin-3(4H)-yl)butanoic acid (Compound no. 210),
2-[(4′-Chlorobiphenyl-4-yl)oxy]-4-(4-oxo-1,2,3-benzotriazin-3(4H)-yl)butanoic acid (Compound no. 211),
2-[(4′-Chlorobiphenyl-4-yl)methoxy]-4-(6-fluoro-4-oxo-1,2,3-benzotriazin-3(4H)-yl)butanoic acid (Compound no. 212),
2-[(4′-Chlorobiphenyl-4-yl)oxy]-4-(7-chloro-4-oxo-1,2,3-benzotriazin-3(4H)-yl)butanoic acid (Compound no. 213),
2-[(4′-Chlorobiphenyl-4-yl)oxy]-4-[4-oxo-7-(trifluoromethyl)-1,2,3-benzotriazin-3(4H)-yl]butanoic acid (Compound no. 214),
2-[(4′-Chlorobiphenyl-4-yl)oxy]-4-(5-fluoro-4-oxo-1,2,3-benzotriazin-3(4H)-yl)butanoic acid (Compound no. 215),
2-[(4′-Chlorobiphenyl-4-yl)oxy]-4-(5-chloro-4-oxo-1,2,3-benzotriazin-3(4H)-yl)butanoic acid (Compound no. 216),
2-[(4′-Chlorobiphenyl-4-yl)oxy]-4-(6-methoxy-4-oxo-1,2,3-benzotriazin-3(4H)-yl)butanoic acid (Compound no. 217),
2-[(4′-Chlorobiphenyl-4-yl)oxy]-4-(5-methyl-4-oxo-1,2,3-benzotriazin-3(4H)-yl)butanoic acid (Compound no. 218),
(2R)-2-[(4′-Chlorobiphenyl-4-yl)oxy]-4-(6-fluoro-4-oxo-1,2,3-benzotriazin-3(4H)-yl)butanoic acid (Compound no. 219),
(2R)-2-[(4′-Chlorobiphenyl-4-yl)oxy]-4-(7-chloro-4-oxo-1,2,3-benzotriazin-3(4H)-yl)butanoic acid (Compound no. 220),
(2R)-2-[(4′-Chlorobiphenyl-4-yl)oxy]-4-(6-methoxy-4-oxo-1,2,3-benzotriazin-3(4H)-yl)butanoic acid (Compound no. 221),
(2R)-2-[(4′-Chlorobiphenyl-4-yl)oxy]-4-(6,7-difluoro-4-oxo-1,2,3-benzotriazin-3(4H)-yl)butanoic acid (Compound no. 222),
2-[(4′-Chlorobiphenyl-4-yl)methoxy]-4-(7-chloro-4-oxo-1,2,3-benzotriazin-3(4H)-yl)butanoic acid (Compound no. 223),
2-[(4′-Chlorobiphenyl-4-yl)methoxy]-4-(7-methoxy-4-oxo-1,2,3-benzotriazin-3(4H)-yl)butanoic acid (Compound no. 224),
(2R)-2-[(4′-Chlorobiphenyl-4-yl)oxy]-4-(2,4-dioxo-2H-1,3-benzoxazin-3(4H)-yl)butanoic acid (Compound no. 225),
(2R)-2-[(4′-Chlorobiphenyl-4-yl)oxy]-4-(1-oxophthalazin-2(1H)-yl)butanoic acid (Compound no. 226),
2-[(4′-Chlorobiphenyl-4-yl)methoxy]-4-(4-oxo-1,2,3-benzotriazin-3(4H)-yl)butanoic acid (Compound no. 227),
2-[(4′-Chlorobiphenyl-4-yl)methoxy]-4-(1-oxophthalazin-2(1H)-yl)butanoic acid (Compound no. 228),
2-[(4′-Chlorobiphenyl-4-yl)methoxy]-4-(2,4-dioxo-2H-1,3-benzoxazin-3(4H)-yl)butanoic acid (Compound no. 229),
   including racemates, enantiomers and diastereomers thereof, or a pharmaceutically acceptable salt of any one thereof.

In another aspect, provided herein are pharmaceutical composition comprising therapeutically effective amounts of one or more compounds described herein together with one or more pharmaceutically acceptable carriers, excipients or diluents.

In another aspect, provided herein are compounds according to Formula I/Ia for use in medicine.

In another aspect, provided herein are compounds according to Formula I/Ia for use in treating or preventing various inflammatory and allergic diseases comprising administering to a mammal in need thereof.

In another aspect, provided herein are compounds according to Formula I/Ia wherein various inflammatory and allergic diseases are asthma, rheumatoid arthritis, COPD, dry eye, rhinitis, osteoarthritis, psoriatic arthritis, psoriasis, pulmonary fibrosis, pulmonary inflammation, acute respiratory distress syndrome, periodontitis, multiple sclerosis, gingivitis, atherosclerosis, dry eye, neointimal proliferation associated with restenosis and ischemic heart failure, stroke, renal disease or tumor metastasis.

In yet another aspect, the present invention relates to the therapeutically effective dose of a compound of Formula I/Ia in combination with one or more of other therapeutic agents used for treating various inflammatory and allergic diseases. Examples of such therapeutic agents include, but are not limited to:
1) Anti-inflammatory agents, experimental or commercial (i) such as nonsteroidal anti-inflammatory agents piroxicam, diclofenac, propionic acids, fenamates, pyrazolones, salicylates, PDE-4/p38 MAP Kinase/Cathepsin inhibitors, CCR-3 antagonists, iNOS inhibitors, tryptase and elastase inhibitors, beta-2 integrin antagonists, cell adhesion inhibitors (especially ICAM), adenosine 2a agonists; (ii) leukotrienes LTC4/LTD4/LTE4/LTB4-Inhibitors, 5-lipoxygenase inhibitors and PAF-receptor antagonists; (iii) Cox-2 inhibitors; (iv) other MMP inhibitors; (v) interleukin-I inhibitors; (vi) corticosteroids such as alclometasone, amcinonide, amelometasone, beclometasone, betamethasone, budesonide, ciclesonide, clobetasol, cloticasone, cyclomethasone, deflazacort, deprodone, dexbudesonide, diflorasone, difluprednate, fluticasone, flunisolide, halometasone, halopredone, hydrocortisone, methylprednisolone, mometasone, prednicarbate, prednisolone, rimexolone, tixocortol, triamcinolone, ulobetasol, rofleponide, GW 215864, KSR 592, ST-126, dexamethasone and pharmaceutically acceptable salts, solvates thereof. Preferred corticosteroids include, for example, flunisolide, beclomethasone, triamcinolone, budesonide, fluticasone, mometasone, ciclesonide, and dexamethasone; (vii) Cathepsin-S inhibitors;
2) Beta-agonists, experimental or commercial (i) suitable β2-agonists include, for example, one or more of albuterol, salbutamol, biltolterol, pirbuterol levosalbutamol, tulobuterol, terbutaline, bambuterol, metaproterenol, fenoterol, salmeterol, carmoterol, arformoterol, formoterol, and their pharmaceutically acceptable salts or solvates thereof. One or more β2-agonists may be chosen from those in the art or subsequently discovered. (ii) the β2-agonists may include one or more compounds described in, for example, U.S. Pat. Nos. 3,705,233; 3,644,353; 3,642,896; 3,700,681; 4,579,985; 3,994,974; 3,937,838; 4,419,364; 5,126,375; 5,243,076; 4,992,474; and 4,011,258;
3) antihypertensive agents, (i) ACE inhibitors, e.g., enalapril, lisinopril, valsartan, telmisartan and quinapril; (ii) angiotensin II receptor antagonists and agonists, e.g., losartan, candesartan, irbesartan, valsartan, and eprosartan; (iii) β-blockers; and (iv) calcium channel blockers;
4) immunosuppressive agents, for example, cyclosporine, azathioprine and methotrexate, anti-inflammatory corticosteroids; and
5) anti-infective agents (e.g. antibiotics, antivirals).

The following definitions apply to terms as used herein:

The term “alkyl” refers to a straight or branched fully saturated hydrocarbon chain which is optionally substituted by one or more halo atoms, and which has 1 to 20 carbon atoms unless otherwise specified. This term is exemplified by groups, such as methyl, ethyl, n-propyl, iso-propyl, n-butyl, iso-butyl, t-butyl, n-hexyl, n-decyl, n-tetradecyl, trifluoromethyl, chloroethyl, and the like.

The term “alkenyl”, unless otherwise specified, refers to a branched or unbranched unsaturated hydrocarbon group containing at least one double bond with cis or trans geometry and preferably having 2 to 20 carbon atoms. Examples of alkenyl group include ethenyl, 2-propenyl and isopropenyl.

The term “cycloalkyl” refers to a non-aromatic cyclic group having 3 to 20 ring carbon atoms and forms one to three rings and may optionally contain one or more olefinic bonds. Polycyclic ring systems may be a spiro, fused or bridged arrangement. Cycloalkyl groups include, by way of example, cyclopropyl, cyclobutyl, cyclopentyl, cyclohexyl, cycloheptyl, cyclooctyl, cyclopentenyl, cyclohexenyl, adamantlyl, bicyclo[2.2.1]heptanyl, bicyclo[2.2.2]octane, tricycle[3.3.1.1]decane, and the like.

The term “aryl” refers to an aromatic system having from 6 to 14 carbon atoms and up to three rings which may be fused or directly joined. Representative examples of such aryl group include, but are not limited to, phenyl, biphenyl, naphthyl, phenanthrene, anthracenyl, azulenyl, and indanyl. Aryl group may also comprise one or more rings which are not fully aromatic and examples of such system are indane, indene, 2,3 dihydrobenzofuran and 1,2,3,4-tetrahydronaphthalene.

The term “heteroaryl” refers to an aromatic system having from 5 to 14 membered carbon atoms and up to three rings, which may be fused or directly joined, and containing from one to eight heteroatoms selected from N, O and S. Examples of heteroaryl groups are yridinyl, quinolinyl, oxazolyl, imidazolyl, pyrrolyl, thiophenyl, 1,2,3-triazolyl, 1,2,4-triazolyl, tetrazolyl, thiazolyl, oxadiazolyl, benzoimidazolyl, thiadiazolyl, pyridazinyl, pyrazinyl, thienyl, soxazolyl, triazinyl, furanyl, benzofuranyl, indolyl, benzothiazolyl, benzoxazolyl, and the like.

The term “heterocyclyl” refers to a non-aromatic monocyclic or polycyclic ring system, which may be fused, spiro or bridged having 3 to 12 ring atoms and up to eight heteroatoms selected from N, O and S. Examples of heterocyclyl ring systems include piperidine, morpholine, piperazine, isoquinoline, oxazolidine, tetrahydrofuran, dihydrofuran, dihydropyridine, dihydroisoxazole, dihydrobenzofuran, azabicyclohexane, dihydroindole, tetrahydroquinoline, pyrrolidine, azepine, azetidine, aziridine, tetrahydropyridine, benzthiazine, benzoxazinyl, isoindoline, azabicycle[3.1.0]hexyl, phenoxazine, tetrahydropyran, 1,4-dioxane, and the like.

The terms “cycloalkylalkyl”, “arylalkyl”, “heteroarylalkyl”, “heterocyclylalkyl” refer respectively to cycloalkyl, aryl, heteroaryl or heterocyclyl group linked the remainder of the molecule via an alkyl group.

The term “amino” refers to —NH₂.

The term “alkoxy” denotes the group O-alkyl, wherein alkyl is the same as defined above.

The term “halogen” or “halo” refers to fluorine, chlorine, bromine or iodine.

The term “halogeno-C₁-C₆ alkyl” refers to C₁-C₆ alkyl of which one or more hydrogen(s) is/are replaced by halogen.

The term “halogeno C₁-C₆ alkoxy” refers to as halogen atom bonded to C₁-C₆ alkoxy group. Examples of such groups include trifluoromethoxy, trichloromethoxy, difluoromethoxy, fluoromethoxy, 2,2,2-trifluoroethoxy, 2-bromoethoxy, etc.

The term “hydroxyl” or “hydroxy” refers to —OH.

The term “thiol” refers to the group —SH.

The term “alkylthiol” refers to a thiol group when hydrogen is replaced by alkyl, for example, methylthio, ethylthio, propylthio, t-butylthio, cyclopropylthio, and the like.

The term “cyano” refers to C≡N.

The term “azido” refers to N═N═N

The term “leaving group” refers to groups that exhibit or potentially exhibit the properties of being labile under the synthetic conditions and also of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals, and the like.

The term “protecting groups” refers to moieties that prevent chemical reaction at a S25 location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y. The species of the carboxylic protecting groups, amino protecting groups or hydroxy protecting groups employed are not critical, as long as the derivatised moiety/moieties is/are stable to conditions of subsequent reactions and can be removed without disrupting the remainder of the molecule.

Compounds described herein can contain one or more asymmetric carbon atoms and thus occur as diastereomers. These compounds can also exist as conformers/rotamers. All such isomeric forms of these compounds are included herein. Each stereogenic carbon may be of the R or S configuration. Although the specific compounds exemplified in this application may be depicted in a particular stereochemical configuration, compounds having either the opposite stereochemistry at any given chiral center or mixtures thereof are envisioned.

The term “pharmaceutically acceptable salts” forming part of this invention includes the salts of carboxylic acid moiety, which may be prepared by reacting the compound with an appropriate base to provide corresponding base addition salts. Examples of such bases are alkali metal hydroxide including potassium hydroxide, sodium hydroxide and lithium hydroxide; alkaline earth metal hydroxides, such as magnesium hydroxide and calcium hydroxide. Further, the salts of organic bases, such as lysine, arginine, guanidine, ethanolamine, choline, and the like; inorganic bases, e.g., ammonium or substituted ammonium salts are also included. Wherever appropriate, compounds of the present invention may also form the acid addition salts by treating the said compounds with pharmaceutically acceptable organic and inorganic acids, e.g., hydrohalides, such as hydrochloride, hydrobromide, hydroiodide; other mineral acids and their corresponding salts, such as sulphate, nitrate, phosphate, etc.; and alkyl and mono-arylsulphonates, such as ethane sulphonate, toluene sulphonate and benzene sulphonate; and other organic acids and their corresponding salts, such as acetate, tartarate, maleate, succinate, citrate, etc. The salt forms differ from the compound described herein in certain physical properties, such as solubility, but the salts are otherwise equivalent for the purpose of this invention.

The term “pharmaceutically acceptable solvates” refers to solvates with water (i.e., hydrates) or pharmaceutically acceptable solvents, for example, solvates with ethanol, and the like. Such solvates are also encompassed within the scope of the disclosure. Furthermore, some of the crystalline forms for compounds described herein may exist as polymorphs and as such are intended to be included in the scope of the disclosure.

The term “polymorphs” includes all crystalline forms as well as amorphous forms for compounds described herein and as such are included in the present invention.

The term “pharmaceutically acceptable carriers” is intended to include non-toxic, inert solid, semi-solid or liquid filler, diluent, encapsulating material or formulation auxiliary of any type.

The term “pharmaceutically acceptable” means approved by a regulatory agency of the federal or a state government or listed in the U.S. Pharmacopoeia or other generally recognized pharmacopoeia for use in animals, and more particularly in humans.

Examples of inflammatory conditions and autoimmune disorders in which the compounds of the invention have potentially beneficial effects in treatment methods may include, but are not limited to, diseases of the respiratory tract, such as asthma (including allergen-induced asthmatic reactions), cystic fibrosis, bronchitis (including chronic bronchitis), chronic obstructive pulmonary disease (COPD), adult respiratory distress syndrome (ARDS), chronic pulmonary inflammation, rhinitis and upper respiratory tract inflammatory disorders (URID), ventilator induced lung injury, silicosis, pulmonary sarcoidosis, idiopathic pulmonary fibrosis, bronchopulmonary dysplasia, arthritis, e.g., rheumatoid arthritis, osteoarthritis, infectious arthritis, psoriatic arthritis, traumatic arthritis, rubella arthritis, Reiter's syndrome, gouty arthritis and prosthetic joint failure, gout, acute synovitis, spondylitis and non-articular inflammatory conditions, e.g., herniated/ruptured/prolapsed intervertebral disk syndrome, bursitis, tendonitis, tenosynovitic, fibromyalgic syndrome and other inflammatory conditions associated with ligamentous sprain and regional musculoskeletal strain, inflammatory disorders of the gastrointestinal tract, e.g., ulcerative colitis, diverticulitis, Crohn's disease, inflammatory bowel diseases, irritable bowel syndrome and gastritis, multiple sclerosis, systemic lupus erythematosus, scleroderma, autoimmune exocrinopathy, autoimmune encephalomyelitis, diabetes, tumor angiogenesis and metastasis, cancer including carcinoma of the breast, colon, rectum, lung, kidney, ovary, stomach, uterus, pancreas, liver, oral, laryngeal and prostate, melanoma, acute and chronic leukemia, periodontal disease, neurodegenerative disease, Alzheimer's disease, Parkinson's disease, epilepsy, muscle degeneration, inguinal hernia, retinal degeneration, diabetic retinopathy, macular degeneration, ocular inflammation, bone resorption diseases, osteoporosis, osteopetrosis, graft vs. host reaction, allograft rejections, sepsis, endotoxemia, toxic shock syndrome, tuberculosis, usual interstitial and cryptogenic organizing pneumonia, bacterial meningitis, systemic cachexia, cachexia secondary to infection or malignancy, cachexia secondary to acquired immune deficiency syndrome (AIDS), malaria, leprosy, leishmaniasis, Lyme disease, glomerulonephritis, glomerulosclerosis, renal fibrosis, liver fibrosis, pancrealitis, hepatitis, endometriosis, pain, e.g., that associated with inflammation and/or trauma, inflammatory diseases of the skin, e.g., dermatitis, dermatosis, skin ulcers, psoriasis, eczema, systemic vasculitis, vascular dementia, thrombosis, atherosclerosis, restenosis, reperfusion injury, plaque calcification, myocarditis, aneurysm, stroke, pulmonary hypertension, left ventricular remodeling and heart failure. It will be appreciated by those skilled in the art that reference herein to treatment extends to prophylaxis as well as the treatment of established conditions.

Compounds disclosed herein may be prepared, for example, by techniques well known in the organic synthesis and familiar to a practitioner ordinarily skilled in art of this invention. In addition, the processes described herein may enable the synthesis of the compounds of the present invention. However, these may not be the only means by which the compounds described in the invention may be synthesized. Further, the various synthetic steps described herein may be performed in alternate sequences in order to furnish the desired compounds.

Accordingly, compounds of Formula X can be prepared by following Scheme I.

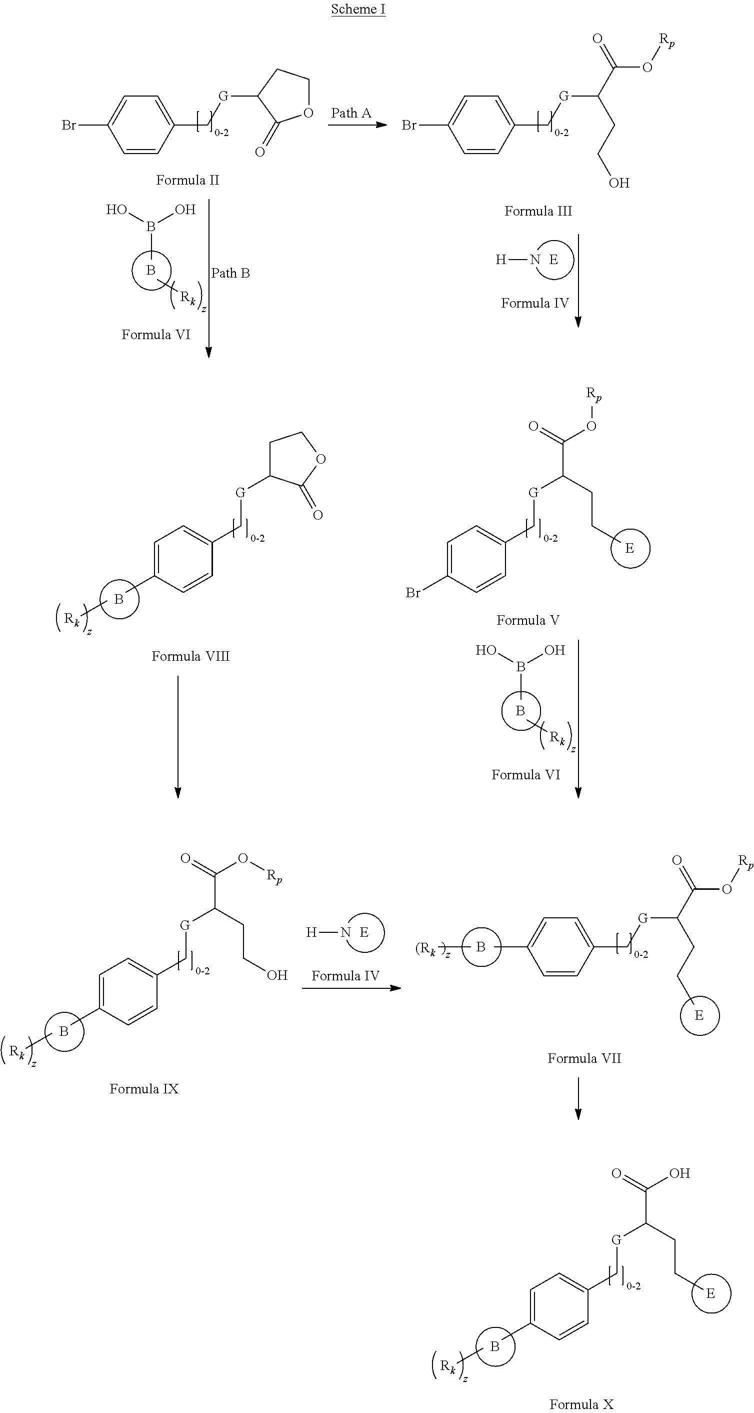

Compounds of Formula II can react through two pathways.

### Path A:

The ring opening of compound of Formula II (wherein G is O or S), gives a compound of Formula III (wherein Rₚ is carboxy protecting group such as methyl, ethyl, allyl, benzyl, t-butyl and silyl) which reacts with a compound of Formula IV (wherein

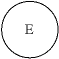

is as defined earlier where the heterocycle is N-attached) to give a compound of Formula V, which then undergoes coupling with a compound of Formula VI (wherein Rₖ is H, halo, alkyl, alkoxy, cyano, halogeno-C₁-C₆ alkyl, halogeno-C₁-C₆ alkoxy) to give a compound of Formula VII.

### Path B:

The compound of Formula II can undergo coupling with a compound of Formula VI to give a compound of Formula VIII. The ring opening of compound of Formula VIII gives a compound of Formula IX, which then reacts with a compound of Formula IV to give a compound of Formula VII.

The compound of Formula VII can then undergo hydrolysis to give a compound of Formula X.

The reaction of a compound of Formula II (Path A) to give a compound of Formula III is carried out intially in the presence of a base, for example, sodium hydroxide, potassium hydroxide, lithium hydroxide or mixtures thereof, in a solvent, for example, N,N-dimethylformamide, water, dioxane, dimethylsulphoxide, tetrahydrofuran or mixtures followed by reaction with alkyl halides, for example, methyl iodide, ethyl iodide, allyl bromide in the presence of a base, for example, sodium bicarbonate optionally in the presence of a catalyst, for example, 18-crown-6, dibenzo-18-crown-6, trimethylbenzylammonium chloride, or tetrabutyl ammonium iodide.

The reaction of a compound of Formula III with a compound of Formula IV to give a compound of Formula V can be carried out in the presence of Mitsunobu reagents, for example, diisopropylazodicarboxylate (DIAD), dibenzylazodicarboxylate (DBAD), azodicarbonyldipiperidide (ADDP) or diethylazodicarboxylate (DEAD) in the presence of phosphines, for example, triphenyl phosphine, tributylphosphine or trimethylphosphine in a solvent, for example, tetrahydrofuran, dichloromethane, acetone, acetonitrile, dioxane, or mixtures thereof.

The coupling of a compound of Formula V with a compound of Formula VI to give a compound of Formula VII can be carried out in the presence of bis-(diphenyl-phosphino)ferrocene palladium II dichloride (Pd(dppf)Cl₂), tetrakistriphenylphosphine palladium (0) [Pd(Ph₃P)₄], palladium acetate or dichlorobistriphenylphosphine palladium (II), with a suitable base, for example, potassium carbonate, sodium acetate or potassium acetate, potassium fluoride in one or more solvent, for example, acetonitrile, dimethylformamide, toluene, tetrahydrofuran, acetone, water or dioxane.

The coupling of a compound of Formula II with a compound of Formula VI to give a compound of Formula VIII (Path B) can be carried out in the same way as the coupling of a compound of Formula V with a compound of Formula VI to give a compound of Formula VII.

The reaction of a compound of Formula VIII to give a compound of Formula IX can be carried out in a similar way as that of a compound of Formula II to give a compound of Formula III.

The reaction of a compound of Formula IX with a compound of Formula IV to give a compound of Formula VII can be carried out in the same way as reaction of a compound of Formula III with a compound of Formula IV to give a compound of Formula V.

The hydrolysis of a compound of Formula VII to give a compound of Formula X can be carried out in lithium hydroxide, potassium hydroxide or sodium hydroxide in one or more solvents, for example, tetrahydrofuran, water, methanol, dichloromethane, acetone, acetonitrile or dioxane.

The compound of Formula XI can be prepared by following Scheme II.

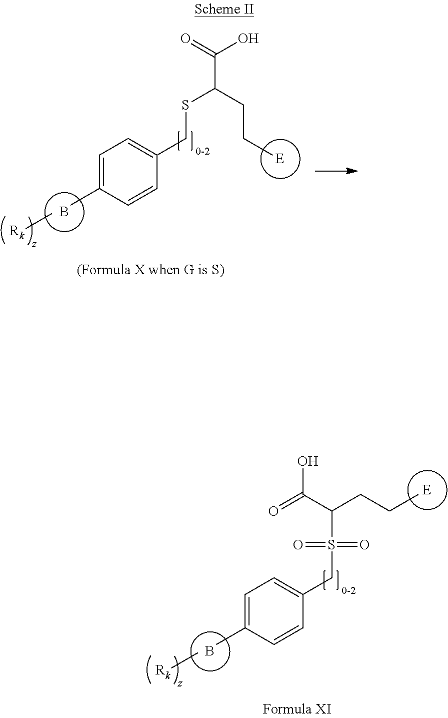

The compound of Formula X (when G is S) is oxidized to give a compound of Formula XI.

The oxidation of a compound of Formula X to give a compound of Formula XI can be carried out with an oxidizing agent, for example, metachloroperbenzoic acid or oxone in a solvent, for example, chloroform, dichoromethane, methanol, water, tetrachloromethane, or mixtures thereof.

The compound of Formula XVII can be prepared by following Scheme III.

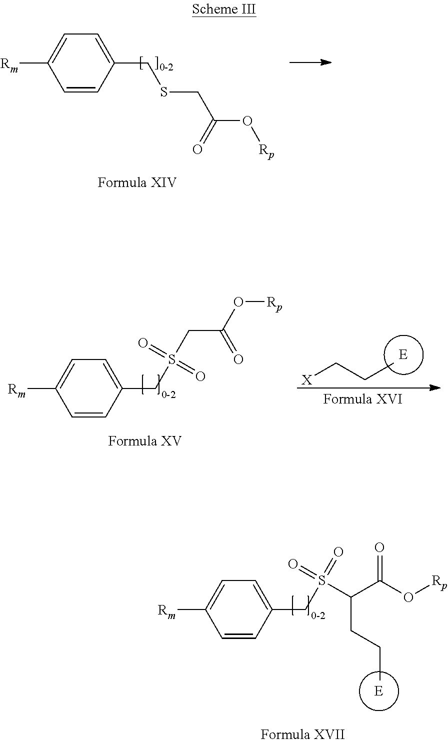

Accordingly, the compound of Formula XIV (wherein Rₘ is Br or NO₂ and Rₚ is as defined earlier) can undergo oxidation to give a compound of Formula XV. The compound of Formula XV can react with a compound of Formula XVI (wherein X is a leaving group, for example, halogen, mesylate, triflate, etc.,) to give a compound of Formula XVII.

Oxidation of a compound of Formula XIV to give a compound of Formula XV can be carried out in a similar way as the oxidation of a compound of Formula X to a compound of Formula XI.

Reaction of a compound of Formula XV with a compound of Formula XVI to give a compound of Formula XVII can be carried out in a solvent, for example, N,N-dimethylformamide, N,N-dimethylacetamide in the presence of a base, for example, potassium carbonate, sodium carbonate, triethylamine, N,N-diisopropylethylamine or mixtures thereof. The reaction can be carried out in the presence of a catalyst, for example, t-butyl ammonium iodide, t-butyl ammonium bromide, trimethylbenzylammonium chloride, benzethonium chloride, cetrimonium bromide or cetylpyridinium chloride.

The compound of Formula XXIV can be prepared by following Scheme IV.

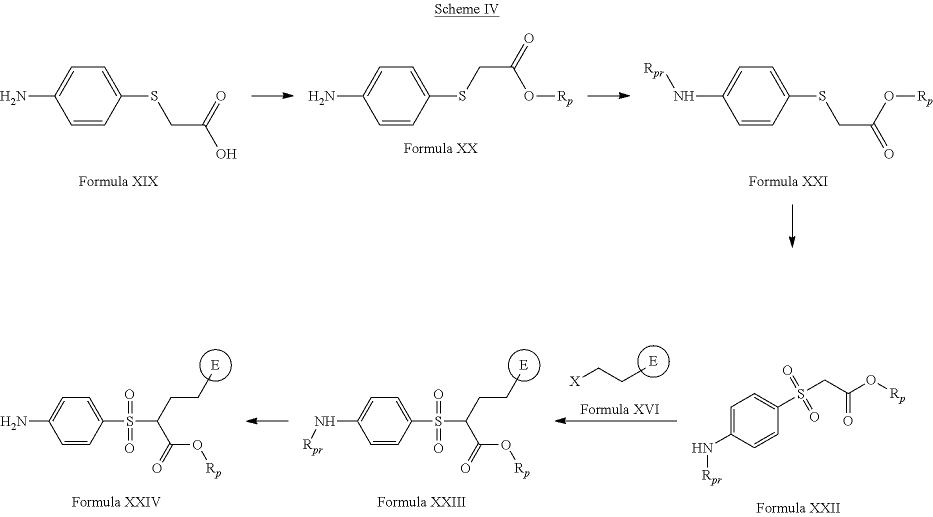

Accordingly, the compound of Formula XIX can be O-protected to give a compound of Formula XX (wherein Rₚ is as defined earlier). The compound of Formula XX can be N-protected to give a compound of Formula XXI (wherein Rₚᵣ is an amino protecting group selected from di-tert-butyl dicarbonate, t-Boc, F-moc, benzyl, tosyl or carbobenzyloxy). The compound of Formula XXI can be oxidized to give a compound of Formula XXII. The compound of Formula XXII reacts with a compound of Formula XVI (wherein X is as defined earlier) to give a compound of Formula XXIII, which then undergoes N-deprotection to give a compound of Formula XXIV.

O-protection of a compound of Formula XIX to give a compound of Formula XX can be carried out the presence of methanol and sulfuric acid.

N-protection of a compound of Formula XX to give a compound of Formula XXI can be carried out with an amino protecting group, for example, benzylchloroformate, di-tert-butyl dicarbonate, Boc anhydride or Fmoc chloride in the presence of a base, for example, triethylamine, sodium bicarbonate, N,N-diisopropylethylamine or potassium carbonate in a solvent, for example, dichloromethane, dioxane, dichloroethane, chloroform, carbon tetrachloride, t-butanol, or tetrahydrofuran.

Oxidation of a compound of Formula XXI to give a compound of Formula XXII can be carried out in a similar way as the oxidation of a compound of Formula X to a compound of Formula XI.

The reaction of a compound of Formula XXII with a compound of Formula XVI to give a compound of Formula XXIII can be carried out in a similar way as the reaction of compound of Formula XV with a compound of Formula XVI to give a compound of Formula XVII.

The N-deprotection of a compound of Formula XXIII to give a compound of Formula XXIV can be carried out in one or more solvent, for example, dichloromethane, dichloroethane, chloroform, tetrahydrofuran, water or carbon tetrachloride in the presence of an acid such as trifluoroacetic acid or hydrochloric acid.

The compounds of Formulae XXVI, XXIX, XXXII can be prepared by following Scheme V.

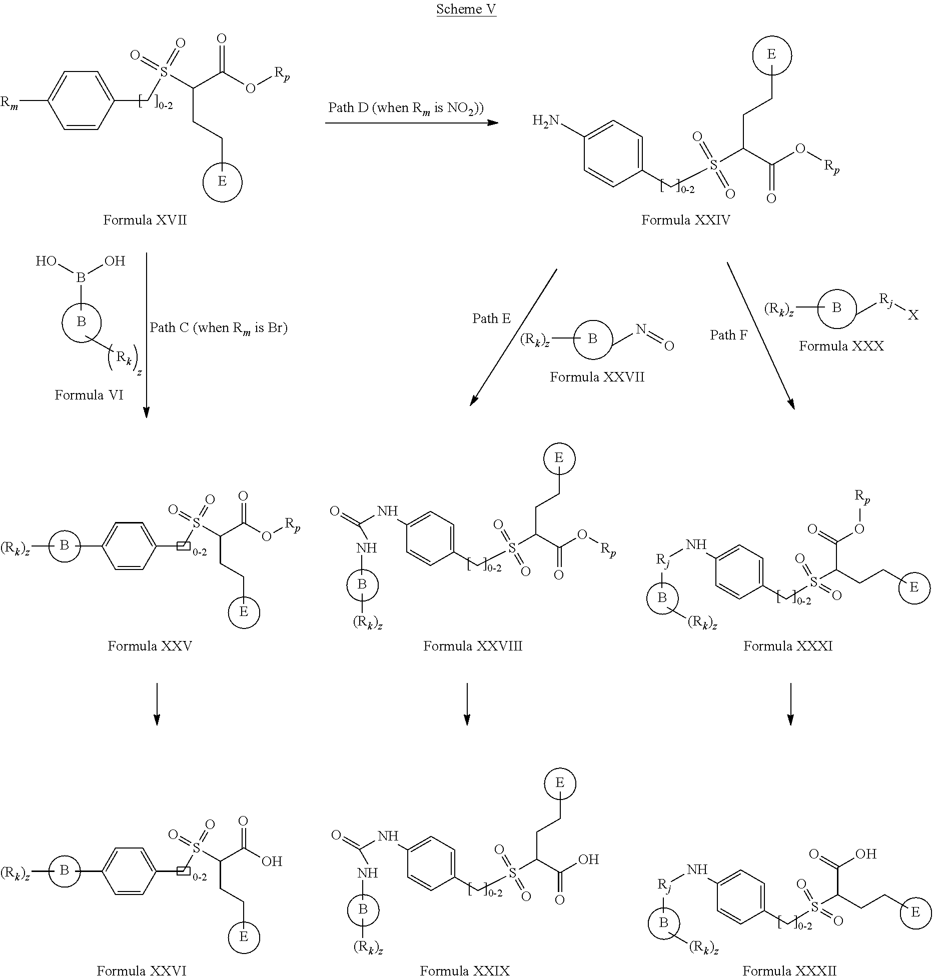

Accordingly, a compound of Formula XVII (wherein Rₘ is as defined earlier) can react through two pathways.

### Path C:

The compound of Formula XVII (when Rₘ is Br) undergoes coupling with a compound of Formula VI to give a compound of Formula XXV, which then undergoes hydrolysis to give a compound of Formula XXVI.

### Path D:

The compound of Formula XVII (when Rₘ is NO₂) undergoes reduction to give a compound of Formula XXIV.

### Path E:

The compound of Formula XXIV couples with a compound of Formula XXVII (wherein Rₖ and z are as defined earlier) to give a compound of Formula XXVIII, which can then be hydrolyzed to give a compound of Formula XXIX.

### Path F:

The compound of Formula XXIV couples with a compound of Formula XXX (wherein Rⱼ is —(CH₂)₀₋₁—CO—, —C(O)O—, —SO₂— and Rₖ and X are as defined earlier), to give a compound of Formula XXXI. The compound of Formula XXXI can then be hydrolyzed to give a compound of Formula XXXII.

Coupling of a compound of Formula XVII with a compound of Formula VI (Path C) to give a compound of Formula XXV can be carried out in the similar way as coupling of a compound of Formula V with a compound of Formula VI to give a compound of Formula VII.

Hydrolysis of a compound of Formula XXV to give a compound of Formula XXVI can be carried out in a similar way as the hydrolysis of a compound of Formula VII to a compound of Formula X.

The reduction of a compound of Formula XVII to give a compound of Formula XXIV (Path D) can be carried out in the presence of one or more reducing agent, for example, Palladium-carbon/hydrogen, Raney nickel/hydrogen, platinum/hydrogen or mixture thereof in a solvent, for example, methanol, tetrahydrofuran, ethanol, propanol, isopropanol, or mixtures thereof.

The coupling of a compound of Formula XXIV with a compound of Formula XXVII to give a compound of Formula XXVIII (Path E) can be carried out with a base, for example, triethylamine (TEA), N-methyl-morpholine (NMM), N,N-dimethylaminopyridine (DMAP) or N,N-diisopropylethylamine (DIEA) in a solvent, for example, tetrahydrofuran, dichloromethane, dimethylformamide, dioxane, acetonitrile or acetone.

Hydrolysis of a compound of Formula XXVIII to give a compound of Formula XXIX can be carried out in a similar way as the hydrolysis of a compound of Formula VII to a compound of Formula X.

Coupling of a compound of Formula XXIV with a compound of Formula XXX (Path F) to give a compound of Formula XXXI can be carried out in a similar way as the coupling of a compound of Formula XXIV with a compound of Formula XXVII to give a compound of Formula XXVIII.

Hydrolysis of a compound of Formula XXXI to give a compound of Formula XXXII can be carried out in a similar way as the hydrolysis of compound of FormulaVII to a compound of Formula X.

The compound of Formula XLI can also be prepared by following Scheme VI.

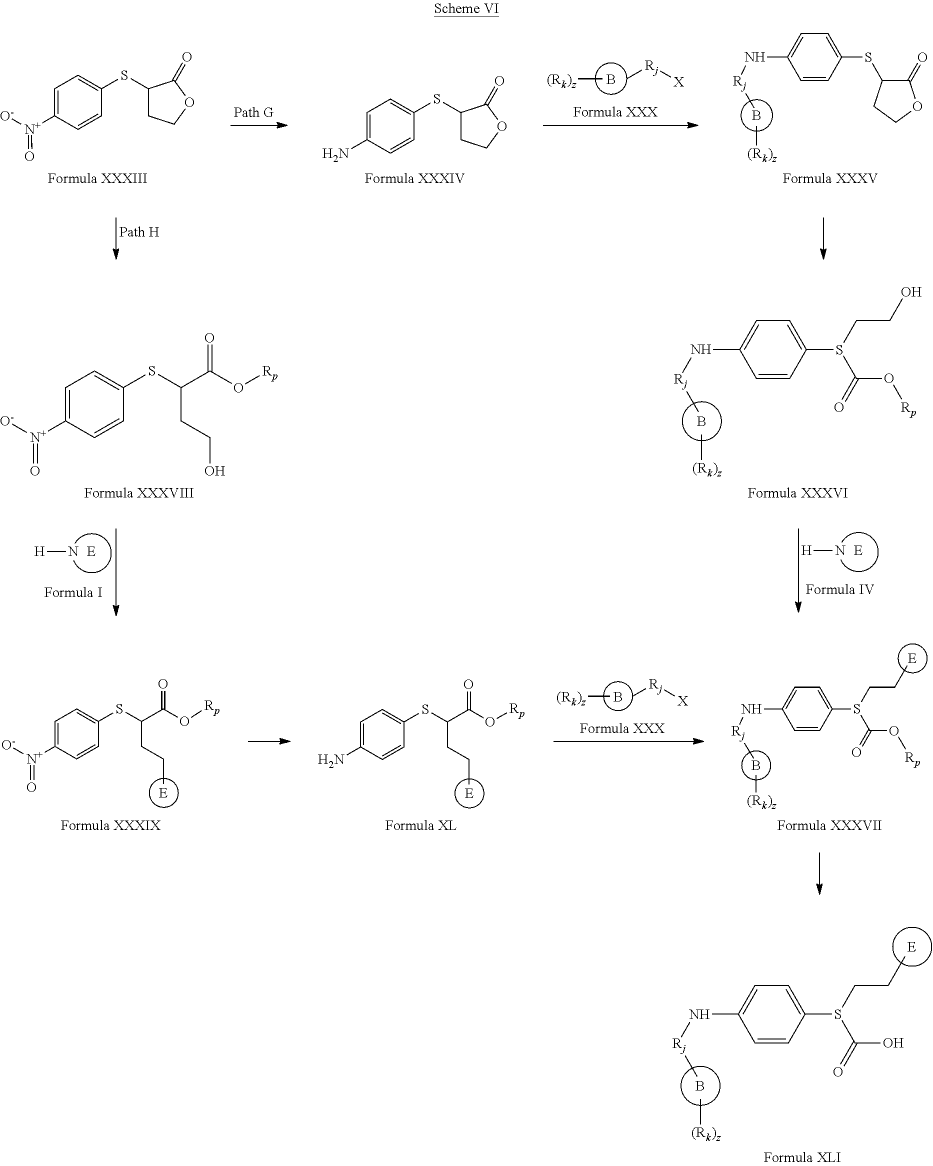

Accordingly, the compound of Formula XXXIII can react through two pathways.

### Path G:

The compound of Formula XXXIII can undergo reduction to give a compound of Formula XXXIV. The compound of Formula XXXIV can react with a compound of Formula XXX to give a compound of Formula XXXV. The compound of Formula XXXV reacts to give a compound of Formula XXXVI which undergoes reaction with a compound of Formula IV to give a compound of Formula XXXVII.

### Path H:

The compound of Formula XXXIII reacts to give a compound of Formula XXXVIII. The compound of Formula XXXVIII can react with a compound of Formula IV to give a compound of Formula XXXIX. The compound of Formula XXXIX can undergo reduction to give a compound of Formula XL. The compound of Formula XL can couple with compound of Formula XXX to give a compound of Formula XXXVII.

The compound of Formula XXXVII can then undergo hydrolysis to give a compound of Formula XLI.

The reduction of a compound of Formula XXXIII to give a compound of Formula XXXIV (Path G) can be carried out in similar way as the reduction of a compound of Formula XVII to a compound of Formula XXIV.

The coupling of a compound of Formula XXXIV with a compound of Formula XXX to give a compound of Formula XXXV can be carried out in a similar way as the coupling of compound of Formula XXIV with a compound of Formula XXVII to give a compound of Formula XXVIII.

The reaction of a compound of Formula XXXV to give a compound of Formula XXXVI can be carried out in a similar way as that of compound of Formula II to give a compound of Formula III.

The reaction of a compound of Formula XXXVI with a compound of Formula IV to give a compound of Formula XXXVII can be carried out in a similar way as the reaction of a compound of Formula III with compound of Formula IV to give a compound of Formula V.

The reaction of a compound of Formula XXXIII (Path H) to give a compound of Formula XXXVIII can be carried out in a similar way as that of a compound of Formula II to give a compound of Formula III.

The reaction of a compound of Formula XXXVIII with a compound of Formula IV to give a compound of Formula XXXIX can be carried out in a similar way as the reaction of a compound of Formula III with compound of Formula IV to give a compound of Formula V.

The reduction of a compound of Formula XXXIX to give a compound of Formula XL can be carried out in a similar way as the reduction of compound of Formula XVII to a compound of Formula XXIV.

The coupling of a compound of Formula XL with a compound of Formula XXX to give a compound of Formula XXXVII can be carried out in a similar way as the coupling of a compound of Formula XXIV to give a compound of Formula XXVIII.

The hydrolysis of a compound of Formula XXXVII to give a compound of Formula XLI can be carried out in a similar way as the hydrolysis of a compound of Formula VII to give a compound of Formula X.

The compound of Formula XXXII can also be prepared by following Scheme VII.

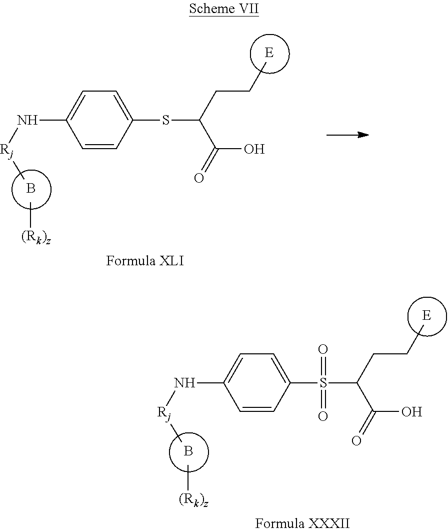

Accordingly, the compound of Formula XLI undergoes oxidation to give a compound of Formula XXXII.

The oxidation of a compound of Formula XLI to give a compound of Formula XXXII can be carried out in a similar way as the oxidation of a compound of Formula X to give a compound of Formula XI.

The compound of Formula XLV can be prepared by following Scheme VIII.

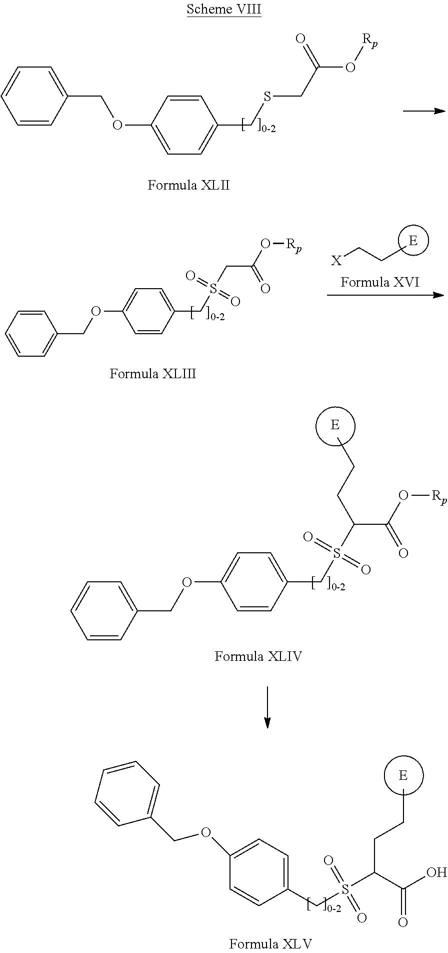

Accordingly, the compound of Formula XLII undergoes oxidation to give a compound of Formula XLIII. The compound of Formula XLIII reacts with a compound of Formula XVI to give a compound of Formula XLIV. The compound of Formula XLIV undergoes hydrolysis to give a compound of Formula XLV.

Oxidation of a compound of Formula XLII to give a compound of Formula XLIII can be carried out in the same way as the oxidation of a compound of Formula X to a compound of Formula XI.

Reaction of a compound of Formula XLIII with a compound of Formula XVI to give a compound of Formula XLIV can be carried out in the same way as the reaction of a compound of Formula XXII with a compound of Formula XVI to give a compound of Formula XXIII.

Hydrolysis of a compound of Formula XLIV to give a compound of Formula XLV can be carried out in the same way as the hydrolysis of a compound of Formula VII to give a compound of Formula X.

The compound of Formula XLVIII can be synthesized be following Scheme IX.

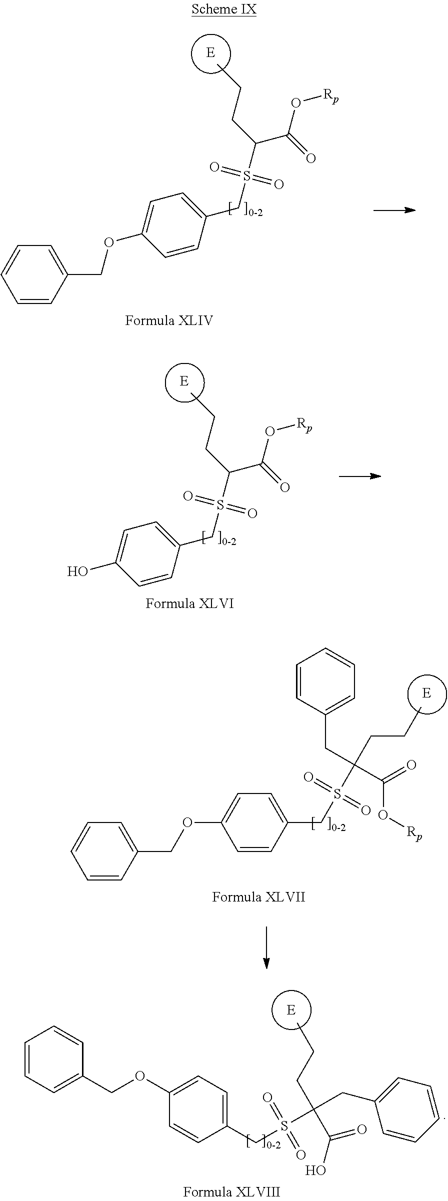

Accordingly, the compound of Formula XLIV undergoes deprotection to give a compound of Formula XLVI. The compound of Formula XLVI can be benzylated to give a compound of Formula XLVII. The compound of Formula XLVII can be hydrolyzed to give a compound of Formula XLVIII.

The deprotection of a compound of Formula XLIV to give a compound of Formula XLVI can be carried out in the presence of an acid, for example, boron trifluoride or aluminium trichloride in a solvent, for example, diethylether, dichloromethane, tetrahydrofuran, dioxane, chloroform, or mixtures thereof.

Alternatively, benzyl deprotection can be carried out (i) in hydrogenation conditions, for example, H2/Pd—C in the presence of a solvent, for example, tetrahydrofuran, ethyl acetate, methanol or mixtures thereof; or (ii) with transfer hydrogenation, for example with ammonium formate/Pd—C in a solvent, for example, methanol, ethanol, isopropanol or mixtures thereof; or (iii) with 2,3-dichloro-5,6-dicyanobenzoquinone (DDQ) in a solvent, for example, tetrahydrofuran.

Reaction of a compound of Formula XLVI to give a compound of Formula XLVII can be carried out in the presence of a base, for example, potassium carbonate, sodium hydrogen carbonate, cesium carbonate, sodium hydride, pyridine, sodium acetate, sodium thiosulfate or diisopropyl ethylamine or triethylamine in a solvent, for example, N,N-dimethylformamide, water, dioxane, dimethylsulphoxide, tetrahydrofuran or mixtures thereof.

Hydrolysis of a compound of Formula XLVII to give a compound of Formula XLVIII can be carried out in a similar way as the hydrolysis of a compound of Formula VII to give a compound of Formula X.

In the above schemes, where specific reagents, for example, bases, acids, solvents, condensing agents, hydrolyzing agents, catalysts, etc., as mentioned, it is to be understood that other reagents, e.g., other acids, bases, solvents, condensing agents, reducing agents, deprotecting agents, hydrolyzing agents, catalysts, etc., known to one of ordinary skill in the art may be used. Similarly, reaction temperatures and durations may be adjusted according to the desired needs without undue experimentation and well within the abilities of one of ordinary skill in the art.

The compounds described herein may be administered to an animal for treatment orally, topically, rectally, internasally, or by parenteral route. Pharmaceutical compositions disclosed herein comprise pharmaceutically effective amounts of compounds described herein formulated together with one or more pharmaceutically acceptable carriers, excipients or diluents.

Solid form preparations for oral administration include capsules, tablets, pills, powders, granules, lozenges, troches, cachets and suppositories. For solid form preparations, active compounds can be mixed with one or more inert, pharmaceutically acceptable excipients or carrier; tablets and capsules for oral administration may contain conventional excipients such as binding agents and/or dissolution enhancers, for example, polyvinyl pyrrolidine, cellulose, mucilage of starch, gelatin, sorbitol, syrup, acacia or tragacanth; fillers or bulking agents, for example, microcrystalline cellulose, sugar, maize-starch, calcium phosphate, sorbitol or lactose; lubricants, for example, talc, silica, polyethylene glycol, magnesium stearate or stearic acid; disintegrating agents and binder, for example, croscarmellose sodium, pregelatinized starch, sodium starch gylcollate or potato starch; glidants, for example, colloidal silicon dioxide or talc; antiadherants, for example, magnesium stearate or sodium lauryl sulfate; and coating materials.

Capsules, tablets or pills may also comprise buffering agents.

Tablets, capsules, pills or granules can be prepared using one or more coatings or shells to modulate the release of active ingredients, for example, enteric coatings or other coatings known to one of ordinary skill in the art.

### General Example

A formulation of a tablet could typically contain from 0.01 mg to 500 mg of active compound while tablet fill weight may range from 50 mg to 1000 mg. An example is illustrated below.

| Ingredients | Amount % w/w |
|---|---|
| Active Compound | 0.01 to 20 mg |
| Microcrystalline Cellulose | about 50% to about 90% |
| Croscarmellose Sodium | about 1% to about 10% |
| Pregelatinized Starch | about 1% to about 15% |
| Polyvinyl Pyrrolidone (K-30) | about 5% to about 12% |
| Talc | about 0.1% to about 2% |
| Magnesium Stearate | about 0.1% to about 2% |
| Colloidal Silicon Dioxide | about 0.1% to about 2% |

Liquid form preparations for oral administration include pharmaceutically acceptable emulsions, solutions, suspensions, syrups or elixirs. In such liquid form preparations, active compounds can be mixed with water or one or more non-toxic solvents, solubilizing agents or emulsifiers, for example, water, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil, glycerol, fatty acid esters of sorbitan or mixtures thereof. Oral compositions can also include one or more adjuvants, for example, wetting agents, emulsifying agents, suspending agents, sweetening agents, flavoring agents, perfuming agents, or mixtures thereof.

Injectable preparations, for example, sterile injections, and aqueous suspensions may be formulated according to methods known to one of ordinary skill in the art, and in particular, using one or more suitable dispersing or wetting and suspending agents. Acceptable vehicles and solvents that may be employed include one or more of water, Ringer's solution, isotonic sodium chloride, or mixtures thereof.

Suppositories for rectal administration of the compound of this invention can be prepared by mixing the drug with suitable nonirritating excipients such as cocoa butter and polyethylene glycols, which are solid at ordinary temperatures but liquid at body temperature and which therefore melt in the rectum and release the drug.

Dosage forms for topical or transdermal administration of a compound of the present invention include ointments, pastes, creams, lotions, gels, powders, solutions, sprays, inhalants or patches. Active compounds can be admixed under sterile conditions with one or more pharmaceutically acceptable carriers and optionally any preservatives or buffers as may be required. Ophthalmic formulations, eardrops, eye ointments, powders and solutions are also encompassed within the scope of this invention.

Pharmaceutical preparations may be in unit dosage form. In unit dosage form, the preparations can be subdivided into unit doses containing appropriate quantities of active components. Unit dosage forms can be packaged preparations containing discrete capsules, powders, in vials or ampules, ointments, capsules, sachets, tablets, gels, creams or any combination and number of such packaged forms.

The following examples are set forth to demonstrate general synthetic procedures for the preparation of representative compounds of the present invention. The examples are provided to illustrate a particular aspect of the disclosure and do not limit the scope of the present invention.

### EXPERIMENTAL PROCEDURES

Various solvents, for example, dimethylformamide, benzene, tetrahydrofuran, etc., were dried using various drying reagents according to procedure as described in the literature.

### Synthesis of Starting Materials

### Example A

### Synthesis of 3-[(4-bromophenyl)sulfanyl]dihydrofuran-2(3H)-one

To a solution of p-bromothiophenol (30.0 g, 0.157 mol) in dichloromethane (200 mL) under argon atmosphere at 0° C., triethyl amine (48.0 g, 0.476 mol) and then a solution of bromolactone (27.4 g, 0.1666 mol) in dichloromethane (200 mL) were added drop wise.

This reaction mixture was stirred for about 30 minutes. The reaction was worked up by adding water and extracted in dichloromethane, dried the organic layer with sodium sulphate and concentrated, purified by 60-120 silica gel column, compound eluted at 10% ethyl acetate/hexane.

Yield: 16 g

### Example B

### Synthesis of 3-(4-bromophenoxy)dihydrofuran-2(3H)-one

To a solution of 4-bromophenol (5 g, 0.028 mol) in dry N,N-dimethylformamide (50 mL), at 0° C., 60% sodium hydride (1.38 g, 0.05 mol) was added. This was stirred for about 30 minutes at about 0° C. Afterwards, \acute{α}-bromobutyrolactone (7.19 g, 0.043 mol) was added and the reaction mixture was stirred for about 2 hours at 0° C. and subsequently stirred at room temperature for about 1 hour. The reaction mixture was heated to about 100° C. for ˜4 hours. Finally, the reaction mixture was quenched with water and extracted in ethyl acetate, the organic layer was washed with brine and water and dried over anhydrous sodium sulphate; 15 g of crude product was obtained which was purified in 100-200 mesh size silica gel column chromatography by using 25% ethyl acetate-hexane as eluent to get the desired product

Yield: 8 g

Mass: 256.69 (M−1)

### Example C

### Synthesis of 3-[(4-bromobenzyl)sulfanyl]dihydrofuran-2(3H)-one

To a solution of 3-mercapto-dihydrofuran-2-one (11.1 g, 0.042 mol) in ethanol (127 mL) was added potassium carbonate (11.7 g, 0.084 mol). 4-Bromobenzylbromide (11.1 mL, 0.0423 mol) was added to this resulting solution drop wise through a period of about ten minutes. This reaction mixture was stirred at room temperature for about 2 hours.

Ethanol was removed under reduced pressure and the compound was extracted in ethyl acetate and water. This was purified using silica gel column chromatograph wherein pure compound was obtained in 6% ethyl acetate and hexane.

Yield: 9.2 g

Mass: 287.21 (M+1)

### Example D

### Synthesis of 3-[(4-bromobenzyl)oxy]dihydrofuran-2(3H)-one

Sodium hydride (1.4 g, 0.058 g) was added to a solution of 2-hydroxy-butyrolactone (5 g, 0.049 mol) in dimethylformamide (50 mL) at about 0° C. The reaction mixture was stirred at same temperature for about 15 minutes and 4-bromobenzylbromide (12.2 g, 0.049 mol) was added to it. The reaction mixture was stirred at room temperature overnight and quenched with water (50 mL), extracted with ethyl acetate. Combined organic extract was washed with water and brine, dried (Na₂SO₄) and concentrated to get crude compound, which was purified on column using 20% ethyl acetate:hexane to obtain the title compound.

Yield: 6.7 g

### Example E

### Synthesis of methyl{[2-(4-nitrophenyl)ethyl]sulfanyl}acetate

### Step 1: Synthesis of 2-(4-nitrophenyl)ethyl methanesulfonate

To a solution of 4-nitrophenyl ethanol (20 g, 0.119 mol) in dichloromethane, triethylamine (50 mL, 0.358 mol) and methane sulfonyl chloride (11.11 mL, 0.143 mol) were added at about 0° C. The reaction mixture was stirred at room temperature for about 2 hours. Subsequently, as workup, the reaction mixture was extracted in dichloromethane and washed with water and saturated brine solution. The solvent was evaporated to obtain the title compound.

Yield: 15.6 g

Mass: 246.33 (M+1)

### Step 2: Synthesis of methyl{[2-(4-nitrophenyl)ethyl]sulfanyl}acetate

In a solution of 2-(4-nitrophenyl)ethyl methanesulfonate (28 g, 0.114 mol) in methanol (571 mL), methyl thioglycolate (11.2 mL) and potassium carbonate (31.5 g) were added. This reaction mixture was stirred at room temperature for about 4 hours. After completion of reaction, the solvent was evaporated under reduced pressure and the crude reaction mixture was extracted in ethyl acetate and washed with water and brine. The organic layer was dried over anhydrous sodium sulphate and concentrated. This was purified using silica gel column chromatograph in 5% ethyl acetate-hexane to obtain the title compound.

Yield: 30 g

Mass: 255.47 (M+1)

The following intermediate was prepared using the above synthetic procedure:
Methyl[(4-nitrobenzyl)sulfanyl]acetate

### Example F

### Synthesis of methyl{[4-(benzyloxy)benzyl]sulfanyl}acetate

### Step 1: Synthesis of [4-(benzyloxy)phenyl]methanol

Potassium carbonate (40 g, 0.289 mol) and benzyl bromide (22.5 mL, 0.188 mol) were added to a solution of 4-(2-hydroxyethyl)phenol (20 g, 0.144 mol) in N,N-dimethylformamide (434 mL) at room temperature and this was stirred at −25° C. conditions for about 20 hours. After completion of reaction, the reaction mixture was extracted in ethyl acetate and washed with water and saturated brine solution. The crude compound was purified on silica gel column chromatograph with 20% ethyl acetate in hexane as elutent to give the title compound.

Yield: 24 g

Mass: 246.55 (M+18)

### Step 2: Synthesis of 4-(benzyloxy)benzyl methanesulfonate

The synthesis of the said compound was carried out following the same procedure as stated in step 1 of Example E using [4-(benzyloxy)phenyl]methanol as starting material.

Yield: 32 g

### Step 3: Synthesis of methyl{[4-(benzyloxy)benzyl]sulfanyl}acetate

The synthesis of the said compound was carried out following the similar procedure as stated in step 2 of Example E using 4-(benzyloxy)benzyl methanesulfonate as starting material.

Yield: 27 g

### Example G

### Synthesis of ethyl[(4-nitrophenyl)sulfanyl]acetate

To a solution of p-nitro thiophenol (5.0 g, 0.0322 mol) in dichloromethane (50 mL), triethyl amine (9.7 g, 0.0967 mol) was added under argon atmosphere at about 0° C. and then added a solution of ethyl bromo acetate (6.4 g, 0.0387 mol) drop wise. The reaction mixture was stirred for about 5 hours. The resultant mixture was extracted in dichloromethane, dried the organic layer with sodium sulphate and concentrated, purified on silica gel column using 10% ethyl acetate/hexane as eluent to give the title compound.

Yield: 6.5 g

### Example H

### Synthesis of ethyl[(4-bromobenzyl)sulfanyl]acetate

To a solution of ethyl-2-mercapto acetate (1 g, 0.0083 mol) in ethanol (125 ml), potassium carbonate (2.3 g, 0.016 mol) and 4-bromobenzyl bromide (2.63 g, 0.01 mol) were added at about 0° C. The reaction was stirred at room temperature for about 2 hours. Subsequently, ethanol was removed under reduced pressure and the compound was extracted in ethyl acetate, washing with water. This was purified on silica gel column chromatograph using 6% ethyl acetate/hexane as eluent to give the title compound.

Yield: 0.85 g

Mass: 287.3 (M−1)

The following intermediates were prepared by following the above synthetic procedure:
Ethyl{[2-(4-bromophenyl)ethyl]sulfanyl}acetate

Mass: 325.40 (M+Na)
Ethyl[(4-bromophenyl)sulfanyl]acetate

### Example I

### Synthesis of 3-[(4-nitrophenyl)sulfanyl]dihydrofuran-2(3H)-one

To a solution of p-nitro thiophenol (10.0 g, 0.0645 mol) in dichloromethane (75 mL) under argon atmosphere at about 0° C., was added triethyl amine (19.4 g, 0.1935 mol) and then added a solution of bromolactone (11.1 g, 0.067 mol) in dichloromethane (75 mL) drop wise. The reaction mixture was stirred for about 30 minutes. Subsequently, the crude compound was obtained by adding water to the reaction mixture and extracting in dichloromethane. The organic layer was dried with sodium sulphate and concentrated, purified by silica gel column, using 30% ethyl acetate/hexane as eluent to obtain the title compound.

Yield: 11 g

### Synthetic Procedure for Scheme I

### Example 1

### Path A

### Synthesis of 2-[(3′-fluoro-4′-methoxybiphenyl-4-yl)sulfanyl]-4-(6-methoxy-4-oxo-1,2,3-benzotriazin-3(4H)-yl)butanoic acid (Compound no. 171)

### Step 1: Synthesis of methyl 2-[(4-bromophenyl)sulfanyl]-4-hydroxybutanoate

To a solution of 3-[(4-bromophenyl)sulfanyl]dihydrofuran-2(3H)-one (10.0 g, 0.0366 mol) in dimethylformamide (40 mL) and water (10 mL), sodium hydroxide (1.75 g, 0.0439 mol) was added and the reaction mixture was stirred for about 30 minutes. To the resultant mixture, sodium bicarbonate (3.6 g, 0.043 mol), 18 crown 6 (0.96 g, 0.0036 mol) and methyl iodide (7.7 g, 0.054 mol) were added and stirred overnight. The reaction mixture was extracted in ethyl acetate, the organic layer was dried with sodium sulphate and concentrated, purified by silica gel column using 8% ethyl acetate/hexane as eluent to obtain the title compound.

Yield: 8.0 gm

### Step 2: Synthesis of methyl 2-[(4-bromophenyl)sulfanyl]-4-(7-methoxy-4-oxo-1,2,3-benzotriazin-3(4H)-yl)butanoate

To a solution of the methyl 2-[(4-bromophenyl)sulfanyl]-4-hydroxybutanoate (8 g, 0.0262 mol) in tetrahydrofuran (90 mL) under argon atmosphere, 7-methoxy-1,2,3-benzotriazin-4(3H)-one (5.5 g, 0.031 mol) and triphenyl phosphine (10.3 g, 0.039 mol) were added and cooled to about 0° C. and then DIAD (7.9 g, 0.039 mol) was added. This reaction mixture was stirred for about 30 minutes. The resultant reaction mixure was extracted in ethyl acetate, dried with sodium sulphate and concentrated, purified by silica gel column with 15% ethyl acetate/hexane to obtain the title product.

Yield: 2 g

LCMS: 465.97 (M+1)

### Step 3: Synthesis of methyl 2-[(3′-fluoro-4′-methoxybiphenyl-4-yl)sulfanyl]-4-(6-methoxy-4-oxo-1,2,3-benzotriazin-3(4H)-yl)butanoate

To a solution of methyl 2-[(4-bromophenyl)sulfanyl]-4-(7-methoxy-4-oxo-1,2,3-benzotriazin-3(4H)-yl)butanoate (0.5 g, 0.0010 mol) in dimethylformamide (10 mL) under argon atmosphere, potassium carbonate (0.446 g, 0.00323 mol) and phenyl boronic acid (0.366 g, 0.0021 mol) were added and the reaction mass was heated at about 100° C. for about 3 hours. The resultant mixture was extracted in ethyl acetate, dried the organic layer with sodium sulphate and concentrated, purified by silica gel column with 15% ethyl acetate/hexane to obtain the title product.

Yield: 0.3 g

LCMS: 510.11 (M+1)

### Step 4: Synthesis of 2-[(3′-fluoro-4′-methoxybiphenyl-4-yl)sulfanyl]-4-(6-methoxy-4-oxo-1,2,3-benzotriazin-3(4H)-yl)butanoic acid

To a solution of methyl 2-[(3′-fluoro-4′-methoxybiphenyl-4-yl)sulfanyl]-4-(6-methoxy-4-oxo-1,2,3-benzotriazin-3(4H)-yl)butanoate (0.3 g, 0.0005 mol) in tetrahydrofuran (5 mL) and methanol (5 mL), a solution of lithium hydroxide (0.037 g, 0.0008 mol) in water was added and the reaction mixture was stirred for about 1 hour. To the resultant mixture, sodium bisulphite solution was added to acidify and then extracted in ethyl acetate. The organic layer was dried with sodium sulphate and concentrated and purified by preperative TLC with 10% methanol/dichloromethane to obtain the title product.

Yield: 0.080 g

LCMS: 496.06 (M+1)

NMR (DMSO-d₆, 400 MHz)-δ 8.11-8.14 (1H, d, J=12 Hz), 7.46-7.60 (8H, d, J=8 Hz), 7.23-7.25 (1H, d, J=8 Hz), 4.52 (2H, m), 3.87-3.97 (6H, m), 3.3 (1H, s), 2.34 (1H, m), 2.18 (1H, m).

The following compounds were prepared by following the above synthetic route
2-[(3′-Fluoro-4′-methoxybiphenyl-4-yl)sulfanyl]-4-[4-oxo-7-(trifluoromethyl)-1,2,3-benzotriazin-3(4H)-yl]butanoic acid (Compound no. 75)

Mass: 551.03 (M+NH4+)
2-[(3′-Fluoro-4′-methoxybiphenyl-4-yl)sulfanyl]-4-(7-methyl-4-oxo-1,2,3-benzotriazin-3(4H)-yl)butanoic acid (Compound no. 105)

Mass: 480.22
2-[(4′-Chlorobiphenyl-4-yl)sulfanyl]-4-(6-methoxy-4-oxo-1,2,3-benzotriazin-3(4H)-yl)butanoic acid (Compound no. 106)

Mass: 482.13
2-[(4′-Chlorobiphenyl-4-yl)sulfanyl]-4-(6-methyl-4-oxo-1,2,3-benzotriazin-3(4H)-yl)butanoic acid (Compound no. 107)

Mass: 466.20
2-[(3′-Fluoro-4′-methoxybiphenyl-4-yl)sulfanyl]-4-(6-methyl-4-oxo-1,2,3-benzotriazin-3(4H)-yl)butanoic acid (Compound no. 108)

Mass: 480.22
2-[(3′-Fluoro-4′-methoxybiphenyl-4-yl)sulfanyl]-4-(4-oxo-1,2,3-benzotriazin-3(4H)-yl)butanoic acid (Compound no. 109)

Mass: 465.75 (M+1)
4-(6-Methyl-4-oxo-1,2,3-benzotriazin-3(4H)-yl)-2-{[4′-(trifluoromethyl)biphenyl-4-yl]sulfanyl}butanoic acid (Compound no. 110)

Mass: 500.22
2-[(3′,4′-Dichlorobiphenyl-4-yl)sulfanyl]-4-(6-methyl-4-oxo-1,2,3-benzotriazin-3(4H)-yl)butanoic acid (Compound no. 111)

Mass: 502.17 (M+2)
2-[(4′-Methoxy-3′-methylbiphenyl-4-yl)sulfanyl]-4-(6-methyl-4-oxo-1,2,3-benzotriazin-3(4H)-yl)butanoic acid (Compound no. 112)

Mass: 476.28
2-[(3′,4′-Difluorobiphenyl-4-yl)sulfanyl]-4-(6-methyl-4-oxo-1,2,3-benzotriazin-3(4H)-yl)butanoic acid (Compound no. 113)

Mass: 468.22
2-[(3′-Fluoro-4′-methylbiphenyl-4-yl)sulfanyl]-4-(6-methyl-4-oxo-1,2,3-benzotriazin-3(4H)-yl)butanoic acid (Compound no. 114)

Mass: 464.25
2-[(4′-Florobiphenyl-4-yl)sulfanyl]-4-(6-methyl-4-oxo-1,2,3-benzotriazin-3(4H)-yl)butanoic acid (Compound no. 115)

Mass: 450.20
2-[(4′-Ethylbiphenyl-4-yl)sulfanyl]-4-(6-methyl-4-oxo-1,2,3-benzotriazin-3(4H)-yl)butanoic acid (Compound no. 116)

Mass: 460.22
2-[(4′-Methoxybiphenyl-4-yl)sulfanyl]-4-(6-methyl-4-oxo-1,2,3-benzotriazin-3(4H)-yl)butanoic acid (Compound no. 117)

Mass: 462.23
2-[(4′-Fluoro-3′-methylbiphenyl-4-yl)sulfanyl]-4-(6-methyl-4-oxo-1,2,3-benzotriazin-3(4H)-yl)butanoic acid (Compound no. 118)

Mass: 464.25
4-(4-Oxo-1,2,3-benzotriazin-3(4H)-yl)-2-{[4′-(trifluoromethyl)biphenyl-4-yl]sulfanyl}butanoic acid (Compound no. 119)

Mass: 486.22
4-(8-Methyl-4-oxo-1,2,3-benzotriazin-3(4H)-yl)-2-{[4′-(trifluoromethoxy)biphenyl-4-yl]sulfanyl}butanoic acid (Compound no. 120)

Mass: 516.19
4-(7-Methyl-4-oxo-1,2,3-benzotriazin-3(4H)-yl)-2-{[4′-(trifluoromethoxy)biphenyl-4-yl]sulfanyl}butanoic acid (Compound no. 121)

Mass: 516.29 (M+1)
4-(6-Methoxy-4-oxo-1,2,3-benzotriazin-3(4H)-yl)-2-{[4′-(trifluoromethoxy)biphenyl-4-yl]sulfanyl}butanoic acid (Compound no. 122)

Mass: 532.20
4-(7-Methoxy-4-oxo-1,2,3-benzotriazin-3(4H)-yl)-2-{[4′-(trifluoromethoxy)biphenyl-4-yl]sulfanyl}butanoic acid (Compound no. 123)

Mass: 532.20
2-[(3′,4′-Difluorobiphenyl-4-yl)sulfanyl]-4-(6-methoxy-4-oxo-1,2,3-benzotriazin-3(4H)-yl)butanoic acid (Compound no. 124)

Mass: 484.17
2-[(3′-Fluoro-4′-methylbiphenyl-4-yl)sulfanyl]-4-(6-methoxy-4-oxo-1,2,3-benzotriazin-3(4H)-yl)butanoic acid (Compound no. 125)

Mass: 480.25
2-[(4′-Fluoro-3′-methylbiphenyl-4-yl)sulfanyl]-4-(6-methoxy-4-oxo-1,2,3-benzotriazin-3(4H)-yl)butanoic acid (Compound no. 126)

Mass: 480.16
2-[(3′,4′-Dichlorobiphenyl-4-yl)sulfanyl]-4-(6-methoxy-4-oxo-1,2,3-benzotriazin-3(4H)-yl)butanoic acid (Compound no. 127)

Mass: 518.14
2-[(3′-Fluoro-4′-methoxybiphenyl-4-yl)sulfanyl]-4-(6-methoxy-4-oxo-1,2,3-benzotriazin-3(4H)-yl)butanoic acid (Compound no. 128)

Mass: 496.20
2-{[4′-Chloro-3′-(trifluoromethyl)biphenyl-4-yl]sulfanyl}-4-(6-methoxy-4-oxo-1,2,3-benzotriazin-3(4H)-yl)butanoic acid (Compound no. 129)

Mass: 550.12
2-[(4′-Ethylbiphenyl-4-yl)sulfanyl]-4-(6-methoxy-4-oxo-1,2,3-benzotriazin-3(4H)-yl)butanoic acid (Compound no. 130)

Mass: 476.22
2-[(3′,4′-Dimethylbiphenyl-4-yl)sulfanyl]-4-(6-methoxy-4-oxo-1,2,3-benzotriazin-3(4H)-yl)butanoic acid (Compound no. 131)

Mass: 476.22
4-(6-Methoxy-4-oxo-1,2,3-benzotriazin-3(4H)-yl)-2-[(4′-methylbiphenyl-4-yl)sulfanyl]butanoic acid (Compound no. 132)

Mass: 462.11
4-(6-Methoxy-4-oxo-1,2,3-benzotriazin-3(4H)-yl)-2-{[4′-(trifluoromethyl)biphenyl-4-yl]sulfanyl}butanoic acid (Compound no. 133)

Mass: 515.99
2-[(4′-Ethylbiphenyl-4-yl)sulfanyl]-4-(8-methyl-4-oxo-1,2,3-benzotriazin-3(4H)-yl)butanoic acid (Compound no. 134)

Mass: 460.30
2-[(3′-Fluoro-4′-methylbiphenyl-4-yl)sulfanyl]-4-(8-methyl-4-oxo-1,2,3-benzotriazin-3(4H)-yl)butanoic acid (Compound no. 135)
2-{[4-(6-Methoxypyridin-3-yl)phenyl]sulfanyl}-4-(8-methyl-4-oxo-1,2,3-benzotriazin-3(4H)-yl)butanoic acid (Compound no. 136)

Mass: 463.23
2-[(4′-Methylbiphenyl-4-yl)sulfanyl]-4-(8-methyl-4-oxo-1,2,3-benzotriazin-3(4H)-yl)butanoic acid (Compound no. 137)

Mass: 446.28
2-[(4′-Chlorobiphenyl-4-yl)sulfanyl]-4-(8-methyl-4-oxo-1,2,3-benzotriazin-3(4H)-yl)butanoic acid (Compound no. 138)

Mass: 466.25
2-[(4′-Fluorobiphenyl-4-yl)sulfanyl]-4-(8-methyl-4-oxo-1,2,3-benzotriazin-3(4H)-yl)butanoic acid (Compound no. 139)

Mass: 450.42
2-[(4′-Fluoro-3′-methylbiphenyl-4-yl)sulfanyl]-4-(8-methyl-4-oxo-1,2,3-benzotriazin-3(4H)-yl)butanoic acid (Compound no. 140)

Mass: 464.39
2-[(3′,4′-Difluorobiphenyl-4-yl)sulfanyl]-4-(8-methyl-4-oxo-1,2,3-benzotriazin-3(4H)-yl)butanoic acid (Compound no. 141)

Mass: 468.38
2-[(3′,4′-Dimethoxybiphenyl-4-yl)sulfanyl]-4-(8-methyl-4-oxo-1,2,3-benzotriazin-3(4H)-yl)butanoic acid (Compound no. 142)

Mass: 492.40
2-[(3′,4′-Dimethylbiphenyl-4-yl)sulfanyl]-4-(8-methyl-4-oxo-1,2,3-benzotriazin-3(4H)-yl)butanoic acid (Compound no. 143)

Mass: 460.55

4-(8-Methyl-4-oxo-1,2,3-benzotriazin-3(4H)-yl)-2-{[4′-(trifluoromethyl)biphenyl-4-yl]sulfanyl}butanoic acid (Compound no. 144)

Mass: 499.39
2-[(3′-Fluoro-4′-methoxybiphenyl-4-yl)sulfanyl]-4-(8-methyl-4-oxo-1,2,3-benzotriazin-3(4H)-yl)butanoic acid (Compound no. 145)

Mass: 515-96 (M+K)
4-(5-Chloro-4-oxo-1,2,3-benzotriazin-3(4H)-yl)-2-[(3′-fluoro-4′-methoxybiphenyl-4-yl)sulfanyl]butanoic acid (Compound no. 146)

Mass: 499.55 (M+1)
4-(7-Chloro-4-oxo-1,2,3-benzotriazin-3(4H)-yl)-2-[(3′-fluoro-4′-methoxybiphenyl-4-yl)sulfanyl]butanoic acid (Compound no. 147)

Mass: 499.75 (M+1)
2-{[4-(6-Methoxypyridin-3-yl)phenyl]sulfanyl}-4-(4-oxo-1,2,3-benzotriazin-3(4H)-yl)butanoic acid (Compound no. 148)

Mass: 449.20 (M+1)
2-[(4′-Methoxybiphenyl-4-yl)sulfanyl]-4-(4-oxo-1,2,3-benzotriazin-3(4H)-yl)butanoic acid (Compound no. 149)

Mass: 448.25 (M+1)
2-[(3′-Fluoro-4′-methylbiphenyl-4-yl)sulfanyl]-4-(4-oxo-1,2,3-benzotriazin-3(4H)-yl)butanoic acid (Compound no. 150)

Mass: 450.27 (M+1)
2-[(3′,4′-Difluorobiphenyl-4-yl)sulfanyl]-4-(4-oxo-1,2,3-benzotriazin-3(4H)-yl)butanoic acid (Compound no. 151)

Mass: 454.26 (M+1)
2-[(3′-Methoxybiphenyl-4-yl)sulfanyl]-4-(4-oxo-1,2,3-benzotriazin-3(4H)-yl)butanoic acid (Compound no. 152)

Mass: 448.31 (M+1)
2-[(4′-Fluorobiphenyl-4-yl)sulfanyl]-4-(4-oxo-1,2,3-benzotriazin-3(4H)-yl)butanoic acid (Compound no. 153)

Mass: 436.29 (M+1)
2-(Biphenyl-4-yl)sulfanyl-4-(4-oxo-1,2,3-benzotriazin-3(4H)-yl)butanoic acid (Compound no. 154)

Mass: 418.26 (M+1)
2-[(2′,3′-Difluorobiphenyl-4-yl)sulfanyl]-4-(4-oxo-1,2,3-benzotriazin-3(4H)-yl)butanoic acid (Compound no. 155)

Mass: 454.26 (M+1)
2-[(4′-tert-Butylbiphenyl-4-yl)sulfanyl]-4-(4-oxo-1,2,3-benzotriazin-3 (4H)-yl)butanoic acid (Compound no. 166)

Mass: 474.26 (M+1)
2-[(4′-Ethylbiphenyl-4-yl)sulfanyl]-4-(4-oxo-1,2,3-benzotriazin-3(4H)-yl)butanoic acid (Compound no. 167)

Mass: 446.23 (M+1)
4-(4-Oxo-1,2,3-benzotriazin-3(4H)-yl)-2-{[4′-(propan-2-yl)biphenyl-4-yl]sulfanyl}butanoic acid (Compound no. 168)

Mass: 460.28 (M+1)
4-(4-Oxo-1,2,3-benzotriazin-3(4H)-yl)-2-{[4′-(trifluoromethyl)biphenyl-4-yl]sulfanyl}butanoic acid (Compound no. 169)

Mass: 486.15 (M+1)
4-(4-Oxo-1,2,3-benzotriazin-3(4H)-yl)-2-{[4′-(trifluoromethoxy)biphenyl-4-yl]sulfanyl}butanoic acid (Compound no. 170)

Mass: 486.22 (M+1)
4-(7-Methoxy-4-oxo-1,2,3-benzotriazin-3(4H)-yl)-2-{[4-(6-methoxypyridin-3-yl)phenyl]sulfanyl}butanoic acid (Compound no. 172)

Mass: 479.18
4-(7-Methoxy-4-oxo-1,2,3-benzotriazin-3(4H)-yl)-2-{[4′-(trifluoromethyl)biphenyl-4-yl]sulfanyl}butanoic acid (Compound no. 173)

Mass: 516.28
2-[(4′-Methoxybiphenyl-4-yl)sulfanyl]-4-(7-methoxy-4-oxo-1,2,3-benzotriazin-3(4H)-yl)butanoic acid (Compound no. 174)

Mass: 478.31
2-[(3′-Fluoro-4′-methylbiphenyl-4-yl)sulfanyl]-4-(7-methoxy-4-oxo-1,2,3-benzotriazin-3(4H)-yl)butanoic acid (Compound no. 175)

Mass: 480.34
4-(7-Methoxy-4-oxo-1,2,3-benzotriazin-3(4H)-yl)-2-{[4-(1-methyl-1H-pyrazol-4-yl)phenyl]sulfanyl}butanoic acid (Compound no. 176)

Mass: 452.19
2-[(3′,4′-Dimethoxybiphenyl-4-yl)sulfanyl]-4-(7-methoxy-4-oxo-1,2,3-benzotriazin-3(4H)-yl)butanoic acid (Compound no. 177)

Mass: 508.36
2-[(4′-Ethylbiphenyl-4-yl)sulfanyl]-4-(7-methoxy-4-oxo-1,2,3-benzotriazin-3(4H)-yl)butanoic acid (Compound no. 178)

Mass: 476.19
2-[(4′-Fluoro-3′-methylbiphenyl-4-yl)sulfanyl]-4-(7-methoxy-4-oxo-1,2,3-benzotriazin-3(4H)-yl)butanoic acid (Compound no. 179)

Mass: 480.31
2-[(4′-Chlorobiphenyl-4-yl)sulfanyl]-4-(7-methoxy-4-oxo-1,2,3-benzotriazin-3(4H)-yl)butanoic acid (Compound no. 180)

Mass: 482.18
2-[(3′,4′-Dichlorobiphenyl-4-yl)sulfanyl]-4-(7-methoxy-4-oxo-1,2,3-benzotriazin-3(4H)-yl)butanoic acid (Compound no. 181)

Mass: 516.06
2-[(4′-Chlorobiphenyl-4-yl)oxy]-4-(4-oxo-1,2,3-benzotriazin-3(4H)-yl)butanoic acid (Compound no. 211)

Mass: 436.26
2-[(4′-Chlorobiphenyl-4-yl)methoxy]-4-(6-fluoro-4-oxo-1,2,3-benzotriazin-3(4H)-yl)butanoic acid (Compound no. 212)

Mass: 468.48 (M−1)
2-[(4′-Chlorobiphenyl-4-yl)oxy]-4-(7-chloro-4-oxo-1,2,3-benzotriazin-3(4H)-yl)butanoic acid (Compound no. 213)

Mass: 470.21 & 472.16 (M+1) (Cl=35 or Cl=37)
2-[(4′-Chlorobiphenyl-4-yl)oxy]-4-[4-oxo-7-(trifluoromethyl)-1,2,3-benzotriazin-3(4H)-yl]butanoic acid (Compound no. 214)

Mass: 504.29
2-[(4′-Chlorobiphenyl-4-yl)oxy]-4-(5-fluoro-4-oxo-1,2,3-benzotriazin-3(4H)-yl)butanoic acid (Compound no. 215)

Mass: 454.21
2-[(4′-Chlorobiphenyl-4-yl)oxy]-4-(5-chloro-4-oxo-1,2,3-benzotriazin-3(4H)-yl)butanoic acid (Compound no. 216)

Mass: 472.22
2-[(4′-Chlorobiphenyl-4-yl)oxy]-4-(6-methoxy-4-oxo-1,2,3-benzotriazin-3(4H)-yl)butanoic acid (Compound no. 217)

Mass: 466.24
2-[(4′-Chlorobiphenyl-4-yl)oxy]-4-(5-methyl-4-oxo-1,2,3-benzotriazin-3(4H)-yl)butanoic acid (Compound no. 218)

Mass: 450 (M+1)
(2R)-2-[(4′-Chlorobiphenyl-4-yl)oxy]-4-(6-fluoro-4-oxo-1,2,3-benzotriazin-3(4H)-yl)butanoic acid (Compound no. 219)

Mass: 454.21
(2R)-2-[(4′-Chlorobiphenyl-4-yl)oxy]-4-(7-chloro-4-oxo-1,2,3-benzotriazin-3(4H)-yl)butanoic acid (Compound no. 220)

Mass: 472.16
(2R)-2-[(4′-Chlorobiphenyl-4-yl)oxy]-4-(6-methoxy-4-oxo-1,2,3-benzotriazin-3(4H)-yl)butanoic acid (Compound no. 221)

Mass: 466.31
(2R)-2-[(4′-Chlorobiphenyl-4-yl)oxy]-4-(6,7-difluoro-4-oxo-1,2,3-benzotriazin-3(4H)-yl)butanoic acid (Compound no. 222)

Mass: 470.41 (ES −ve)
2-[(4′-Chlorobiphenyl-4-yl)methoxy]-4-(7-chloro-4-oxo-1,2,3-benzotriazin-3(4H)-yl)butanoic acid (Compound no. 223)

Mass: 484.19
2-[(4′-Chlorobiphenyl-4-yl)methoxy]-4-(7-methoxy-4-oxo-1,2,3-benzotriazin-3(4H)-yl)butanoic acid (Compound no. 224)

Mass: 478.51 (ES −ve)
(2R)-2-[(4′-Chlorobiphenyl-4-yl)oxy]-4-(2,4-dioxo-2H-1,3-benzoxazin-3(4H)-yl)butanoic acid (Compound no. 225)

Mass: 452.19
(2R)-2-[(4′-Chlorobiphenyl-4-yl)oxy]-4-(1-oxophthalazin-2(1H)-yl)butanoic acid (Compound no. 226)

Mass: 435.25

### Path B

### Example 2

### Synthesis of 2-{[(4′-chlorobiphenyl-4-yl)methyl]sulfanyl}-4-(4-oxo-1,2,3-benzotriazin-3(4H)-yl)butanoic acid (Compound no. 156)

### Step 1: Synthesis of 3-{[(4′-chlorobiphenyl-4-yl)methyl]sulfanyl}dihydrofuran-2(3H)-one

To a solution of 3-[(4-bromobenzyl)sulfanyl]dihydrofuran-2(3H)-one (1.5 g, 0.00522 mmol) in N,N-dimethylformamide (26 mL), potassium carbonate (2.16 g, 0.0156 mol) was added. To this mixture, 4-chlorobenzeneboronic acid (1.63 g, 1.0104 mol) and tetrakis triphenylphosphine palladium (0) (0.6 g, 0.522 mmol) were added. The resulting solution was heated at about 110° C. for about 6 hours. Further, the reaction mixture was cooled to room temperature by added water and extracted twice in ethyl acetate (40 mL). The organic layer was separated and dried under sodium sulphate and concentrated in reduced pressure. Purification of the crude compound was performed through 60-120 mesh size silica gel column chromatograph with 20% ethyl acetate-hexane to obtain the title compound. (Yield: 2.1 g)

### Step 2: Synthesis of (2-{[(4′-chlorobiphenyl-4-yl)methyl]sulfanyl}-4-hydroxy butanoyl)sodium

To a solution of 3-{[(4′-chlorobiphenyl-4-yl)methyl]sulfanyl}dihydrofuran-2(3H)-one (2 g, 0.00628 mol) in N,N′-dimethylformamide:water (15 mL:4 mL) was added sodium hydroxide (0.327 g, 0.00817 mol) at 0° C. The reaction mixture was stirred at room temperature for 2 hours. This reaction mixture was directly taken for next step without any work up.

### Step 3: Synthesis of 2-(4′-chloro-biphenyl-4-ylmethylsulfanyl)-4-hydroxy-butyric acid allyl ester

To a solution of (2-{[(4′-chlorobiphenyl-4-yl)methyl]sulfanyl}-4-hydroxy butanoyl)sodium (2 g, 0.00628 mol) in N,N′-dimethylformamide:water (15 mL:4 mL) was added sodium bicarbonate (634 mg, 0.00075 mol), 18-Crown-6 (0.166 g, 0.628 mmol) and allyl bromide (0.815 mL, 0.00943 mol) and stirred overnight at room temperature. This was extracted in ethyl acetate and washed with water and taken directly for next step without purification.

Yield: 1.2 g

MS −375.41 (M−1)

### Step 4: Synthesis of prop-2-en-1-yl-2-{[(4′-chlorobiphenyl-4-yl)methyl]sulfanyl}-4-(4-oxo-1,2,3-benzotriazin-3(4H)-yl)butanoate

To a solution of 2-(4′-chloro-biphenyl-4-ylmethylsulfanyl)-4-hydroxy-butyric acid allyl ester (0.216 g, 0.574 mmol) in tetrahydrofuran (5 mL), triphenyl phosphine (0.301 g, 0.0011 mol) and benzotriazinone (0.101 g, 0.689 mmol) were added at room temperature. At ˜0° C., diisopropyl azodicarboxylate (0.174 mL, 0.00088 mol) was added to it. This reaction mixture was stirred at room temperature for about one hour. After completion, the reaction mixture was concentrated under reduced pressure and was directly purified through silica gel column chromatograph with 10% ethyl acetate in hexane to obtain the title compound.

Yield: 110 mg

### Step 5: Synthesis of 2-{[(4′-chlorobiphenyl-4-yl)methyl]sulfanyl}-4-(4-oxo-1,2,3-benzotriazin-3(4H)-yl)butanoic acid

To a solution of prop-2-en-1-yl 2-{[(4′-chlorobiphenyl-4-yl)methyl]sulfanyl}-4-(4-oxo-1,2,3-benzotriazin-3(4H)-yl)butanoate (0.17 g, 0.309 mmol) in acetonitrile (2 mL), morpholine (0.269 mL, 3.09 mmol) and tetrakis triphenylphosphine palladium(0) (0.035 g, 9 mmol) were added at room temperature. This was stirred at room temperature for about one hour. Acetonitrile was removed under reduced pressure; the compound was extracted in ethyl acetate to remove impurities. Aqueous layer was acidified with sodium bisulphate and the pure title compound was extracted in ethyl acetate.

Yield: 121 mg

MS −478.49 (M−1)

NMR (MeOD, 400 MHz): 6.227 (1H, d, J=8 Hz), 8.042 (1H, d, J=8 Hz) 7.921 (1H, t, J=6.8 Hz), 7.781 (1H, t, J=8 Hz), 7.55-7.32 (8H, m) 4.52 (2H, t, J=6.8 Hz), 3.93-3.84 (2H, m), 3.35-3.25 (3H, m), 2.44 (1H, dd, J=6.8 Hz), 2.14 (1H, dd, J=6.4 Hz).

The following compounds were prepared using the above synthetic procedure:
2-{[(4′-Chlorobiphenyl-4-yl)methyl]sulfanyl}-4-(7-methyl-4-oxo-1,2,3-benzotriazin-3(4H)-yl)butanoic acid (Compound no. 157)

Mass: 478.49 (M−1)
2-{[(4′-Chlorobiphenyl-4-yl)methyl]sulfanyl}-4-(7-methoxy-4-oxo-1,2,3-benzotriazin-3(4H)-yl)butanoic acid (Compound no. 158)

Mass: 494.4 (M−1)
2-{[(4′-Chlorobiphenyl-4-yl)methyl]sulfanyl}-4-(6-fluoro-4-oxo-1,2,3-benzotriazin-3(4H)-yl)butanoic acid (Compound no. 159)

Mass: 482.45 (M−1)
2-{[(4′-Chlorobiphenyl-4-yl)methyl]sulfanyl}-4-(8-methyl-4-oxo-1,2,3-benzotriazin-3(4H)-yl)butanoic acid (Compound no. 160)

Mass: 478.63 (M−1)
2-{[(4′-Chlorobiphenyl-4-yl)methyl]sulfanyl}-4-[4-oxo-7-(trifluoromethyl)-1,2,3-benzotriazin-3(4H)-yl]butanoic acid (Compound no. 161)

Mass: 532.47 (M−1)
2-{[(4′-Chlorobiphenyl-4-yl)methyl]sulfanyl}-4-(5-chloro-4-oxo-1,2,3-benzotriazin-3(4H)-yl)butanoic acid (Compound no. 162)

Mass: 500.24 (M−1)
2-[(4′-Chlorobiphenyl-4-yl)methoxy]-4-(4-oxo-1,2,3-benzotriazin-3(4H)-yl)butanoic acid (Compound no. 227)

Mass: 448.55 (ES −ve)
2-[(4′-Chlorobiphenyl-4-yl)methoxy]-4-(1-oxophthalazin-2(1H)-yl)butanoic acid (Compound no. 228)

Mass: 447.61 (ES −ve)
2-[(4′-Chlorobiphenyl-4-yl)methoxy]-4-(2,4-dioxo-2H-1,3-benzoxazin-3(4H)-yl)butanoic acid (Compound no. 229)

Mass: 464.53 (ES −ve)

### Example 3

### Synthesis of 2-{[2-(4′-chlorobiphenyl-4-yl)ethyl]sulfanyl}-4-(6-fluoro-4-oxo-1,2,3-benzotriazin-3(4H)-yl)butanoic acid (Compound no. 195)

### Step 1: Synthesis of 3-{[2-(4′-chlorobiphenyl-4-yl)ethyl]sulfanyl}dihydrofuran-2(3H)-one

3-{[2-(4-Bromophenyl)ethyl]sulfanyl}dihydrofuran-2(3H)-one (5 g, 16.6 mmol) was dissolved in N,N-dimethylformamide (50 mL), and potassium carbonate (6.88 g, 49.8 mmol) was added to it. To this mixture, 4-chlorobenzeneboronic acid (5.18 g, 33.2 mmol) and tetrakis triphenylphosphine palladium (0) (1.9 g, 1.66 mmol) were added to it. The reaction mixture was heated at about 110° C. for ˜6 hours. Subsequently, it was cooled to room temperature, added water and extracted in ethyl acetate (40 mL). The organic layer was separated and dried under sodium sulphate and concentrated in reduced pressure. Purification of the crude compound was performed through 60-120 mesh size silica gel column chromatograph using 20% ethyl acetate-hexane as eluent.

Yield: 2.1 g

Mass: 355.18 (M+Na)

### Step 2: Synthesis of methyl 2-{[2-(4′-chlorobiphenyl-4-yl)ethyl]sulfanyl}-4-hydroxybutanoate

To a solution of 3-{[2-(4′-chlorobiphenyl-4-yl)ethyl]sulfanyl}dihydrofuran-2(3H)-one (3.7 g, 0.011 mmol) in methanol:water (40 mL:5 mL), sodium hydroxide (0.445 g, 0.011 mmol) was added at 0° C. This was stirred at room temperature for about 2 hours. This reaction was taken directly for next step without any work up.

To a solution of (2-{[2-(4′-chlorobiphenyl-4-yl)ethyl]sulfanyl}-4-hydroxybutanoyl)sodium (3.7 g, 0.011 mmol) in N,N-Dimethylformamide:water (15 mL:4 mL) was added sodium bicarbonate (0.936 g, 0.011 mmol), methyl iodide (2.15 mL, 0.0334 mmol) and stirred overnight at room temperature. This was extracted in ethyl acetate and washed with water and taken directly for next step without purification.

Yield: 1.3 g (crude).

MS: 363.28 (M−1)

### Step 3: Synthesis of 2-[2-(4′-chloro-biphenyl-4-yl)-ethylsulfanyl]-4-(6-fluoro-4-oxo-4H-benzo[d][1,2,3]triazin-3-yl)-butyric acid methyl ester

To a solution of 2-[2-(4′-chloro-biphenyl-4-yl)-ethylsulfanyl]-4-hydroxy-butyric acid methyl ester (0.4 g, 0.00109 mol) in tetrahydrofuran (5 mL), triphenyl phosphine (0.574 g, 0.00219 mol) and 6-fluoro benzotriazinone (0.216 g, 0.00131 mol) were added at room temperature. At 0° C., diisopropyl azodicarboxylate (0.332 mL, 0.0016 mol) was added to it. This reaction mixture was stirred at room temperature for about one hour. After completion, the reaction mixture was concentrated under reduced pressure and was directly purified through a 60-120 mesh size silica gel column chromatograph using 15% ethyl acetate in hexane as eluent to obtain the title compound.

Yield: 330 mg

### Step 4: Synthesis of 2-{[2-(4′-chlorobiphenyl-4-yl)ethyl]sulfanyl}-4-(6-fluoro-4-oxo-1,2,3-benzotriazin-3(4H)-yl)butanoic acid

In a solution of 2-[2-(4′-chloro-biphenyl-4-yl)-ethylsulfanyl]-4-(6-fluoro-4-oxo-4H-benzo[d][1,2,3]triazin-3-yl)-butyric acid methyl ester (0.3 g, 0.821 mmol) in tetrahydrofuran:methanol:water (3 mL:1 mL:1 mL), lithium hydroxide (50 mg, 0.034 mol) was added at ˜0° C. This was stirred at room temperature for about 2 hours. The crude reaction mixture was diluted with ethyl acetate, acidified with sodium bisulphate and then extracted in ethyl acetate. Purification was done in 2 mm preparative TLC using 10% methanol in dichloromethane as eluent to obtain the title compound.

Yield: 122 mg

LCMS: 496.3 (M−1)

NMR (DMSO-d₆, 400 MHz): δ 7.9 (1H, d, J=8.4 Hz), 7.654 (2H, d, J=8.4 Hz), 7.560 (2H, d, J=8 Hz), 7.481 (2H, d, J=8.4 Hz), 7.303 (2H, d, J=8 Hz), 4.47 (2H, s), 2.86-2.66 (4H, m), 2.48-2.31 (2H, m).

The following compounds were prepared by following the above synthetic route:
2-{[2-(4′-Chlorobiphenyl-4-yl)ethyl]sulfanyl}-4-(7-chloro-4-oxo-1,2,3-benzotriazin-3(4H)-yl)butanoic acid (Compound no. 163)

Mass: 514.27 (M−1)
2-{[2-(4′-Chlorobiphenyl-4-yl)ethyl]sulfanyl}-4-(7-methyl-4-oxo-1,2,3-benzotriazin-3(4H)-yl)butanoic acid (Compound no. 164)

Mass: 494.44 (M−1)
2-{[2-(4′-Chlorobiphenyl-4-yl)ethyl]sulfanyl}-4-(8-methyl-4-oxo-1,2,3-benzotriazin-3(4H)-yl)butanoic acid (Compound no. 165)

Mass: 494.37 (M−1)
2-{[2-(4′-Chlorobiphenyl-4-yl)ethyl]sulfanyl}-4-(6-methyl-4-oxo-1,2,3-benzotriazin-3(4H)-yl)butanoic acid (Compound no. 196)

Mass: 494.29 (M−1)

### Synthetic procedure for Scheme II

### Example 4

### Synthesis of 4-(7-methoxy-4-oxo-1,2,3-benzotriazin-3(4H)-yl)-2-{[4′-(trifluoromethyl)biphenyl-4-yl]sulfonyl}butanoic acid (Compound no. 48)

To a solution of 4-(7-methoxy-4-oxo-1,2,3-benzotriazin-3(4H)-yl)-2-{[4′-(trifluoro methyl)biphenyl-4-yl]sulfanyl}butanoic acid (0.1 g, 0.00019 mol) in chloroform (10 mL), metachloroperbenzoic acid (0.133 g, 0.00077 mol) was added at about 0° C. This reaction mixture was stirred for about 1 hour. The resultant reaction mixture was extracted in dichloromethane, organic layer was dried with sodium sulphate and concentrated, purified by preperative TLC and eluted in 10% methanol/dichloromethane to obtain the title compound.

Yield: 0.030 g

LCMS: 548.09 (M+1)

NMR (DMSO-d₆, 400 MHz): δ 8.09-8.12 (2H, d, J=12 Hz), 7.84-7.99 (7H, m), 7.58-7.59 (1H, d, J=4 Hz), 7.40-7.43 (1H, d, J=4 Hz), 4.41-4.45 (2H, m), 3.95-3.98 (3H, s), 3.89 (1H, m), 2.36 (2H, m).

### Example 5

### Synthesis of 2-{[(4′-chlorobiphenyl-4-yl)methyl]sulfonyl}-4-(4-oxo-1,2,3-benzotriazin-3(4H)-yl)butanoic acid (Compound no. 6)

To a solution of 2-{[(4′-chlorobiphenyl-4-yl)methyl]sulfanyl}-4-(4-oxo-1,2,3-benzotriazin-3(4H)-yl)butanoic acid (0.02 g, 0.430 mmol) in methanol (2 mL), a solution of oxone (0.1 g) in minimum amount of water was added. The reaction mixture was stirred overnight and then extracted in ethyl acetate, washed with water and brine solution. The organic layer was dried over sodium sulphate and concentrated to obtain title product.

Yield: 40 mg

Mass: 510.24 (M−1)

NMR (DMSO-d₆, 400 MHz): δ 8.14 (1H, d, J=8 Hz), 7.88 (1H, s), 7.70-7.68 (2H, m), 7.64-7.56 (2H, m), 7.54-7.51 (5H, m), 4.78-4.64 (1H, m), 4.59-4.43 (2H, m), 4.31-4.21 (2H, m), 2.70-2.58 (2H, m), 2.54 (3H, s).

The following compounds were prepared by following any of the above synthetic routes:
2-[(3′-Methoxybiphenyl-4-yl)sulfonyl]-4-(4-oxo-1,2,3-benzotriazin-3(4H)-yl)butanoic acid (Compound no. 2)

Mass: 480.22
2-[(3′-Fluoro-4′-methoxybiphenyl-4-yl)sulfonyl]-4-(6-methyl-4-oxo-1,2,3-benzotriazin-3(4H)-yl)butanoic acid (Compound no. 3)

Mass: 512.19
2-{[2-(4′-Chlorobiphenyl-4-yl)ethyl]sulfonyl}-4-(4-oxo-1,2,3-benzotriazin-3(4H)-yl)butanoic acid (Compound no. 4)

Mass: 510.30
2-{[2-(4′-Chlorobiphenyl-4-yl)ethyl]sulfonyl}-4-(6-fluoro-4-oxo-1,2,3-benzotriazin-3(4H)-yl)butanoic acid (Compound no. 5)

Mass: 528.11 (M−1)
2-{[(4′-Chlorobiphenyl-4-yl)methyl]sulfonyl}-4-(4-oxo-1,2,3-benzotriazin-3(4H)-yl)butanoic acid (Compound no. 7)

Mass: 496.32 (M−1)
2-[(3′,4′-Difluorobiphenyl-4-yl)sulfonyl]-4-(6-methyl-4-oxo-1,2,3-benzotriazin-3(4H)-yl)butanoic acid (Compound no. 8)

Mass: 510.17 (M−1)
2-{[(4′-Chlorobiphenyl-4-yl)methyl]sulfonyl}-4-[4-oxo-7-(trifluoromethyl)-1,2,3-benzotriazin-3(4H)-yl]butanoic acid (Compound no. 9)

Mass: 564.29 (M−1)

4-(6-Methyl-4-oxo-1,2,3-benzotriazin-3(4H)-yl)-2-{[4′-(trifluoromethyl)biphenyl-4-yl]sulfonyl}butanoic acid (Compound no. 10)

Mass: 532.16
2-[(3′,4′-Dichlorobiphenyl-4-yl)sulfonyl]-4-(6-methyl-4-oxo-1,2,3-benzotriazin-3(4H)-yl)butanoic acid (Compound no. 11)

Mass: 533.90/532.09
2-[(4′-Methoxy-3′-methylbiphenyl-4-yl)sulfonyl]-4-(6-methyl-4-oxo-1,2,3-benzotriazin-3(4H)-yl)butanoic acid (Compound no. 12)

Mass: 508.22
2-[(3′,4′-Difluorobiphenyl-4-yl)sulfonyl]-4-(6-methyl-4-oxo-1,2,3-benzotriazin-3(4H)-yl)butanoic acid (Compound no. 13)

Mass: 500.15
2-[(3′-Fluoro-4′-methylbiphenyl-4-yl)sulfonyl]-4-(6-methyl-4-oxo-1,2,3-benzotriazin-3(4H)-yl)butanoic acid (Compound no. 14)

Mass: 496.19
2-[(4′-Fluorobiphenyl-4-yl)sulfonyl]-4-(6-methyl-4-oxo-1,2,3-benzotriazin-3(4H)-yl)butanoic acid (Compound no. 15)

Mass: 482.21
2-[(4′-Chlorobiphenyl-4-yl)sulfonyl]-4-(6-methyl-4-oxo-1,2,3-benzotriazin-3(4H)-yl)butanoic acid (Compound no. 16)

Mass: 498.12
2-[(4′-Ethylbiphenyl-4-yl)sulfonyl]-4-(6-methyl-4-oxo-1,2,3-benzotriazin-3(4H)-yl)butanoic acid (Compound no. 17)

Mass: 492.20
2-[(4′-Methoxybiphenyl-4-yl)sulfonyl]-4-(6-methyl-4-oxo-1,2,3-benzotriazin-3(4H)-yl)butanoic acid (Compound no. 18)

Mass: 494.24
2-[(4′-Fluoro-3′-methylbiphenyl-4-yl)sulfonyl]-4-(6-methyl-4-oxo-1,2,3-benzotriazin-3(4H)-yl)butanoic acid (Compound no. 19)

Mass: 496.21

2-{[4-(6-Methoxypyridin-3-yl)benzyl]sulfonyl}-4-(4-oxo-1,2,3-benzotriazin-3(4H)-yl)butanoic acid (Compound no. 20)

Mass: 493.7 (M−1)
2-{[2-(3′-Fluoro-4′-methoxybiphenyl-4-yl)ethyl]sulfonyl}-4-(4-oxo-1,2,3-benzotriazin-3(4H)-yl)butanoic acid (Compound no. 35)

Mass: 395.61 (M−130, fragmentation)
2-({2-[4-(1-Methyl-1H-pyrazol-4-yl)phenyl]ethyl}sulfonyl)-4-(4-oxo-1,2,3-benzotriazin-3(4H)-yl)butanoic acid (Compound no. 36)

Mass: 480.29
4-(7-Methyl-4-oxo-1,2,3-benzotriazin-3(4H)-yl)-2-{[4′-(trifluoromethoxy)biphenyl-4-yl]sulfonyl}butanoic acid (Compound no. 39)

Mass: 547.99
4-(6-Methoxy-4-oxo-1,2,3-benzotriazin-3(4H)-yl)-2-{[4′-(trifluoromethoxy)biphenyl-4-yl]sulfonyl}butanoic acid (Compound no. 40)

Mass: 563.97
4-(7-Methoxy-4-oxo-1,2,3-benzotriazin-3(4H)-yl)-2-{[4′-(trifluoromethoxy)biphenyl-4-yl]sulfonyl}butanoic acid (Compound no. 41)

Mass: 563.97
2-[(4′-tert-Butylbiphenyl-4-yl)sulfonyl]-4-(4-oxo-1,2,3-benzotriazin-3(4H)-yl)butanoic acid (Compound no. 42)

Mass: 506.12
2-[(4′-Ethylbiphenyl-4-yl)sulfonyl]-4-(4-oxo-1,2,3-benzotriazin-3(4H)-yl)butanoic acid (Compound no. 43)

Mass: 478.09
4-(4-Oxo-1,2,3-benzotriazin-3(4H)-yl)-2-{[4′-(propan-2-yl)biphenyl-4-yl]sulfonyl}butanoic acid (Compound no. 44)

Mass: 492.14
4-(4-Oxo-1,2,3-benzotriazin-3(4H)-yl)-2-{[4′-(trifluoromethoxy)biphenyl-4-yl]sulfonyl}butanoic acid (Compound no. 45)

Mass: 533.95
4-(7-Methoxy-4-oxo-1,2,3-benzotriazin-3(4H)-yl)-2-{[4′-(trifluoromethyl)biphenyl-4-yl]sulfonyl}butanoic acid (Compound no. 48)

Mass: 548.09
2-[(4′-Methoxybiphenyl-4-yl)sulfonyl]-4-(7-methoxy-4-oxo-1,2,3-benzotriazin-3(4H)-yl)butanoic acid (Compound no. 49)

Mass: 510.24

2-[(3′-Fluoro-4′-methylbiphenyl-4-yl)sulfonyl]-4-(7-methoxy-4-oxo-1,2,3-benzotriazin-3(4H)-yl)butanoic acid (Compound no. 50)

Mass: 512.21
2-[(3′,4′-Dimethoxybiphenyl-4-yl)sulfonyl]-4-(7-methoxy-4-oxo-1,2,3-benzotriazin-3(4H)-yl)butanoic acid (Compound no. 51)

Mass: 540.29
2-[(4′-Ethylbiphenyl-4-yl)sulfonyl]-4-(7-methoxy-4-oxo-1,2,3-benzotriazin-3(4H)-yl)butanoic acid (Compound no. 56)

Mass: 508.13
2-[(4′-Chlorobiphenyl-4-yl)sulfonyl]-4-(7-methoxy-4-oxo-1,2,3-benzotriazin-3(4H)-yl)butanoic acid (Compound no. 57)

Mass: 514.04
2-[(3′,4′-Dichlorobiphenyl-4-yl)sulfonyl]-4-(7-methoxy-4-oxo-1,2,3-benzotriazin-3(4H)-yl)butanoic acid (Compound no. 58)

Mass: 549.91
2-[(3′-Fluoro-4′-methoxybiphenyl-4-yl)sulfonyl]-4-(7-methoxy-4-oxo-1,2,3-benzotriazin-3(4H)-yl)butanoic acid (Compound no. 59)

Mass: 528.08
2-[(3′,4′-Difluorobiphenyl-4-yl)sulfonyl]-4-(6-methoxy-4-oxo-1,2,3-benzotriazin-3(4H)-yl)butanoic acid (Compound no. 60)

Mass: 516.06
2-[(3′-Fluoro-4′-methylbiphenyl-4-yl)sulfonyl]-4-(6-methoxy-4-oxo-1,2,3-benzotriazin-3(4H)-yl)butanoic acid (Compound no. 61)

Mass: 512.16
2-[(4′-Fluoro-3′-methylbiphenyl-4-yl)sulfonyl]-4-(6-methoxy-4-oxo-1,2,3-benzotriazin-3(4H)-yl)butanoic acid (Compound no. 62)

Mass: 512.16
2-[(3′,4′-Dichlorobiphenyl-4-yl)sulfony]-4-(6-methoxy-4-oxo-1,2,3-benzotriazin-3(4H)-yl)butanoic acid (Compound no. 63)

Mass: 548.10
2-[(3′-Fluoro-4′-methoxybiphenyl-4-yl)sulfonyl]-4-(6-methoxy-4-oxo-1,2,3-benzotriazin-3(4H)-yl)butanoic acid (Compound no. 64)

Mass: 528.21
2-{[4′-Chloro-3′-(trifluoromethyl)biphenyl-4-yl]sulfonyl}-4-(6-methoxy-4-oxo-1,2,3-benzotriazin-3(4H)-yl)butanoic acid (Compound no. 65)

Mass: 581.96
2-[(4′-Ethylbiphenyl-4-yl)sulfonyl]-4-(6-methoxy-4-oxo-1,2,3-benzotriazin-3(4H)-yl)butanoic acid (Compound no. 66)

Mass: 508.17
2-[(3′,4′-Dimethylbiphenyl-4-yl)sulfonyl]-4-(6-methoxy-4-oxo-1,2,3-benzotriazin-3(4H)-yl)butanoic acid (Compound no. 67)

Mass: 508.17
4-(6-Methoxy-4-oxo-1,2,3-benzotriazin-3(4H)-yl)-2-[(4′-methylbiphenyl-4-yl)sulfonyl]butanoic acid (Compound no. 68)

Mass: 494.14
2-[(4′-Chlorobiphenyl-4-yl)sulfonyl]-4-(6-methoxy-4-oxo-1,2,3-benzotriazin-3(4H)-yl)butanoic acid (Compound no. 69)

Mass: 514.09
4-(6-Methoxy-4-oxo-1,2,3-benzotriazin-3(4H)-yl)-2-{[4′-(trifluoromethyl)biphenyl-4-yl]sulfonyl}butanoic acid (Compound no. 70)

Mass: 548.13
2-[(4′-Methoxybiphenyl-4-yl)sulfonyl]-4-(6-methoxy-4-oxo-1,2,3-benzotriazin-3(4H)-yl)butanoic acid (Compound no. 71)

Mass: 510.23
2-[(4′-Fluorobiphenyl-4-yl)sulfonyl]-4-(8-methyl-4-oxo-1,2,3-benzotriazin-3(4H)-yl)butanoic acid (Compound no. 89)

Mass: 482.2
2-[(3′-Fluoro-4′-methylbiphenyl-4-yl)sulfonyl]-4-(8-methyl-4-oxo-1,2,3-benzotriazin-3(4H)-yl)butanoic acid (Compound no. 90)

Mass: 496.3
2-[(4′-Fluoro-3′-methylbiphenyl-4-yl)sulfonyl]-4-(8-methyl-4-oxo-1,2,3-benzotriazin-3(4H)-yl)butanoic acid (Compound no. 91)

Mass: 496.3
2-[(3′,4′-Dimethylbiphenyl-4-yl)sulfonyl]-4-(8-methyl-4-oxo-1,2,3-benzotriazin-3(4H)-yl)butanoic acid (Compound no. 93)

Mass: 492.45
2-[(4′-Ethylbiphenyl-4-yl)sulfonyl]-4-(8-methyl-4-oxo-1,2,3-benzotriazin-3(4H)-yl)butanoic acid (Compound no. 96)

Mass: 492.38
2-[(4′-Methylbiphenyl-4-yl)sulfonyl]-4-(8-methyl-4-oxo-1,2,3-benzotriazin-3(4H)-yl)butanoic acid (Compound no. 97)

Mass: 478.46

2-{[4-(6-Methoxypyridin-3-yl)phenyl]sulfonyl}-4-(8-methyl-4-oxo-1,2,3-benzotriazin-3(4H)-yl)butanoic acid (Compound no. 98)

Mass: 495.43
2-[(4′-Methoxybiphenyl-4-yl)sulfanyl]-4-(7-methoxy-4-oxo-1,2,3-benzotriazin-3(4H)-yl)butanoic acid (Compound no. 197)

Mass: 524.15 (M−1)
4-(7-Methoxy-4-oxo-1,2,3-benzotriazin-3(4H)-yl)-2-{[4-(1-methyl-1H-pyrazol-4-yl)phenyl]sulfanyl}butanoic acid (Compound no. 199)

Mass: 525.89 (M−1)

### Synthetic Procedure for Scheme III

### Example 6 (when Rₘ is Br)

### Synthesis of 2-(4-Bromo-phenylmethanesulfonyl)-4-(4-oxo-4H-benzo[d][1,2,3]triazin-3-yl)-butvric acid ethyl ester (Intermediate of Compound No. 1)

### Step 1: Synthesis of ethyl[(4-bromobenzyl)sulfonyl]acetate

To a solution of ethyl[(4-bromobenzyl)sulfanyl]acetate (5 g, 0.0173 mol) in dichloromethane, m-chloroperbenzoic acid (8.95 g, 0.0519 mol) was added at about 0° C. and was stirred at room temperature for about 2 hours. The reaction mixture was then extracted in dichloromethane-water and the organic layer was washed with saturated sodium bicarbonate solution. The crude compound was then purified in a 60-120 mesh size silica gel column chromatograph and pure title compound was obtained in 50% ethyl acetate hexane.

Yield: 4.2 g

MS: 321.24 (M−1)

### Step 2: Synthesis of 2-(4-bromo-phenylmethanesulfonyl)-4-(4-oxo-4H-benzo[d][1,2,3]triazin-3-yl)-butyric acid ethyl ester

(4-Bromo-phenylmethanesulfonyl)-acetic acid ethyl ester (0.250 g, 0.778 mmol), benzotriazinone ethylbromide (0.237 g, 0.934 mol), potassium carbonate (0.322 g, 2.33 mmol) and tetrabutyl ammonium iodide (72 mg, 0.194 mol) were taken together in N,N′-dimethylformamide (5 mL) and heated overnight at about 80° C. The crude reaction mixture was then extracted in ethyl acetate and washed with water and brine solution. It was purified in 60-120 mesh size silica gel column chromatograph to obtain the pure title compound in 20% ethyl acetate hexane.

Yield: 214 mg

MS: 494.24 (M−1)

### Synthetic procedure for Scheme III

### Example 7 (when Rₘ is Br)

### Synthesis of propyl 2-[(4-nitrophenyl)sulfonyl]-4-(4-oxo-1,2,3-benzotriazin-3(4H)-yl)butanoate (Intermediate of Compound no. 1)

### Step 1: Synthesis of ethyl[(4-nitrophenyl)sulfonyl]acetate

To a solution of ethyl[(4-nitrophenyl)sulfanyl]acetate (6.5 g, 0.0269 mol) in chloroform (70 mL) at about 0° C., meta chloroperbenzoic acid (18 g, 0.107 mol) was added and this reaction mixture was stirred for about 1 hour. The reaction was quenched by sodium meta bisulphite solution. The crude title compound was extracted in dichloromethane, dried with sodium sulphate and concentrated, purified by preperative TLC eluted in 10% ethanol/dichloromethane.

Yield: 6.7 g

LCMS: 274 (M+1)

### Step 2: Synthesis of ethyl 2-[(4-nitrophenyl)sulfonyl]-4-(4-oxo-1,2,3-benzotriazin-3(4H)-yl)butanoate

To a solution of ethyl[(4-nitrophenyl)sulfonyl]acetate (0.5 g, 0.0018 mol) in dimethylformamide (5 mL) under argon atmosphere, potassium carbonate (0.745 g, 0.0054 mol), tetrabutylammonium iodide (TBAI) (0.066 g, 0.0001 mol) and benzotriazinone ethyl bromide (0.697 g, 0.0027 mol) were added. The reaction mixture was stirred for about 4 hours at about 50° C. The crude compound was extracted in ethyl acetate, dried the organic layer with sodium sulphate and concentrated and purified by silica gel column with 8% ethyl acetate/hexane to obtain the title compound.

Yield: 0.65 g

LCMS: 447.36 (M+1)

### Synthetic Procedure for Scheme IV

### Example 8

### Synthesis of ethyl 2-[(4-aminophenyl)sulfonyl]-4-(4-oxo-1,2,3-benzotriazin-3(4H)-yl)butanoate (Intermediate of Compound no. 53)

### Step 1: Synthesis of methyl[(4-aminophenyl)sulfanyl]acetate

To a solution of 4-aminothioglycolic acid (34 g) in methanol (200 mL), sulfuric acid (10 mL) was added at room temperature, then reaction mixture was heated to −70° C. for about 12 hours.

Solvent was evaporated and reaction mixture was extracted in ethyl acetate while washing with water. Organic layer was collected, dried over anhydrous sodium sulphate. Then solvent was evaporated. Purification was done on silica gel column chromatography using 20% ethyl acetate:hexane as eluent to get the desired title product.

Yield: 37 g

LCMS (m/z): 198 (M+1)

### Step 2: Synthesis of methyl({4-[(tert-butoxycarbonyl)amino]phenyl}sulfanyl)acetate

To a solution of methyl[(4-aminophenyl)sulfanyl]acetate (31 g, 0.157 mol) in dichloromethane (250 mL) at room temperature, di-tert-butyl dicarbonate (100 mL, 0.472 mol) and triethylamine (26 mL, 0.188 mol) were added. Then the reaction mixture was stirred for about 6 hours at room temperature. After that, the reaction mixture was extracted in dichloromethane while washing with water. Organic layer was collected, dried over anhydrous sodium sulphate. Then the solvent was evaporated under reduced pressure and purification was done on silica gel column chromatography using 30% ethyl acetate:hexane as eluent to get the desired title product.

Yield: 56 g

LCMS (m/z): 298.22 (M+1)

### Step 3: Synthesis of methyl({4-[(tert-butoxycarbonyl)amino]phenyl}sulfonyl)acetate

To a solution of methyl({4-[(tert-butoxycarbonyl)amino]phenyl}sulfanyl)acetate (56 g, 0.170 mol) in chloroform (400 mL), m-chloroperbenzoic acid (88 g, 0.150 mol) was added at room temperature then reaction mixture was stirred for about 3 hours at the same temperature. After that, aqueous solution of sodium bicarbonate was added to the reaction mixture then extracted with dichloromethane. Organic layer was dried over anhydrous sodium sulphate. Then solvent was evaporated to get the desired title product.

Yield: 42 g

LCMS (m/z): 347.47 (M+NH₄⁺)

### Step 4: Synthesis of methyl 2-({4-[(tert-butoxycarbonyl)amino]phenyl}sulfonyl)-4-(4-oxo-1,2,3-benzotriazin-3(4H)-yl)butanoate

To a solution of methyl({4-[(tert-butoxycarbonyl)amino]phenyl}sulfonyl)acetate (11 g, 0.033 mol) in N,N′-dimethylformamide (150 mL) at room temperature, benzotriazinone ethyl bromide (12.7 g, 0.050 mol), tetrabutylammonium iodide (nBu₄NI) (3.08 g, 0.008 mol) and potassium carbonate (13.8 g, 0.100 mol) were added and then the reaction mixture was heated up to 50° C. for about 6 hours. After that, solvent was evaporated and reaction mixture was extracted in ethyl acetate while washing with water, organic layer was collected and dried over anhydrous sodium sulphate. Solvent was evaporated and purification was done on silica gel (60-120 mesh) column using 40% ethyl acetate:hexane as eluent to get the desired title product.

Yield: 11 g

LCMS (m/z): 503.40 (M+1)

### Step 5: Synthesis of methyl 2-[(4-aminophenyl)sulfonyl]-4-(4-oxo-1,2,3-benzotriazin-3(4H)-yl)butanoate

To a solution of methyl 2-({4-[(tert-butoxycarbonyl)amino]phenyl}sulfonyl)-4-(4-oxo-1,2,3-benzotriazin-3(4H)-yl)butanoate (11 g) in dichloromethane (100 mL) at about 0° C., trifluoroacetic acid (22 mL) was added and the reaction mixture was stirred at room temperature for about 3 hours. Subsequently, the reaction mixture was concentrated and aqueous solution of sodium bicarbonate was added and extracted with ethyl acetate while washing with water. Organic layer was collected and dried over anhydrous sodium sulphate. Solvent was evaporated to get the desired product.

Yield: 9 g

LCMS (m/z): 403.12 (M+1)

### Synthetic Procedure for Scheme V

### Path C (when Rₘ is Br)

### Example 9

### Synthesis of 4-(4-oxo-1,2,3-benzotriazin-3(4H)-yvl)-2-({[4′-(trifluoromethoxy)biphenyl-4-yl]methyl}sulfonyl)butanoic acid (Compound no. 21)

### Step 1: Synthesis of methyl 4-(4-oxo-1,2,3-benzotriazin-3(4H)-yl)-2-({[4′-(trifluoro-methoxy)biphenyl-4-yl]methyl}sulfonyl)butanoate

To a solution of 2-(4-bromo-phenylmethanesulfonyl)-4-(4-oxo-4H-benzo[d][1,2,3]triazin-3-yl)-butyric acid ethyl ester (0.25 g, 0.506 mmol) in N,N′-dimethylformamide (5 mL), potassium carbonate (0.209 g, 1.51 mmol) was added. To this mixture, trifluoromethoxy phenylboronic acid (0.166 g, 0.809 mmol) and tetrakis triphenylphosphine palladium (0) (58.4 g, 0.05 mol) were added. The resulting solution was heated at about 110° C. for duration of about 6 hours. Further, the reaction mixture was cooled to room temperature, added water and extracted twice in ethyl acetate. The organic layer was separated and dried under sodium sulphate and concentrated in reduced pressure. Purification of the crude compound was done on silica gel column chromatograph using 20% ethyl acetate-hexane as eluent to obtain the title compound.

Yield: 214 mg

MS −521.40 (M−1)

### Step 2: Synthesis of 4-(4-oxo-1,2,3-benzotriazin-3(4H)-yl)-2-({[4′-(trifluoromethoxy)biphenyl-4-yl]methyl}sulfonyl)butanoic acid

To a solution of methyl 4-(4-oxo-1,2,3-benzotriazin-3(4H)-yl)-2-({[4′-(trifluoro-methoxy)biphenyl-4-yl]methyl}sulfonyl)butanoate (0.141 g, 0.000245 mol) in tetrahydrofuran:methanol:water (3 mL:1 mL:1 mL), lithium hydroxide (11 mg, 0.000262 mol) was added at about 0° C. The reaction mixture was stirred at room temperature for about 2 hours. The crude reaction mixture was first diluted with ethyl acetate, acidified with sodium bisulphate and then extracted in ethyl acetate. Purification was done 2 mm preparative TLC using 10% methanol in dichloromethane as eluent to obtain the title compound.

Yield: 112 mg

Mass: 546.39 (M−1)

NMR (DMSO-d₆+D₂O, 400 MHz): δ 8.243-7.868 (4H, m), 7.757 (2H, d, J=8.4 Hz), 7.605 (2H, d, J=8.4 Hz), 7.497-7.296 (4H, m), 4.912-4.878 (1H, m), 4.583-4.480 (3H, m), 3.686-3.667 (1H, m), 2.439-2.360 (2H, m).

The following compounds were prepared by following above synthetic route:
2-{[(3′,4′-Dimethoxybiphenyl-4-yl)methyl]sulfonyl}-4-(4-oxo-1,2,3-benzotriazin-3(4H)-yl)butanoic acid (Compound no. 22)

Mass: 522.68
2-{[(3′-Fluoro-4′-methylbiphenyl-4-yl)methyl]sulfonyl}-4-(4-oxo-1,2,3-benzotriazin-3(4H)-yl)butanoic acid (Compound no. 23)

Mass: 494.44
2-{[(3′,4′-Dimethylbiphenyl-4-yl)methyl]sulfonyl}-4-(4-oxo-1,2,3-benzotriazin-3(4H)-yl)butanoic acid (Compound no. 24)

Mass: 490.34
2-{[(3′,4′-Dichlorobiphenyl-4-yl)methyl]sulfonyl}-4-(4-oxo-1,2,3-benzotriazin-3(4H)-yl)butanoic acid (Compound no. 25)

Mass: 535.18 (M−1+4) Di-chloro pattern
2-{[(4′-Fluoro-3′-methylbiphenyl-4-yl)methyl]sulfonyl}-4-(4-oxo-1,2,3-benzotriazin-3(4H)-yl)butanoic acid (Compound no. 26)

Mass: 494.44
4-(4-Oxo-1,2,3-benzotriazin-3(4H)-yl)-2-({[4′-(trifluoromethyl)biphenyl-4-yl]methyl}sulfonyl)butanoic acid (Compound no. 27)

Mass: 530.34
2-{[(4′-Methoxybiphenyl-4-yl)methyl]sulfonyl}-4-(4-oxo-1,2,3-benzotriazin-3(4H)-yl)butanoic acid (Compound no. 28)

Mass: 492.35
2-{[(4′-Fluorobiphenyl-4-yl)methyl]sulfonyl}-4-(4-oxo-1,2,3-benzotriazin-3(4H)-yl)butanoic acid (Compound no. 29)

Mass: 480.22

2-{[2-(3′-Fluoro-4′-methylbiphenyl-4-yl)ethyl]sulfonyl}-4-(4-oxo-1,2,3-benzotriazin-3(4H)-yl)butanoic acid (Compound no. 31)

Mass: 507.94
2-{[2-(4′-Ethylbiphenyl-4-yl)ethyl]sulfonyl}-4-(4-oxo-1,2,3-benzotriazin-3(4H)-yl)butanoic acid (Compound no. 32)

Mass: 504.28
2-{[2-(3′,4′-Difluorobiphenyl-4-yl)ethyl]sulfonyl}-4-(4-oxo-1,2,3-benzotriazin-3(4H)-yl)butanoic acid (Compound no. 33)

Mass: 512.41
2-{[2-(4′-Cyanobiphenyl-4-yl)ethyl]sulfonyl}-4-(4-oxo-1,2,3-benzotriazin-3(4H)-yl)butanoic acid (Compound no. 34)

Mass: 501.21
2-{[2-(4′-Methylbiphenyl-4-yl)ethyl]sulfonyl}-4-(4-oxo-1,2,3-benzotriazin-3(4H)-yl)butanoic acid (Compound no. 37)

Mass: 492.35
4-(4-Oxo-1,2,3-benzotriazin-3(4H)-yl)-2-({2-[4′-(trifluoromethoxy)biphenyl-4-yl]ethyl}sulfonyl)butanoic acid (Compound no. 38)

Mass: 560.58

### Path D: (when Rₘ is NO₂)

### Example 10

### Synthesis of methyl 2-[(4-aminophenyl)sulfonyl]-4-(4-oxo-1,2,3-benzotriazin-3(4H)-yl)butanoate

To the solution of methyl 2-[(4-nitrophenyl)sulfonyl]-4-(4-oxo-1,2,3-benzotriazin-3(4H)-yl)butanoate (0.650 g, 0.00014 mol) in tetrahydrofuran (100 mL)/methanol (100 mL), Pd/C (0.26 g) was added and then vacuum followed by hydrogen pressure by balloon was applied, stirred for about 5 hours. After completion, the reaction mixture was filtered and concentrated to obtain the title compound.

Yield: 0.6 g

LCMS (m/z): 417.37 (M+1)

### Path E

### Example 11

### Synthesis of 2-[(4-{[(4-fluorophenyl)carbamoyl]amino}phenyl)sulfonyl]-4-(4-oxo-1,2,3-benzotriazin-3(4H)-yl)butanoic acid (Compound no. 88)

### Step 1: Synthesis of methyl 2-[(4-{[(4-fluorophenyl)carbamoyl]amino}phenyl)sulfonyl]-4-(4-oxo-1,2,3-benzotriazin-3(4H)-yl)butanoate

To a solution of methyl 2-[(4-aminophenyl)sulfonyl]-4-(4-oxo-1,2,3-benzotriazin-3(4H)-yl)butanoate (0.42 g, 1.044 mmol) in tetrahydrofuran (15 mL) at 0° C., triethyl amine (0.16 g, 1.567 mmol) and 4-fluorophenyl isocyanate (0.16 g, 1.149 mmol) were added. The reaction mixture was stirred for about 1 hour at room temperature. The resultant mixture was extracted in ethyl acetate. The organic layer was collected, dried over anhydrous sodium sulphate. Purification was performed on silica gel (60-120 mesh) column using 60% ethyl acetate:hexane as eluent to get the desired product.

Yield: 300 mg

LCMS (m/z): 540.1 (M+1)

### Step 2: Synthesis of 2-[(4-{[(4-fluorophenyl)carbamoyl]amino}phenyl)sulfonyl]-4-(4-oxo-1,2,3-benzotriazin-3(4H)-yl)butanoic acid

To a solution of methyl 2-[(4-{[(4-fluorophenyl)carbamoyl]amino}phenyl)sulfonyl]-4-(4-oxo-1,2,3-benzotriazin-3(4H)-yl)butanoate (0.3 g, 0.556 mmol) in tetrahydrofuran:methanol:water (8 mL each), lithium hydroxide (0.035 g, 0.834 mmol) was added. The reaction mixture was stirred for about 2 hours at the room temperature.

After completion of reaction, the reaction mixture was concentrated, acidified using aqueous solution of sodium bisulphate and extracted with ethyl acetate. Organic layer was dried over anhydrous sodium sulphate and the solvent was evaporated. Purification was done by preparative TLC plates (thickness 2 mm) while using 20% methanol:dichloromethane to get the desired product.

Yield: 120 mg

LCMS (m/z): 526.33 (M+1)

¹HNMR (DMSO-d₆, 400 MHz); δ 11.06 (1H, s, COOH), 9.99 (1H, s, NH), 8.21 (2H, m), 8.15 (2H, m), 7.90 (4H, m), 7.03 (4H, m), 4.4 (2H, m), 3.78 (1H, m), 2.30 (2H, m)

The following compounds were prepared by following above synthetic route
2-[(4-{[(3-Ethoxyphenyl)carbamoyl]amino}phenyl)sulfonyl]-4-(4-oxo-1,2,3-benzotriazin-3(4H)-yl)butanoic acid (Compound no. 85)

Mass: 552.50 (M+1), 574.46 (M+Na)
2-{[4-({[2-Fluoro-5-(trifluoromethyl)phenyl]carbamoyl}amino)phenyl]sulfonyl}-4-(4-oxo-1,2,3-benzotriazin-3(4H)-yl)butanoic acid (Compound no. 86)

Mass: 616.70 (M+Na)

### Path F

### Example 12

### Synthesis of 2-[(4-{[(3-fluorophenyl)carbonyl]amino}phenyl)sulfonyl]-4-(4-oxo-1,2,3-benzotriazin-3(4H)-yl)butanoic acid (Compound no. 53)

### Step 1: Synthesis of methyl 2-[(4-{[(3-fluorophenyl)carbonyl]amino}phenyl)sulfonyl]-4-(4-oxo-1,2,3-benzotriazin-3(4H)-yl)butanoate

To a solution of methyl 2-[(4-aminophenyl)sulfonyl]-4-(4-oxo-1,2,3-benzotriazin-3(4H)-yl)butanoate (0.2 g, 0.497 mmol) in dichloromethane at 0° C., triethylamine (0.2 g, 1.990 mmol) and 3-fluorobenzoyl chloride (0.23 g, 1.492 mmol) were added. The reaction mixture was stirred at room temperature for about 2 hours and then extracted with dichloromethane while washing with water. The organic layer was collected, dried over anhydrous sodium sulphate. Solvent was evaporated and purification was performed over silica gel column using 30% ethyl acetate:hexane as eluent to get the title product.

Yield: 350 mg

LCMS (m/z): 525.22 (M+1)

### Step 2: Synthesis of 2-[(4-{[(3-fluorophenyl)carbonyl]amino}phenyl)sulfonyl]-4-(4-oxo-1,2,3-benzotriazin-3(4H)-yl)butanoic acid

To a solution of methyl 2-[(4-{[(3-fluorophenyl)carbonyl]amino}phenyl)sulfonyl]-4-(4-oxo-1,2,3-benzotriazin-3(4H)-yl)butanoate (0.35 g, 0.667 mmol) in tetrahydrofuran:methanol:water (10 mL: 10 mL:5 mL), lithium hydroxide (0.042 g, 1.001 mmol) was added at room temperature. The reaction mixture was stirred for about 2 hours. After that, the mixture was concentrated, acidified using aqueous solution of sodium bisulphate and then extracted with ethyl acetate. The organic layer was collected, dried over anhydrous sodium sulphate and solvent was evaporated. Purification was performed by preparative TLC plates (thickness 2 mm) while using 20% MeOH:CH₂Cl₂ as mobile phase to get the desired product.

Yield: 20 mg

MS (m/z): 511.18 (M+1)

¹HNMR (DMSO-d₆, 400 MHz); δ 10.7 (1H, s, COOH), 8.21 (2H, m), 8.17 (2H, m), 7.86 (6H, m), 7.61 (1H, m), 7.11 (1H, m), 4.43 (2H, m), 3.79 (1H, m), 2.29 (2H, m)

The following compounds were prepared by following above synthetic route:
2-[(4-{[(4-Methylphenyl)carbonyl]amino}phenyl)sulfonyl]-4-(4-oxo-1,2,3-benzotriazin-3(4H)-yl)butanoic acid (Compound no. 1)

Mass: 507.30 (M+1)
4-(4-Oxo-1,2,3-benzotriazin-3 (4H-yl)-2-({4-[(phenylcarbonyl)amino]phenyl}sulfonyl)butanoic acid (Compound no. 46)

Mass: 493.15 (M+1)
2-[(4-{[(4-Methoxyphenyl)carbonyl]amino}phenyl)sulfonyl]-4-(4-oxo-1,2,3-benzotriazin-3(4H)-yl)butanoic acid (Compound no. 47)

Mass: 523.20 (M+1)
2-[(4-{[(3-Methoxyphenyl)carbonyl]amino}phenyl)sulfonyl]-4-(4-oxo-1,2,3-benzotriazin-3(4H)-yl)butanoic acid (Compound no. 52)

Mass: 523.22 (M+1)
2-[(4-{[(3-Fluorophenyl)carbonyl]amino}phenyl)sulfonyl]-4-(4-oxo-1,2,3-benzotriazin-3(4H)-yl)butanoic acid (Compound no. 53)

Mass: 511.18
2-[(4-{[(4-Fluorophenyl)carbonyl]amino}phenyl)sulfonyl]-4-(4-oxo-1,2,3-benzotriazin-3(4H)-yl)butanoic acid (Compound no. 54)

Mass: 511.37 (M+1)
2-[(4-{[(4-Chlorophenyl)carbonyl]amino}phenyl)sulfonyl]-4-(4-oxo-1,2,3-benzotriazin-3(4H)-yl)butanoic acid (Compound no. 55)

Mass: 527.25 (M+1)
4-(4-Oxo-1,2,3-benzotriazin-3(4H)-yl)-2-({2-[4-({[4-(trifluoromethyl)phenyl]carbonyl}amino phenyl]ethyl}sulfonyl)butanoic acid (Compound no. 72)

Mass: 587.22
4-(4-Oxo-1,2,3-benzotriazin-3(4H)-yl)-2-({2-[4-({[4-(trifluoromethoxy)phenyl]carbonyl}amino)phenyl]ethyl}sulfonyl)butanoic acid (Compound no. 73)

Mass: 603.21
2-[(4-{[(3,4-Dichlorophenyl)carbonyl]amino}phenyl)sulfonyl]-4-(4-oxo-1,2,3-benzotriazin-3(4H)-yl)butanoic acid (Compound no. 74)

Mass: 561.09 (M+1)
4-(4-Oxo-1,2,3-benzotriazin-3(4H)-yl)-2-[(2-{4-[(phenylcarbonyl)amino]phenyl}ethyl)sulfonyl]butanoic acid (Compound no. 76)

Mass: 519.31
4-(4-Oxo-1,2,3-benzotriazin-3(4H)-yl)-2-[(2-{4-[(thiophen-2-ylcarbonyl)amino]phenyl}ethyl)sulfonyl]butanoic acid (Compound no. 77)

Mass: 525.22
2-[(2-{4-[(Cyclopentylcarbonyl)amino]phenyl}ethyl)sulfonyl]-4-(4-oxo-1,2,3-benzotriazin-3(4H)-yl)butanoic acid (Compound no. 78)

Mass: 511.40
2-[(2-{4-[(Cyclopropylcarbonyl)amino]phenyl}ethyl)sulfonyl]-4-(4-oxo-1,2,3-benzotriazin-3(4H)-yl)butanoic acid (Compound no. 79)

Mass: 483.42
2-{[2-(4-{[(3-Methoxyphenyl)carbonyl]amino}phenyl)ethyl]sulfonyl}-4-(4-oxo-1,2,3-benzotriazin-3(4H)-yl)butanoic acid (Compound no. 80)

Mass: 549.30
2-{[2-(4-{[(3-Chlorophenyl)carbonyl]amino}phenyl)ethyl]sulfonyl}-4-(4-oxo-1,2,3-benzotriazin-3(4H)-yl)butanoic acid (Compound no. 81)

Mass: 553.32
2-{[2-(4-{[(3-Fluorophenyl)carbonyl]amino}phenyl)ethyl]sulfonyl}-4-(4-oxo-1,2,3-benzotriazin-3(4H)-yl)butanoic acid (Compound no. 82)

Mass: 537.36
2-{[2-(4-{[(4-Ethylphenyl)carbonyl]amino}phenyl)ethyl]sulfonyl}-4-(4-oxo-1,2,3-benzotriazin-3(4H)-yl)butanoic acid (Compound no. 83)

Mass: 546.95
2-[(4-{[(2,6-Dimethoxyphenyl)carbonyl]amino}phenyl)sulfonyl]-4-(4-oxo-1,2,3-benzotriazin-3(4H)-yl)butanoic acid (Compound no. 87)

Mass: 553.41 (M+1)
2-({4-[(Cyclohexylcarbonyl)amino]phenyl}sulfonyl)-4-(4-oxo-1,2,3-benzotriazin-3(4H)-yl)butanoic acid (Compound no. 99)

Mass: 499.38 (M+1), 521.41 (M+Na)
2-[(4-{[(2-Methylphenyl)carbonyl]amino}phenyl)sulfonyl]-4-(4-oxo-1,2,3-benzotriazin-3(4H)-yl)butanoic acid (Compound no. 100)

Mass: 507.35 (M+1), 529.37 (M+Na)
2-({4-[(Cyclopropylcarbonyl)amino]phenyl}sulfonyl)-4-(4-oxo-1,2,3-benzotriazin-3(4H)-yl)butanoic acid (Compound no. 101)

Mass: 457.40 (M+1), 479.36 (M+Na)
4-(4-Oxo-1,2,3-benzotriazin-3(4H)-yl)-2-({4-[(thiophen-2-ylcarbonyl)amino]phenyl}sulfonyl)butanoic acid (Compound no. 102)

Mass: 499.38 (M+1), 521.34 (M+1)
2-({4-[(Cyclopentylcarbonyl)amino]phenyl}sulfonyl)-4-(4-oxo-1,2,3-benzotriazin-3(4H)-yl)butanoic acid (Compound no. 103)

Mass: 485.39 (M+1), 507.41 (M+Na)
2-{[4-({[4-Fluoro-3-(trifluoromethyl)phenyl]carbonyl}amino)phenyl]sulfonyl}-4-(4-oxo-1,2,3-benzotriazin-3(4H)-yl)butanoic acid (Compound no. 104)

Mass: 579.32 (M+1), 601.21 (M+Na)
2-[(4-{[(3-Methoxyphenyl)acetyl]amino}phenyl)sulfonyl]-4-(4-oxo-1,2,3-benzotriazin-3(4H)-yl)butanoic acid (Compound no. 183)

Mass: 536.99 (M+1), 558.96 (M+Na)
2-[(4-{[(2,5-Dimethoxyphenyl)acetyl]amino}phenyl)sulfonyl]-4-(4-oxo-1,2,3-benzotriazin-3(4H)-yl)butanoic acid (Compound no. 184)

Mass: 566.96 (M+1), 588.93 (M+Na)
4-(4-Oxo-1,2,3-benzotriazin-3(4H)-yl)-2-({4-(phenylacetyl)amino]phenyl}sulfonyl)butanoic acid (Compound no. 185)

Mass: 528.93 (M+1)

### Synthetic Procedure for Scheme VI

### Example 13

### Synthesis of 4-(6-methoxy-4-oxo-1,2,3-benzotriazin-3(4H)-yl)-2-[(4-{[(4-methylphenyl)carbonyl]amino}phenyl)sulfanyl]butanoic acid (Compound no. 187)

### Path G

### Step 1: Synthesis of 3-[(4-aminophenyl)sulfanyl]dihydrofuran-2(3H)-one

To the solution of 3-[(4-nitrophenyl)sulfanyl]dihydrofuran-2(3H)-one (10.0 g, 0.04184 mol) in tetrahydrofuran (100 mL)/methanol (100 mL), Pd/C (4 g) was added and vacuum and then hydrogen pressure by balloon was applied. The reaction mixture was stirred for about 2 hours, and then filtered through celite and concentrated to obtain the title compound.

Yield: 5.0 g

Mass: 209

LCMS: 210.27 (M+1)

### Step 2: Synthesis of 4-methyl-N-{4-[(2-oxotetrahydrofuran-3-yl)sulfanyl]phenyl}benzamide

To a solution of 3-[(4-aminophenyl)sulfanyl]dihydrofuran-2(3H)-one (0.2 g, 0.0009 mol) in dichloromethane (10 mL), triethylamine (0.272 g, 0.0026 mol) was added under argon atmosphere at about 0° C. Afterwards, 4-methylbenzoyl chloride (0.162 g, 0.0010 mol) was added slowly drop wise for 15 minutes. The resultant reaction mixture was extracted in dichloromethane and washed with sodium bicarbonate. The organic layer was dried with sodium sulphate and concentrated, purified on silica gel column using 8% ethyl acetate/hexane as eluent to obtain the title compound.

Yield: 0.15 g

LCMS: 328.38 (M+1)

### Step 3: Synthesis of methyl 4-hydroxy-2-[(4-{[(4-methylphenyl)carbonyl]amino}phenyl)sulfanyl]butanoate

To a solution of 4-methyl-N-{4-[(2-oxotetrahydrofuran-3-yl)sulfanyl]phenyl}benzamide (0.3 g, 0.0008 mol) in dimethylformamide (4 mL) and water (1 mL), sodium hydroxide (0.040 g, 0.0010 mol) was added. The reaction mixture was stirred for about 30 minutes and then sodium bicarbonate (0.083 g, 0.0009 mol), 18 crown 6 (0.021 g, 0.00008 mol) and methyl iodide (0.177 g, 0.0012 mol) were added and stirred overnight. The reaction mixture was extracted in ethyl acetate, dried the organic layer with sodium sulphate and concentrated, purified by silica gel column using 8% ethyl acetate/hexane as eluent to obtain the title compound.

Yield: 0.3 g

### Step 4: Synthesis of methyl 4-(6-methoxy-4-oxo-1,2,3-benzotriazin-3(4H)-yl)-2-[(4-{[(4-methylphenyl)carbonyl]amino}phenyl)sulfanyl]butanoate

To a solution of the methyl 4-hydroxy-2-[(4-{[(4-methylphenyl)carbonyl]amino}phenyl)sulfanyl]butanoate (0.15 g, 0.00038 mol) in tetrahydrofuran (10 mL), 6-methoxybenzotrizinone (0.074 g, 0.00042 mol) and triphenyl phosphine (0.149 g, 0.00057 mol) were added under argon atmosphere. The reaction mixture was then cooled to 0° C. and DIAD (0.115 g, 0.00057 mol) was added to it and stirred for about 30 minutes. The mixture was extracted in ethyl acetate, dried the organic layer with sodium sulphate and concentrated, purified by silica gel column using 7% ethyl acetate/hexane as eluent to obtain the title compound.

Yield: 0.14 g

### Step 5: Synthesis of 4-(6-methoxy-4-oxo-1,2,3-benzotriazin-3(4H)-yl)-2-[(4-{[(4-methylphenyl)carbonyl]amino}) phenyl)sulfanyl]butanoic acid

To a solution of methyl 2-[(4-{[(4-methylphenyl)carbonyl]amino}phenyl) sulfanyl]-4-(4-oxo-1,2,3-benzotriazin-3(4H)-yl)butanoate (0.200 g, 0.0004 mol) in tetrahydrofuran (5 mL)/methanol (5 mL), a solution of lithium hydroxide (0.025 g, 0.0006 mol) in water was added and the reaction mixture was stirred for about 1 hour. The resultant mixture was acidified with sodium bisulphite solution and then extracted in ethyl acetate, dried the organic layer with sodium sulphate and concentrated, purified by preperative TLC using 10% methanol/dichloromethane as eluent to obtain the title compound.

Yield: 0.04 g

LCMS: 503.15 (M−1)

NMR (DMSO-d₆, 400 MHz): δ 10.20 (1H, s), 8.10-8.12 (1H, d, J=8 Hz), 7.84-7.86 (2H, d, J=8 Hz), 7.69-7.71 (2H, d, J=8 Hz), 7.54-7.60 (2H, d, J=8 Hz), 7.31-7.39 (4H, d, J=8 Hz), 4.49 (2H, m), 3.94 (3H, s), 3.71 (1H, m), 2.37 (1H, m), 2.15 (1H, m).

### Path H

### Example 14

### Synthesis of 4-(6-methyl-4-oxo-1,2,3-benzotriazin-3(4H)-yl)-2-[(4-{[(4-methylphenyl)carbonyl]amino}phenyl)sulfanyl]butanoic acid (Compound no. 202)

### Step 1: Synthesis of methyl 4-hydroxy-2-[(4-nitrophenyl)sulfanyl]butanoate

To a solution of 3-[(4-nitrophenyl)sulfanyl]dihydrofuran-2(3H)-one (2.0 g, 0.0083 mol) in dimethylformamide (8 mL) and water (2 mL), sodium hydroxide (0.4 g, 0.010 mol) was added and the reaction mixture was stirred for about 30 minutes. To it, sodium bicarbonate (0.836 g, 0.009 mol), 18 crown 6 (0.211 g, 0.0008 mol) and methyl iodide (1.7 g, 0.012 mol) were added and stirred overnight. The resultant mixture was extracted in ethyl acetate, dried the organic layer with sodium sulphate and concentrated, purified by silica gel column using 8% ethyl acetate/hexane as eluent to obtain the title compound.

Yield: 1.8 g

### Step 2: Synthesis of methyl 4-(6-methyl-4-oxo-1,2,3-benzotriazin-3(4H)-yl)-2-[(4-nitrophenyl)sulfanyl]butanoate

To a solution of the methyl 4-hydroxy-2-[(4-nitrophenyl)sulfanyl]butanoate (8.8 g, 0.0315 mol) in tetrahydrofuran (100 mL), 6-methyl benzotriazinone (6 g, 0.0378 mol) and triphenyl phosphine (12.3 g, 0.0475 mol) were added under argon atmosphere, cooled to about 0° C. and then DIAD (9.5 g, 0.0475 mol) was added. The reaction mixture was stirred for about 30 minutes. The resultant mixture was extracted in ethyl acetate, dried the organic layer with sodium sulphate, concentrated, purified by silica gel column using 7% ethyl acetate/hexane as eluent to obtain the title compound.

Yield: 10.0 g

### Step 3: Synthesis of methyl 2-[(4-aminophenyl)sulfanyl]-4-(6-methyl-4-oxo-1,2,3-benzotriazin-3(4H)-yl)butanoate

To the solution of methyl 4-(6-methyl-4-oxo-1,2,3-benzotriazin-3(4H)-yl)-2-[(4-nitrophenyl)sulfanyl]butanoate (10.0 g, 0.025 mol) in tetrahydrofuran (100 mL)/methanol (100 mL), Pd/C (10 gm) was added. Vacuum and subsequently hydrogen pressure was applied by balloon. The reaction mixture was stirred for about 5 hours. The resultant mixture was filtered through celite and concentrated to obtain the title compound.

Yield: 4.5 g

### Step 4: Synthesis of methyl 4-(6-methyl-4-oxo-1,2,3-benzotriazin-3(4H)-yl)-2-[(4-{[(4-methylphenyl)carbonyl]amino}phenyl)sulfanyl]butanoate

To a solution of methyl 2-[(4-aminophenyl)sulfanyl]-4-(6-methyl-4-oxo-1,2,3-benzotriazin-3(4H)-yl)butanoate (0.200 g, 0.00050 mol) in dichloromethane (10 mL), pyridine (0.113 g, 0.0016 mol) was added under argon atmosphere, cooled to about 0° C., and then p-anisoyl chloride (0.101 g, 0.00059 mol) was added slowly dropwise. Workup done by adding water and extracting in dichloromethane, washed with sodium bicarbonate, dried the organic layer with sodium sulphate and concentrated, purified by 60-120 silica gel column using 15% ethyl acetate/hexane as eluent to obtain the title compound.

Yield: 0.16 g

### Step 5: Synthesis of 4-(6-methyl-4-oxo-1,2,3-benzotriazin-3(4H)-yl)-2-[(4-{[(4-methylphenyl)carbonyl]amino}phenyl)sulfanyl]butanoic acid

To a solution of methyl 4-(6-methyl-4-oxo-1,2,3-benzotriazin-3(4H)-yl)-2-[(4-{[(4-methylphenyl)carbonyl]amino}phenyl)sulfanyl]butanoate (0.150 g, 0.00028 mol) in tetrahydrofuran (5 mL)/methanol (5 mL), a solution of lithium hydroxide (0.018 g, 0.00043 mol) in water was added. The reaction mixture was stirred for about 1 hour. The resultant mixture was acidified with sodium bisulphite solution then extracted in ethyl acetate, dried the organic layer with sodium sulphate and concentrated, purified by preparative TLC and eluted in 10% methanol/dichloromethane to obtain the title compound.

Yield: 0.1 g

LCMS (m/z): 503.03 (M−1)

NMR (DMSO-d₆, 400 MHz): δ 10.17 (1H, s), 8.02-8.08 (2H, dd, J=8 Hz), 7.93-7.95 (2H, d, J=8 Hz), 7.86-7.88 (1H, d, J=8 Hz), 7.72-7.74 (2H, d, J=8 Hz), 7.40-7.42 (2H, d, J=8 Hz), 7.04-7.06 (2H, d, J=8 Hz), 4.49-4.53 (2H, t, J=8 Hz), 3.82 (3H, s), 3.73-3.77 (1H, m), 2.49-2.52 (3H, s), 2.26-2.28 (1H, m), 2.11-2.14 (1H, m).

### Synthetic Procedure for Scheme VII

### Example 15

### Synthesis of 2-[(4-{[(4-methylphenyl)carbonyl]amino}phenyl)sulfonyl]-4-(6-methyl-4-oxo-1,2,3-benzotriazin-3(4H)-yl)butanoic acid (Compound no. 200)

To a solution of 2-[(4-{[(4-methylphenyl)carbonyl]amino}phenyl)sulfanyl]-4-(6-methyl-4-oxo-1,2,3-benzotriazin-3(4H)-yl)butanoic acid (0.100 g, 0.00020 mol) in chloroform (10 mL), metachloroperbenzoic acid (mcpba) (0.139 g, 0.00081 mol) was added and the reaction mixture was cooled to about 0° C. and stirred for about 1 hour. The resultant mixture was acidified using sodium meta bisulphite solution, extracted in dichloromethane, dried the organic layer with sodium sulphate, concentrated, purified by preperative TLC and eluted in 10% methanol/dichloromethane to obtain the title compound.

Yield: 0.05 g

LCMS (m/z): 519.00 (M−1)

NMR (DMSO-d₆, 400 MHz): δ 10.51 (1H, s), 8.00-8.06 (2H, d, J=8 Hz), 7.84-7.93 (6H, m), 7.72-7.74 (2H, d, J=8 Hz), 7.33-7.35 (2H, d, J=8 Hz), 4.39-4.42 (2H, m), 3.81 (1H, m), 2.48-2.50 (3H, s), 2.38 (3H, s), 2.28 (2H, m).

The following compounds were prepared by following above synthetic route:
4-(6-Methoxy-4-oxo-1,2,3-benzotriazin-3(4H)-yl)-2-[(4-{[(4-methylphenyl)carbonyl]amino}phenyl)sulfonyl]butanoic acid (Compound no. 189)

Mass: 535.15
4-(8-Methyl-4-oxo-1,2,3-benzotriazin-3(4H)-yl)-2-[(4-{[(4-methylphenyl)carbonyl]amino}phenyl)sulfonyl]butanoic acid (Compound no. 190)

Mass: 519.18
4-(7-Methyl-4-oxo-1,2,3-benzotriazin-3(4H)-yl)-2-[(4-{[(4-methylphenyl)carbonyl]amino}phenyl)sulfonyl]butanoic acid (Compound no. 201)

Mass: 519.12
2-[(4-{[(4-Methoxyphenyl)carbonyl]amino}phenyl)sulfonyl]-4-(6-methyl-4-oxo-1,2,3-benzotriazin-3(4H)-yl)butanoic acid (Compound no. 206)

Mass: 534.97
2-[(4-{[(3,4-Dichlorophenyl)carbonyl]amino}phenyl)sulfonyl]-4-(6-methyl-4-oxo-1,2,3-benzotriazin-3(4H)-yl)butanoic acid (Compound no. 207)

Mass: 576.02
2-[(4-{[(4-Ethylphenyl)carbonyl]amino}phenyl)sulfonyl]-4-(6-methyl-4-oxo-1,2,3-benzotriazin-3(4H)-yl)butanoic acid (Compound no. 208)

Mass: 534.18
2-[(4-{[(4-Fluorophenyl)carbonyl]amino}phenyl)sulfonyl]-4-(6-methyl-4-oxo-1,2,3-benzotriazin-3(4H)-yl)butanoic acid (Compound no. 209)

Mass: 522.96 (M−2)
2-[(4-{[(4-Chlorophenyl)carbonyl]amino}phenyl)sulfonyl]-4-(6-methyl-4-oxo-1,2,3-benzotriazin-3(4H)-yl)butanoic acid (Compound no. 210)

Mass: 540.98

### Synthetic Procedure for Scheme VIII

### Example 16

### Synthesis of 2-({2-[4-(benzyloxy)phenyl]ethyl}sulfonyl)-4-(4-oxo-1,2,3-benzotriazin-3(4H)-yl)butanoic acid (Compound no. 92)

### Step 1: Synthesis of methyl({2-[4-(benzyloxy)phenyl]ethyl}sulfonyl)acetate

To a solution of methyl({2-[4-(benzyloxy)phenyl]ethyl}sulfanyl)acetate (27 g, 0.085 mol) in methanol (450 mL), a solution of oxone (157 g, 0.256 mol) in water (600 mL) was added and the reaction mixture was stirred at room temperature (˜25° C.) overnight. After completion of reaction, the crude compound was extracted in ethyl acetate and washed with water, purified by 60-120 mesh size silica gel column and eluted in 30% ethyl acetate/hexane to obtain the title compound.

Yield: 26 g.

Mass: 347.45 (M−1)

### Step 2: Synthesis of methyl 2-({2-[4-(benzyloxy)phenyl]ethyl}sulfonyl)-4-(4-oxo-1,2,3-benzotriazin-3(4H)-yl)butanoate

To a solution of methyl({2-[4-(benzyloxy)phenyl]ethyl}sulfonyl)acetate (11 g, 0.0315 mol) in dimethylformamide (157 mL) under argon atmosphere, potassium carbonate (8.72 g, 0.0631 mol), TBAI (3.5 g, 0.009 mol) and benzotrizinone ethyl bromide (8.01 g, 0.0315 mol) were added. The resultant reaction mixture was heated to about 50° C. and stirred for about 4 hours. On completion, water was added and extracted in ethyl acetate, dried the organic layer with sodium sulphate and concentrated, purified by 60-120 silica gel column and eluted at 8% ethyl acetate/hexane to obtain the pure title compound.

Yield: 7.3 g

### Step 3: Synthesis of 2-({2-[4-(benzyloxy)phenyl]ethyl}sulfonyl)-4-(4-oxo-1,2,3-benzotriazin-3(4H)-yl)butanoic acid

To a solution of methyl 2-({2-[4-(benzyloxy)phenyl]ethyl}sulfonyl)-4-(4-oxo-1,2,3-benzotriazin-3(4H)-yl)butanoate (0.2 g, 0.38 mmol) in tetrahydrofuran (3 ml)/methanol (1 mL), a solution of lithium hydroxide (0.016 g, 0.38 mmol) in water was added and the reaction mixture was stirred for about 1 hour. On completion of reaction, sodium bisulphite solution was added to acidify and then extracted in ethyl acetate, dried the organic layer with sodium sulphate and concentrated, purified by preparative TLC eluted in 10% methanol/dichloromethane to obtain the pure title compound.

Yield: 83 mg

Mass: 506.37 (M−1)

NMR (DMSO-d₆, 400 MHz): δ 8.27-8.19 (2H, m), 8.091 (1H, t, J=7.2 Hz), 7.935 (1H, t, J=7.6 Hz), 7.44-7.32 (5H, m), 7.17-6.94 (4H, m), 5.07 (2H, s) 4.54 (2H, m), 3.75 (2H, m), 3.51-3.35 (1H, m), 2.97 (2H, m), 2.51 (2H, m).

### Synthetic Procedure for Scheme IX

### Example 17

### Synthesis of 2-(3-fluorobenzyl)-2-[(2-{4-[(3-fluorobenzyl)oxy]phenyl}ethyl)sulfonyl]-4-(4-oxo-1,2,3-benzotriazin-3(4H)-yl)butanoic acid (Compound no. 191)

### Step 1: Synthesis of methyl 2-{[2-(4-hydroxyphenyl)ethyl]sulfonyl}-4-(4-oxo-1,2,3-benzotriazin-3(4H)-yl)butanoate

To a solution of methyl 2-({2-[4-(benzyloxy)phenyl]ethyl}sulfonyl)-4-(4-oxo-1,2,3-benzotriazin-3(4H)-yl)butanoate (0.469 g, 0.9 mmol) in dichloromethane (12 mL), dimethylsulphide (0.33 mL, 0.00450 mol) and boron trifluoride etherate (0.580 g, 0.00450 mol) were added at room temperature and stirred for about 4 days. The reaction mixture was then extracted in dichloromethane, washed with water and purified on preparative TLC run in 50% ethyl acetate/hexane and eluted in 10% methanol/dichloromethane to contain the title product.

Yield: 100 mg

Mass: 432.40 (M+1)

### Step 2: Synthesis of methyl 2-(3-fluorobenzyl)-2-[(2-{4-[(3-fluorobenzyl)oxy]phenyl}ethyl)sulfonyl]-4-(4-oxo-1,2,3-benzotriazin-3(4H)-yl)butanoate

Potassium carbonate (0.192 g, 0.139 mol) and 3-fluorobenzyl bromide (0.096 mL, 0.765 mol) were added to a solution of methyl 2-{[2-(4-hydroxyphenyl)ethyl]sulfonyl}-4-(4-oxo-1,2,3-benzotriazin-3(4H)-yl)butanoate (0.3 g, 0.639 mmol) in N,N-dimethylformamide (2 mL) at room temperature and this was stirred at ambient conditions overnight. After completion of reaction, the reaction mixture was extracted in ethyl acetate and washed with water and saturated brine solution. The crude compound was purified in a silica gel column chromatograph and eluted with 20% ethyl acetate in hexane to give the title compound.

Yield: 450 mg

### Step 3: Synthesis of 2-(3-fluorobenzyl)-2-[(2-{4-[(3-fluorobenzyl)oxy]phenyl}ethyl)sulfonyl]-4-(4-oxo-1,2,3-benzotriazin-3(4H)-yl)butanoic acid

To a solution of methyl 2-(3-fluorobenzyl)-2-[(2-{4-[(3-fluorobenzyl)oxy]phenyl}ethyl)sulfonyl]-4-(4-oxo-1,2,3-benzotriazin-3(4H)-yl)butanoate (0.452 g, 0.838 mmol) in tetrahydrofuran (1 mL) and methanol (2 mL), a solution of lithium hydroxide (0.028 g, 0.683 mmol) in water was added and the reaction mixture was stirred overnight at room temperature (˜25° C.). To the resultant mixture, sodium bisulphite solution was added to acidify and then extracted in ethyl acetate. The organic layer was dried with sodium sulphate and concentrated and purified by preperative TLC with 10% methanol/dichloromethane to obtain the title product.

Yield: 120 mg

Mass: 632.25 (M−1)

NMR (DMSO-d₆, 400 MHz): δ 8.24-8.17 (2H, dd, J=7.6 Hz & 8 Hz), 8.07 (1H, t, J=7.6 Hz), 7.92 (1H, t, J=7.2 Hz), 7.442 (2H, d, J=6.4 Hz), 7.29-7.16 (8H, m), 6.98 (2H, t, J=8 Hz), 5.12 (2H, s), 4.73 (2H, s), 3.66-3.37 (2H, m), 3.23-3.19 (2H, m), 2.99 (2H, m), 2.51-2.34 (2H, m).

The following compounds were synthesized by following the similar route of synthesis:
2-(4-Fluorobenzyl)-2-[(2-{4-[(4-fluorobenzyl)oxy]phenyl}ethyl)sulfonyl]-4-(4-oxo-1,2,3-benzotriazin-3(4H)-yl)butanoic acid (Compound no. 186)

Mass: 632.25 (M−1)
2-(2-Chlorobenzyl)-2-[(2-{4-[(2-chlorobenzyl)oxy]phenyl}ethyl)sulfonyl]-4-(4-oxo-1,2,3-benzotriazin-3(4H)-yl)butanoic acid (Compound no. 198)

Mass: 664.12 (M−2, Cl pattern)

### Example

### Assay for Matrix Metallo Proteinases (MMPs)

New chemical entities of the present invention and corresponding standards used in the present invention were prepared (stock 10 mM) in 100% DMSO and subsequent dilutions were made in [MMP assay buffer: 50 mM HEPES, 10 mM CaCl2, 150 nM NaCl, 1 μM zinc acetate, 600 μM CHAPS (pH 7.4). Assays used human MMPs expressed either as full length or catalytic domain. The Collagenase (MMP-1), Gelatinase (MMP-9), Elastase (MMP-12) and membrate type-1 (MMP-14) were cleaved and activated using reagent APMA (4-amino phenyl mercuric acetate) to obtain active catalytic domains. In a typical 100 μl reaction assay mixture, 1.0 μl of desired MMP enzyme was incubated in buffered solution in absence or presence of 1.0 μl of NCEs/standards for 30 minutes. Reaction was started with desired fluorogenic substrate—FAMTAMRA (FAM-Thr-Pro-Gly-Pro-Leu-Gly-Leu-Dpa-Ala-Arg-Arg-Lys-TAMRA-NH2) at a final concentration of 10 μM per well and reaction was allowed to proceed for 45 minutes and rate of velocity was monitored (increase in RFUs) at Exc 495 nm and emi 525 nm. Blank reaction rate (without enzyme) was subtracted from each value. The % control activity=(inhibited rate/control rate)×100. IC₅₀ values were calculated using least square regression analsysis method by Graph-Pad prism version 4.2 software, using a 5-6 point dose response curve in presence of inhibitor. IC₅₀ values were averaged for duplicate assay data and values were tabulated. The present invention relates to compounds that act as dual MMP-9/12 inhibitors, which have desirable activity profiles.

MMP9 activities of the compounds disclosed in the invention provided IC₅₀ values from about 10 micromolar to about 2.6 nM, or from about 1 micromolar to about 2.6 nM, or from about 650 nM to about 2.6 nM, or from about 300 nM to about 2.6 nM, or from about 100 nM to about 2.6 nM, or from about 50 nM to about 2.6 nM, or from about 30 nM to about 2.6 nM, or from about 20 nM to about 2.6 nM, or from about 12 to about 2.6 nM, as compared to about −1.4 to 3.2 nM for marimastat.

MMP 12 activities of the compounds disclosed in the invention provided IC₅₀ values from about 10 micromolar to about 0.13 nM, or from about 1 micromolar to about 0.13 nM, or from about 300 nM to about 0.13 nM, or from about 100 nM to about 0.13 nM, or from about 50 nM to about 0.13 nM, or from about 30 nM to about 0.13 nM, or from about 20 nM to about 0.13 nM, or from about 15 nM to about 0.13 nM, or from about 7 to about 0.13 nM as compared to to 0.2 nM to 0.9 nM for marimastat.

We claim:

1. A compound of Formula I 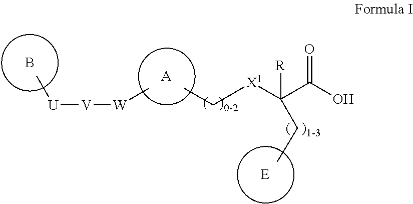
including racemates, enantiomers and diastereomers thereof, or a pharmaceutically acceptable salt thereof; wherein, 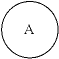
represents (un)substituted aryl or heteroaryl; 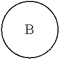
represents C₆-C₁₂ aryl, C₃-C₁₂ cycloalkyl, C₆-C₁₂ heteroaryl or C₆-C₁₂ heterocyclyl each of which is optionally further substituted by one or more substituents independently selected from R¹;
U represents bond, —NH—, —C(═O)—, —(CH₂)ₙ—, —C(═S)—, —O—, —SO₂— or —S— wherein n represents zero or an integer between 1 and 2;
V represents bond, —NH—, —C(═O)—, —C(═S)— or —SO₂—;
W represents bond, —NH—, —C(═O)—, (CH₂)ₙ—, —C(═S)—, —O—, —S— or —SO₂—;
X¹ represents-O—, —S—, —SO— or —SO₂—;
R represents H, alkyl or arylalkyl;
R¹ represent salkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙ NHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl, n is as defined earlier and m is an integer 0-2}; 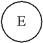
represents mono-, bi- or polycyclic heteroaryl or heterocyclyl selected from the following: 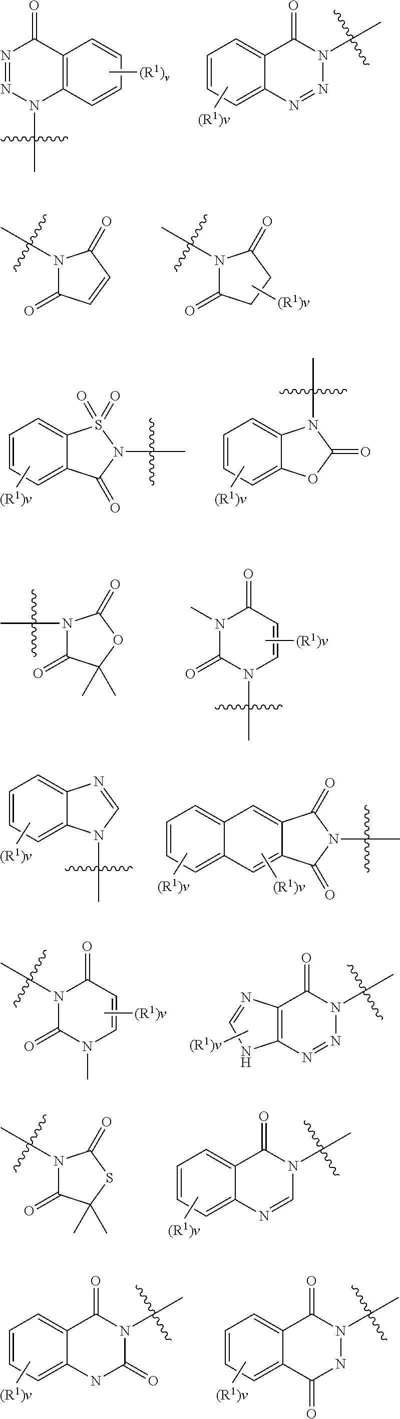 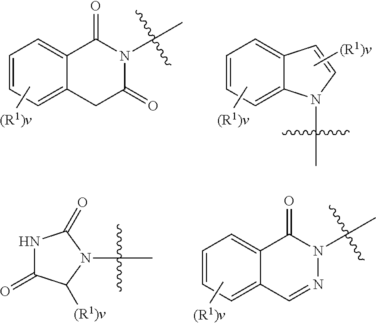
wherein R¹ is as defined earlier and v represents zero or an integer between 1-4.

2. A compound according to claim, having the structure of Formula Ia 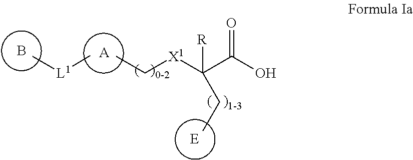
including racemates, enantiomers and diastereomers thereof, or a pharmaceutically acceptable salt thereof wherein, 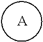
represents (un)substituted aryl or heteroaryl; 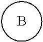
represents C₆-C₁₂ aryl, C₃-C₁₂ cycloalkyl, C₆-C₁₂ heteroaryl or C₆-C₁₂ heterocyclyl each of which is optionally further substituted by one or more substituents independently selected from R¹;
L¹ represents bond, —(CH₂)ₙ—, —NHC(═O)(CH₂)ₙ—, —(CH₂)ₙC(═O)NH—, —NHC(═O)NH—, —SO₂NH—, —NHSO₂, —SO₂, —NHC(═O)(O)—, —O—(CH₂)ₙ—, —(CH₂)ₙ—O—, —(CH₂)ₙOC(═O)NH—, —C(═S)NH—, —NHC(═S)— or —NHC(═S)NH— wherein n represent zero or an integer between 1 and 2;
X¹ represents —O—, —S—, —SO— or —SO₂—;
R represents H, alkyl or arylalkyl;
R¹ represents alkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙ NHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl, n is as defined earlier and m is an integer 0-2}; 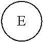
represents mono-, bi- or polycyclic heteroaryl or heterocyclyl selected from the following 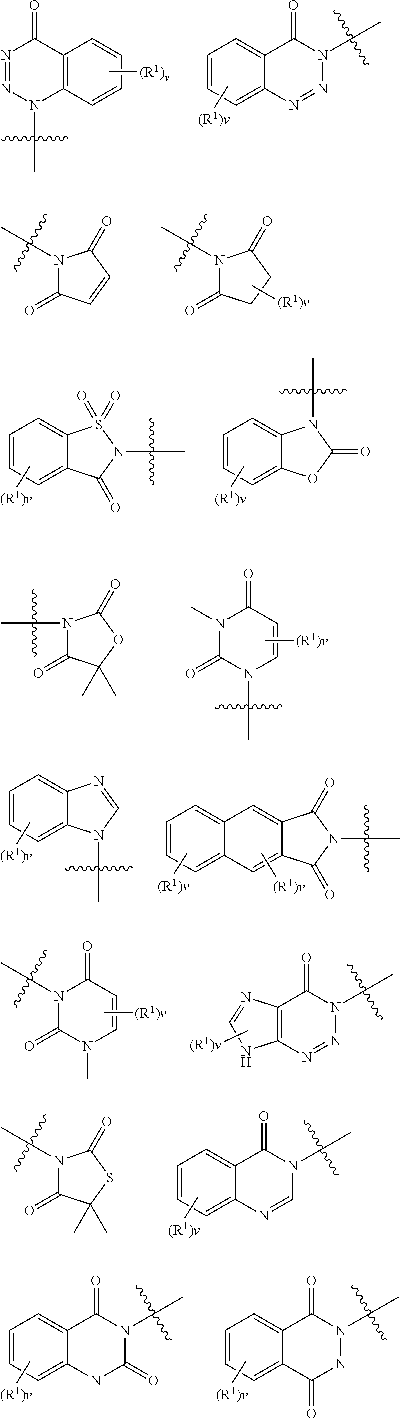 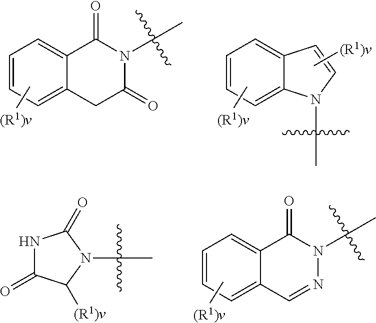
wherein R¹ is as defined earlier and v represents zero or an integer between 1-4.

3. A compound of Formula I, which is:
2-[(4-{[(4-Methylphenyl)carbonyl]amino}phenyl)sulfonyl]-4-(4-oxo-1,2,3-benzotriazin-3(4H)-yl)butanoic acid (Compound no. 1),
2-[(3′-Methoxybiphenyl-4-yl) sulfonyl]-4-(4-oxo-1,2,3-benzotriazin-3(4H)-yl)butanoic acid (Compound no. 2),
2-[(3′-Fluoro-4′-methoxybiphenyl-4-yl)sulfonyl]-4-(6-methyl-4-oxo-1,2,3-benzotriazin-3(4H)-yl)butanoic acid (Compound no. 3),
2-{[2-(4′-Chlorobiphenyl-4-yl)ethyl]sulfonyl}-4-(4-oxo-1,2,3-benzotriazin-3(4H)-yl)butanoic acid (Compound no. 4),
2-{[2-(4′-Chlorobiphenyl-4-yl)ethyl]sulfonyl}-4-(6-fluoro-4-oxo-1,2,3-benzotriazin-3(4H)-yl)butanoic acid (Compound no. 5),
2-{[(4′-Chlorobiphenyl-4-yl)methyl]sulfonyl}-4-(7-methyl-4-oxo-1,2,3-benzotriazin-3(4H)-yl)butanoic acid (Compound no. 6),
2-{[(4′-Chlorobiphenyl-4-yl)methyl]sulfonyl}-4-(4-oxo-1,2,3-benzotriazin-3(4H)-yl)butanoic acid (Compound no. 7),
2-[(3′,4′-Difluorobiphenyl-4-yl)sulfonyl]-4-(6-methyl-4-oxo-1,2,3-benzotriazin-3(4H)-yl)butanoic acid (Compound no. 8),
2-{[(4′-Chlorobiphenyl-4-yl)methyl]sulfonyl}-4-[4-oxo-7-(trifluoromethyl)-1,2,3-benzotriazin-3(4H)-yl]butanoic acid (Compound no. 9),
4-(6-Methyl-4-oxo-1,2,3-benzotriazin-3(4H)-yl)-2-{[4′-(trifluoromethyl)biphenyl-4-yl]sulfonyl}butanoic acid (Compound no. 10),
2-[(3′,4′-Dichlorobiphenyl-4-yl)sulfonyl]-4-(6-methyl-4-oxo-1,2,3-benzotriazin-3(4H)-yl)butanoic acid (Compound no. 11),
2-[(4′-Methoxy-3′-methylbiphenyl-4-yl)sulfonyl]-4-(6-methyl-4-oxo-1,2,3-benzotriazin-3(4H)-yl)butanoic acid (Compound no. 12),
2-[(3′,4′-Difluorobiphenyl-4-yl)sulfonyl]-4-(6-methyl-4-oxo-1,2,3-benzotriazin-3(4H)-yl)butanoic acid (Compound no. 13),
2-[(3′-Fluoro-4′-methylbiphenyl-4-yl)sulfonyl]-4-(6-methyl-4-oxo-1,2,3-benzotriazin-3(4H)-yl)butanoic acid (Compound no. 14),
2-[(4′-Fluorobiphenyl-4-yl)sulfonyl]-4-(6-methyl-4-oxo-1,2,3-benzotriazin-3(4H)-yl)butanoic acid (Compound no. 15),
2-[(4′-Chlorobiphenyl-4-yl)sulfonyl]-4-(6-methyl-4-oxo-1,2,3-benzotriazin-3(4H)-yl)butanoic acid (Compound no. 16),
2-[(4′-Ethylbiphenyl-4-yl)sulfonyl]-4-(6-methyl-4-oxo-1,2,3-benzotriazin-3(4H)-yl)butanoic acid (Compound no. 17),
2-[(4′-Methoxybiphenyl-4-yl)sulfonyl]-4-(6-methyl-4-oxo-1,2,3-benzotriazin-3(4H)-yl)butanoic acid (Compound no. 18),
2-[(4′-Fluoro-3′-methylbiphenyl-4-yl)sulfonyl]-4-(6-methyl-4-oxo-1,2,3-benzotriazin-3(4H)-yl)butanoic acid (Compound no. 19),
2-{[4-(6-Methoxypyridin-3-yl)benzyl]sulfonyl}-4-(4-oxo-1,2,3-benzotriazin-3(4H)-yl)butanoic acid (Compound no. 20),
4-(4-Oxo-1,2,3-benzotriazin-3(4H)-yl)-2-({[4′-(trifluoromethoxy)biphenyl-4-yl]methyl}sulfonyl)butanoic acid (Compound no. 21),
2-{[(3′,4′-Dimethoxybiphenyl-4-yl)methyl]sulfonyl}-4-(4-oxo-1,2,3-benzotriazin-3(4H)-yl)butanoic acid (Compound no. 22),
2-{[(3′-Fluoro-4′-methylbiphenyl-4-yl)methyl]sulfonyl}-4-(4-oxo-1,2,3-benzotriazin-3(4H)-yl)butanoic acid (Compound no. 23),
2-{[(3′,4′-Dimethylbiphenyl-4-yl)methyl]sulfonyl}-4-(4-oxo-1,2,3-benzotriazin-3(4H)-yl)butanoic acid (Compound no. 24),
2-{[(3′,4′-Dichlorobiphenyl-4-yl)methyl]sulfonyl}-4-(4-oxo-1,2,3-benzotriazin-3(4H)-yl)butanoic acid (Compound no. 25),
2-{[(4′-Fluoro-3′-methylbiphenyl-4-yl)methyl]sulfonyl}-4-(4-oxo-1,2,3-benzotriazin-3(4H)-yl)butanoic acid (Compound no. 26),
4-(4-Oxo-1,2,3-benzotriazin-3(4H)-yl)-2-({[4′-(trifluoromethyl)biphenyl-4-yl]methyl}sulfonyl)butanoic acid (Compound no. 27),
2-{[(4′-Methoxybiphenyl-4-yl)methyl]sulfonyl}-4-(4-oxo-1,2,3-benzotriazin-3(4H)-yl)butanoic acid (Compound no. 28),
2-{[(4′-Fluorobiphenyl-4-yl)methyl]sulfonyl}-4-(4-oxo-1,2,3-benzotriazin-3(4H)-yl)butanoic acid (Compound no. 29),
2-({2-[4-(6-Methoxypyridin-3-yl)phenyl]ethyl}sulfonyl)-4-(4-oxo-1,2,3-benzotriazin-3(4H)-yl)butanoic acid (Compound no. 30),
2-{[2-(3′-Fluoro-4′-methylbiphenyl-4-yl)ethyl]sulfonyl}-4-(4-oxo-1,2,3-benzotriazin-3(4H)-yl)butanoic acid (Compound no. 31),
2-{[2-(4′-Ethylbiphenyl-4-yl)ethyl]sulfonyl}-4-(4-oxo-1,2,3-benzotriazin-3(4H)-yl)butanoic acid (Compound no. 32),
2-{[2-(3′,4′-Difluorobiphenyl-4-yl)ethyl]sulfonyl}-4-(4-oxo-1,2,3-benzotriazin-3(4H)-yl)butanoic acid (Compound no. 33),
2-{[2-(4′-Cyanobiphenyl-4-yl)ethyl]sulfonyl}-4-(4-oxo-1,2,3-benzotriazin-3(4H)-yl)butanoic acid (Compound no. 34),
2-{[2-(3′-Fluoro-4′-methoxybiphenyl-4-yl)ethyl]sulfonyl}-4-(4-oxo-1,2,3-benzotriazin-3(4H)-yl)butanoic acid (Compound no. 35),
2-({2-[4-(1-Methyl-1H-pyrazol-4-yl)phenyl]ethyl}sulfonyl)-4-(4-oxo-1,2,3-benzotriazin-3(4H)-yl)butanoic acid (Compound no. 36),
2-{[2-(4′-Methylbiphenyl-4-yl)ethyl]sulfonyl}-4-(4-oxo-1,2,3-benzotriazin-3(4H)-yl)butanoic acid (Compound no. 37),
4-(4-Oxo-1,2,3-benzotriazin-3(4H)-yl)-2-({2-[4′-(trifluoromethoxy)biphenyl-4-yl]ethyl}sulfonyl)butanoic acid (Compound no. 38),
4-(7-Methyl-4-oxo-1,2,3-benzotriazin-3(4H)-yl)-2-{[4′-(trifluoromethoxy)biphenyl-4-yl]sulfonyl}butanoic acid (Compound no. 39),
4-(6-Methoxy-4-oxo-1,2,3-benzotriazin-3(4H)-yl)-2-{[4′-(trifluoromethoxy)biphenyl-4-yl]sulfonyl}butanoic acid (Compound no. 40),
4-(7-Methoxy-4-oxo-1,2,3-benzotriazin-3(4H)-yl)-2-{[4′-(trifluoromethoxy)biphenyl-4-yl]sulfonyl}butanoic acid (Compound no. 41),
2-[(4′-tert-Butylbiphenyl-4-yl)sulfonyl]-4-(4-oxo-1,2,3-benzotriazin-3(4H)-yl)butanoic acid (Compound no. 42),
2-[(4′-Ethylbiphenyl-4-yl)sulfonyl]-4-(4-oxo-1,2,3-benzotriazin-3(4H)-yl)butanoic acid (Compound no. 43),
4-(4-Oxo-1,2,3-benzotriazin-3(4H)-yl)-2-{[4′-(propan-2-yl)biphenyl-4-yl]sulfonyl}butanoic acid (Compound no. 44),
4-(4-Oxo-1,2,3-benzotriazin-3(4H)-yl)-2-{[4′-(trifluoromethoxy)biphenyl-4-yl]sulfonyl}butanoic acid (Compound no. 45),
4-(4-Oxo-1,2,3-benzotriazin-3 (4H)-yl)-2-({4-[(phenylcarbonyl)amino]phenyl}sulfonyl)butanoic acid (Compound no. 46),
2-[(4-{[(4-Methoxyphenyl)carbonyl]amino}phenyl)sulfonyl]-4-(4-oxo-1,2,3-benzotriazin-3(4H)-yl)butanoic acid (Compound no. 47),
4-(7-Methoxy-4-oxo-1,2,3-benzotriazin-3(4H)-yl)-2-{[4′-(trifluoromethyl)biphenyl-4-yl]sulfonyl}butanoic acid (Compound no. 48),
2-[(4′-Methoxybiphenyl-4-yl)sulfonyl]-4-(7-methoxy-4-oxo-1,2,3-benzotriazin-3(4H)-yl)butanoic acid (Compound no. 49),
2-[(3′-Fluoro-4′-methylbiphenyl-4-yl)sulfonyl]-4-(7-methoxy-4-oxo-1,2,3-benzotriazin-3(4H)-yl)butanoic acid (Compound no. 50),
2-[(3′,4′-Dimethoxybiphenyl-4-yl)sulfonyl]-4-(7-methoxy-4-oxo-1,2,3-benzotriazin-3(4H)-yl)butanoic acid (Compound no. 51),
2-[(4-{[(3-Methoxyphenyl)carbonyl]amino}phenyl)sulfonyl]-4-(4-oxo-1,2,3-benzotriazin-3(4H)-yl)butanoic acid (Compound no. 52),
2-[(4-{[(3-Fluorophenyl)carbonyl]amino}phenyl)sulfonyl]-4-(4-oxo-1,2,3-benzotriazin-3(4H)-yl)butanoic acid (Compound no. 53),
2-[(4-{[(4-Fluorophenyl)carbonyl]amino}phenyl)sulfonyl]-4-(4-oxo-1,2,3-benzotriazin-3(4H)-yl)butanoic acid (Compound no. 54),
2-[(4-{[(4-Chlorophenyl)carbonyl]amino}phenyl)sulfonyl]-4-(4-oxo-1,2,3-benzotriazin-3(4H)-yl)butanoic acid (Compound no. 55),
2-[(4′-Ethylbiphenyl-4-yl)sulfonyl]-4-(7-methoxy-4-oxo-1,2,3-benzotriazin-3(4H)-yl)butanoic acid (Compound no. 56),
2-[(4′-Chlorobiphenyl-4-yl)sulfonyl]-4-(7-methoxy-4-oxo-1,2,3-benzotriazin-3(4H)-yl)butanoic acid (Compound no. 57),
2-[(3′,4′-Dichlorobiphenyl-4-yl)sulfonyl]-4-(7-methoxy-4-oxo-1,2,3-benzotriazin-3(4H)-yl)butanoic acid (Compound no. 58),
2-[(3′-Fluoro-4′-methoxybiphenyl-4-yl)sulfonyl]-4-(7-methoxy-4-oxo-1,2,3-benzotriazin-3(4H)-yl)butanoic acid (Compound no. 59),
2-[(3′,4′-Difluorobiphenyl-4-yl)sulfonyl]-4-(6-methoxy-4-oxo-1,2,3-benzotriazin-3(4H)-yl)butanoic acid (Compound no. 60),
2-[(3′-Fluoro-4′-methylbiphenyl-4-yl)sulfonyl]-4-(6-methoxy-4-oxo-1,2,3-benzotriazin-3(4H)-yl)butanoic acid (Compound no. 61),
2-[(4′-Fluoro-3′-methylbiphenyl-4-yl)sulfonyl]-4-(6-methoxy-4-oxo-1,2,3-benzotriazin-3(4H)-yl)butanoic acid (Compound no. 62),
2-[(3′,4′-Dichlorobiphenyl-4-yl)sulfonyl]-4-(6-methoxy-4-oxo-1,2,3-benzotriazin-3(4H)-yl)butanoic acid (Compound no. 63),
2-[(3′-Fluoro-4′-methoxybiphenyl-4-yl)sulfonyl]-4-(6-methoxy-4-oxo-1,2,3-benzotriazin-3(4H)-yl)butanoic acid (Compound no. 64),
2-{[4′-Chloro-3′-(trifluoromethyl)biphenyl-4-yl]sulfonyl}-4-(6-methoxy-4-oxo-1,2,3-benzotriazin-3(4H)-yl)butanoic acid (Compound no. 65),
2-[(4′-Ethylbiphenyl-4-yl)sulfonyl]-4-(6-methoxy-4-oxo-1,2,3-benzotriazin-3(4H)-yl)butanoic acid (Compound no. 66),
2-[(3′,4′-Dimethylbiphenyl-4-yl)sulfonyl]-4-(6-methoxy-4-oxo-1,2,3-benzotriazin-3(4H)-yl)butanoic acid (Compound no. 67),
4-(6-Methoxy-4-oxo-1,2,3-benzotriazin-3(4H)-yl)-2-[(4′-methylbiphenyl-4-yl)sulfonyl]butanoic acid (Compound no. 68),
2-[(4′-Chlorobiphenyl-4-yl)sulfonyl]-4-(6-methoxy-4-oxo-1,2,3-benzotriazin-3(4H)-yl)butanoic acid (Compound no. 69),
4-(6-Methoxy-4-oxo-1,2,3-benzotriazin-3(4H)-yl)-2-{[4′-(trifluoromethyl)biphenyl-4-yl]sulfonyl}butanoic acid (Compound no. 70),
2-[(4′-Methoxybiphenyl-4-yl)sulfonyl]-4-(6-methoxy-4-oxo-1,2,3-benzotriazin-3(4H)-yl)butanoic acid (Compound no. 71),
4-(4-Oxo-1,2,3-benzotriazin-3(4H)-yl)-2-({2-[4-({[4-(trifluoromethyl)phenyl]carbonyl}amino)phenyl]ethyl}sulfonyl)butanoic acid (Compound no. 72),
4-(4-Oxo-1,2,3-benzotriazin-3(4H)-yl)-2-({2-[4-({[4-(trifluoromethoxy)phenyl]carbonyl}amino)phenyl]ethyl}sulfonyl)butanoic acid (Compound no. 73),
2-[(4-{[(3,4-Dichlorophenyl)carbonyl]amino}phenyl)sulfonyl]-4-(4-oxo-1,2,3-benzotriazin-3(4H)-yl)butanoic acid (Compound no. 74),
2-[(3′-Fluoro-4′-methoxybiphenyl-4-yl)sulfanyl]-4-[4-oxo-7-(trifluoromethyl)-1,2,3-benzotriazin-3(4H)-yl]butanoic acid (Compound no. 75),
4-(4-Oxo-1,2,3-benzotriazin-3(4H)-yl)-2-[(2-{4-[(phenylcarbonyl)amino]phenyl}ethyl)sulfonyl]butanoic acid (Compound no. 76),
4-(4-Oxo-1,2,3-benzotriazin-3(4H)-yl)-2-[(2-{4-[(thiophen-2-ylcarbonyl)amino]phenyl}ethyl)sulfonyl]butanoic acid (Compound no. 77),
2-[(2-{4-[(Cyclopentylcarbonyl)amino]phenyl}ethyl)sulfonyl]-4-(4-oxo-1,2,3-benzotriazin-3(4H)-yl)butanoic acid (Compound no. 78),
2-[(2-{4-[(Cyclopropylcarbonyl)amino]phenyl}ethyl)sulfonyl]-4-(4-oxo-1,2,3-benzotriazin-3(4H)-yl)butanoic acid (Compound no. 79),
2-{[2-(4-{[(3-Methoxyphenyl)carbonyl]amino}phenyl)ethyl]sulfonyl}-4-(4-oxo-1,2,3-benzotriazin-3(4H)-yl)butanoic acid (Compound no. 80),
2-{[2-(4-{[(3-Chlorophenyl)carbonyl]amino}phenyl)ethyl]sulfonyl}-4-(4-oxo-1,2,3-benzotriazin-3(4H)-yl)butanoic acid (Compound no. 81),
2-{[2-(4-{[(3-Fluorophenyl)carbonyl]amino}phenyl)ethyl]sulfonyl}-4-(4-oxo-1,2,3-benzotriazin-3(4H)-yl)butanoic acid (Compound no. 82),
2-{[2-(4-{[(4-Ethylphenyl)carbonyl]amino}phenyl)ethyl]sulfonyl}-4-(4-oxo-1,2,3-benzotriazin-3(4H)-yl)butanoic acid (Compound no. 83),
2-{[2-(4-{[(4-Methoxyphenyl)sulfonyl]amino}phenyl)ethyl]sulfonyl}-4-(4-oxo-1,2,3-benzotriazin-3(4H)-yl)butanoic acid (Compound no. 84),
2-[(4-{[(3-Ethoxyphenyl)carbamoyl]amino}phenyl)sulfonyl]-4-(4-oxo-1,2,3-benzotriazin-3(4H)-yl)butanoic acid (Compound no. 85),
2-{[4-({[2-Fluoro-5-(trifluoromethyl)phenyl]carbamoyl}amino)phenyl]sulfonyl}-4-(4-oxo-1,2,3-benzotriazin-3(4H)-yl)butanoic acid (Compound no. 86),
2-[(4-{[(2,6-Dimethoxyphenyl)carbonyl]amino}phenyl)sulfonyl]-4-(4-oxo-1,2,3-benzotriazin-3(4H)-yl)butanoic acid (Compound no. 87),
2-[(4-{[(4-Fluorophenyl)carbamoyl]amino}phenyl)sulfonyl]-4-(4-oxo-1,2,3-benzotriazin-3(4H)-yl)butanoic acid (Compound no. 88),
2-[(4′-Fluorobiphenyl-4-yl)sulfonyl]-4-(8-methyl-4-oxo-1,2,3-benzotriazin-3(4H)-yl)butanoic acid (Compound no. 89),
2-[(3′-Ffluoro-4′-methylbiphenyl-4-yl)sulfonyl]-4-(8-methyl-4-oxo-1,2,3-benzotriazin-3(4H)-yl)butanoic acid (Compound no. 90),
2-[(4′-Fluoro-3′-methylbiphenyl-4-yl)sulfonyl]-4-(8-methyl-4-oxo-1,2,3-benzotriazin-3(4H)-yl)butanoic acid (Compound no. 91),
2-({2-[4-(Benzyloxy)phenyl]ethyl}sulfonyl)-4-(4-oxo-1,2,3-benzotriazin-3(4H)-yl)butanoic acid (Compound no. 92),
2-[(3′,4′-Dimethylbiphenyl-4-yl)sulfonyl]-4-(8-methyl-4-oxo-1,2,3-benzotriazin-3(4H)-yl)butanoic acid (Compound no. 93),
2-[(4-{[(3-Methylphenyl)carbonyl]amino}phenyl)sulfonyl]-4-(4-oxo-1,2,3-benzotriazin-3(4H)-yl)butanoic acid (Compound no. 94),
2-[(4-{[(2-Methoxyphenyl)carbonyl]amino}phenyl)sulfonyl]-4-(4-oxo-1,2,3-benzotriazin-3(4H)-yl)butanoic acid (Compound no. 95),
2-[(4′-Ethylbiphenyl-4-yl)sulfonyl]-4-(8-methyl-4-oxo-1,2,3-benzotriazin-3(4H)-yl)butanoic acid (Compound no. 96),
2-[(4′-Methylbiphenyl-4-yl)sulfonyl]-4-(8-methyl-4-oxo-1,2,3-benzotriazin-3(4H)-yl)butanoic acid (Compound no. 97),
2-{[4-(6-Methoxypyridin-3-yl)phenyl]sulfonyl}-4-(8-methyl-4-oxo-1,2,3-benzotriazin-3(4H)-yl)butanoic acid (Compound no. 98),
2-({4-[(Cyclohexylcarbonyl)amino]phenyl}sulfonyl)-4-(4-oxo-1,2,3-benzotriazin-3(4H)-yl)butanoic acid (Compound no. 99),
2-[(4-{[(2-Methylphenyl)carbonyl]amino}phenyl)sulfonyl]-4-(4-oxo-1,2,3-benzotriazin-3(4H)-yl)butanoic acid (Compound no. 100),
2-({4-[(Cyclopropylcarbonyl)amino]phenyl}sulfonyl)-4-(4-oxo-1,2,3-benzotriazin-3(4H)-yl)butanoic acid (Compound no. 101),
4-(4-Oxo-1,2,3-benzotriazin-3(4H)-yl)-2-({4-[(thiophen-2-ylcarbonyl)amino]phenyl}sulfonyl)butanoic acid (Compound no. 102),
2-({4-[(Cyclopentylcarbonyl)amino]phenyl}sulfonyl)-4-(4-oxo-1,2,3-benzotriazin-3(4H)-yl)butanoic acid (Compound no. 103),
2-{[4-({[4-Fluoro-3-(trifluoromethyl)phenyl]carbonyl}amino)phenyl]sulfonyl}-4-(4-oxo-1,2,3-benzotriazin-3(4H)-yl)butanoic acid (Compound no. 104),
2-[(3′-Fluoro-4′-methoxybiphenyl-4-yl)sulfanyl]-4-(7-methyl-4-oxo-1,2,3-benzotriazin-3(4H)-yl)butanoic acid (Compound no. 105),
2-[(4′-Chlorobiphenyl-4-yl)sulfanyl]-4-(6-methoxy-4-oxo-1,2,3-benzotriazin-3(4H)-yl)butanoic acid (Compound no. 106),
2-[(4′-Chlorobiphenyl-4-yl)sulfanyl]-4-(6-methyl-4-oxo-1,2,3-benzotriazin-3(4H)-yl)butanoic acid (Compound no. 107),
2-[(3′-Fluoro-4′-methoxybiphenyl-4-yl)sulfanyl]-4-(6-methyl-4-oxo-1,2,3-benzotriazin-3(4H)-yl)butanoic acid (Compound no. 108),
2-[(3′-Fluoro-4′-methoxybiphenyl-4-yl)sulfanyl]-4-(4-oxo-1,2,3-benzotriazin-3(4H)-yl)butanoic acid (Compound no. 109),
4-(6-Methyl-4-oxo-1,2,3-benzotriazin-3(4H)-yl)-2-{[4′-(trifluoromethyl)biphenyl-4-yl]sulfanyl}butanoic acid (Compound no. 110),
2-[(3′,4′-Dichlorobiphenyl-4-yl)sulfanyl]-4-(6-methyl-4-oxo-1,2,3-benzotriazin-3(4H)-yl)butanoic acid (Compound no. 111),
2-[(4′-Methoxy-3′-methylbiphenyl-4-yl)sulfanyl]-4-(6-methyl-4-oxo-1,2,3-benzotriazin-3(4H)-yl)butanoic acid (Compound no. 112),
2-[(3′,4′-Difluorobiphenyl-4-yl)sulfanyl]-4-(6-methyl-4-oxo-1,2,3-benzotriazin-3(4H)-yl)butanoic acid (Compound no. 113),
2-[(3′-Fluoro-4′-methylbiphenyl-4-yl)sulfanyl]-4-(6-methyl-4-oxo-1,2,3-benzotriazin-3(4H)-yl)butanoic acid (Compound no. 114),
2-[(4′-Fluorobiphenyl-4-yl)sulfanyl]-4-(6-methyl-4-oxo-1,2,3-benzotriazin-3(4H)-yl)butanoic acid (Compound no. 115),
2-[(4′-Ethylbiphenyl-4-yl)sulfanyl]-4-(6-methyl-4-oxo-1,2,3-benzotriazin-3(4H)-yl)butanoic acid (Compound no. 116),
2-[(4′-Methoxybiphenyl-4-yl)sulfanyl]-4-(6-methyl-4-oxo-1,2,3-benzotriazin-3(4H)-yl)butanoic acid (Compound no. 117),
2-[(4′-Fluoro-3′-methylbiphenyl-4-yl)sulfanyl]-4-(6-methyl-4-oxo-1,2,3-benzotriazin-3(4H)-yl)butanoic acid (Compound no. 118),
4-(4-Oxo-1,2,3-benzotriazin-3(4H)-yl)-2-{[4′-(trifluoromethyl)biphenyl-4-yl]sulfanyl}butanoic acid (Compound no. 119),
4-(8-Methyl-4-oxo-1,2,3-benzotriazin-3(4H)-yl)-2-{[4′-(trifluoromethoxy)biphenyl-4-yl]sulfanyl}butanoic acid (Compound no. 120),
4-(7-Methyl-4-oxo-1,2,3-benzotriazin-3(4H)-yl)-2-{[4′-(trifluoromethoxy)biphenyl-4-yl]sulfanyl}butanoic acid (Compound no. 121),
4-(6-Methoxy-4-oxo-1,2,3-benzotriazin-3(4H)-yl)-2-{[4′-(trifluoromethoxy)biphenyl-4-yl]sulfanyl}butanoic acid (Compound no. 122),
4-(7-Methoxy-4-oxo-1,2,3-benzotriazin-3(4H)-yl)-2-{[4′-(trifluoromethoxy)biphenyl-4-yl]sulfanyl}butanoic acid (Compound no. 123),
2-[(3′,4′-Difluorobiphenyl-4-yl)sulfanyl]-4-(6-methoxy-4-oxo-1,2,3-benzotriazin-3(4H)-yl)butanoic acid (Compound no. 124),
2-[(3′-Fluoro-4′-methylbiphenyl-4-yl)sulfanyl]-4-(6-methoxy-4-oxo-1,2,3-benzotriazin-3(4H)-yl)butanoic acid (Compound no. 125),
2-[(4′-Fluoro-3′-methylbiphenyl-4-yl)sulfanyl]-4-(6-methoxy-4-oxo-1,2,3-benzotriazin-3(4H)-yl)butanoic acid (Compound no. 126),
2-[(3′,4′-Dichlorobiphenyl-4-yl)sulfanyl]-4-(6-methoxy-4-oxo-1,2,3-benzotriazin-3(4H)-yl)butanoic acid (Compound no. 127),
2-[(3′-Fluoro-4′-methoxybiphenyl-4-yl)sulfanyl]-4-(6-methoxy-4-oxo-1,2,3-benzotriazin-3(4H)-yl)butanoic acid (Compound no. 128),
2-{[4′-chloro-3′-(trifluoromethyl)biphenyl-4-yl]sulfanyl}-4-(6-methoxy-4-oxo-1,2,3-benzotriazin-3(4H)-yl)butanoic acid (Compound no. 129),
2-[(4′-Ethylbiphenyl-4-yl)sulfanyl]-4-(6-methoxy-4-oxo-1,2,3-benzotriazin-3(4H)-yl)butanoic acid (Compound no. 130),
2-[(3′,4′-Dimethylbiphenyl-4-yl)sulfanyl]-4-(6-methoxy-4-oxo-1,2,3-benzotriazin-3(4H)-yl)butanoic acid (Compound no. 131),
4-(6-Methoxy-4-oxo-1,2,3-benzotriazin-3(4H)-yl)-2-[(4′-methylbiphenyl-4-yl)sulfanyl]butanoic acid (Compound no. 132),
4-(6-Methoxy-4-oxo-1,2,3-benzotriazin-3(4H)-yl)-2-{[4′-(trifluoromethyl)biphenyl-4-yl]sulfanyl}butanoic acid (Compound no. 133),
2-[(4′-Ethylbiphenyl-4-yl)sulfanyl]-4-(8-methyl-4-oxo-1,2,3-benzotriazin-3(4H)-yl)butanoic acid (Compound no. 134),
2-[(3′-Fluoro-4′-methylbiphenyl-4-yl)sulfanyl]-4-(8-methyl-4-oxo-1,2,3-benzotriazin-3(4H)-yl)butanoic acid (Compound no. 135),
2-{[4-(6-Methoxypyridin-3-yl)phenyl]sulfanyl}-4-(8-methyl-4-oxo-1,2,3-benzotriazin-3(4H)-yl)butanoic acid (Compound no. 136),
2-[(4′-Methylbiphenyl-4-yl)sulfanyl]-4-(8-methyl-4-oxo-1,2,3-benzotriazin-3(4H)-yl)butanoic acid (Compound no. 137),
2-[(4′-Chlorobiphenyl-4-yl)sulfanyl]-4-(8-methyl-4-oxo-1,2,3-benzotriazin-3(4H)-yl)butanoic acid (Compound no. 138),
2-[(4′-Fluorobiphenyl-4-yl)sulfanyl]-4-(8-methyl-4-oxo-1,2,3-benzotriazin-3(4H)-yl)butanoic acid (Compound no. 139),
2-[(4′-Fluoro-3′-methylbiphenyl-4-yl)sulfanyl]-4-(8-methyl-4-oxo-1,2,3-benzotriazin-3(4H)-yl)butanoic acid (Compound no. 140),
2-[(3′,4′-Difluorobiphenyl-4-yl)sulfanyl]-4-(8-methyl-4-oxo-1,2,3-benzotriazin-3(4H)-yl)butanoic acid (Compound no. 141),
2-[(3′,4′-Dimethoxybiphenyl-4-yl)sulfanyl]-4-(8-methyl-4-oxo-1,2,3-benzotriazin-3(4H)-yl)butanoic acid (Compound no. 142),
2-[(3′,4′-Dimethylbiphenyl-4-yl)sulfanyl]-4-(8-methyl-4-oxo-1,2,3-benzotriazin-3(4H)-yl)butanoic acid (Compound no. 143),
4-(8-Methyl-4-oxo-1,2,3-benzotriazin-3(4H)-yl)-2-{[4′-(trifluoromethyl)biphenyl-4-yl]sulfanyl}butanoic acid (Compound no. 144),
2-[(3′-Fluoro-4′-methoxybiphenyl-4-yl)sulfanyl]-4-(8-methyl-4-oxo-1,2,3-benzotriazin-3(4H)-yl)butanoic acid (Compound no. 145),
4-(5-Chloro-4-oxo-1,2,33-benzotriazin-3(4H)-yl)-2-[(3′-fluoro-4′-methoxybiphenyl-4-yl)sulfanyl]butanoic acid (Compound no. 146),
4-(7-Chloro-4-oxo-1,2,3-benzotriazin-3(4H)-yl)-2-[(3′-fluoro-4′-methoxybiphenyl-4-yl)sulfanyl]butanoic acid (Compound no. 147),
2-{[4-(6-Methoxypyridin-3-yl)phenyl]sulfanyl}-4-(4-oxo-1,2,3-benzotriazin-3(4H)-yl)butanoic acid (Compound no. 148),
2-[(4′-Methoxybiphenyl-4-yl)sulfanyl]-4-(4-oxo-1,2,3-benzotriazin-3(4H)-yl)butanoic acid (Compound no. 149),
2-[(3′-Fluoro-4′-methylbiphenyl-4-yl)sulfanyl]-4-(4-oxo-1,2,3-benzotriazin-3(4H)-yl)butanoic acid (Compound no. 150),
2-[(3′,4′-Difluorobiphenyl-4-yl)sulfanyl]-4-(4-oxo-1,2,3-benzotriazin-3(4H)-yl)butanoic acid (Compound no. 151),
2-[(3′-Methoxybiphenyl-4-yl)sulfanyl]-4-(4-oxo-1,2,3-benzotriazin-3(4H)-yl)butanoic acid (Compound no. 152),
2-[(4′-Fluorobiphenyl-4-yl)sulfanyl]-4-(4-oxo-1,2,3-benzotriazin-3(4H)-yl)butanoic acid (Compound no. 153),
2-(Biphenyl-4-ylsulfanyl)-4-(4-oxo-1,2,3-benzotriazin-3(4H)-yl)butanoic acid (Compound no. 154),
2-[(2′,3′-Difluorobiphenyl-4-yl)sulfanyl]-4-(4-oxo-1,2,3-benzotriazin-3(4H)-yl)butanoic acid (Compound no. 155),
2-{[(4′-Chlorobiphenyl-4-yl)methyl]sulfanyl}-4-(4-oxo-1,2,3-benzotriazin-3(4H)-yl)butanoic acid (Compound no. 156),
2-{[(4′-Chlorobiphenyl-4-yl)methyl]sulfanyl}-4-(7-methyl-4-oxo-1,2,3-benzotriazin-3(4H)-yl)butanoic acid (Compound no. 157),
2-{[(4′-Chlorobiphenyl-4-yl)methyl]sulfanyl}-4-(7-methoxy-4-oxo-1,2,3-benzotriazin-3(4H)-yl)butanoic acid (Compound no. 158),
2-{[(4′-Chlorobiphenyl-4-yl)methyl]sulfanyl}-4-(6-fluoro-4-oxo-1,2,3-benzotriazin-3(4H)-yl)butanoic acid (Compound no. 159),
2-{[(4′-Chlorobiphenyl-4-yl)methyl]sulfanyl}-4-(8-methyl-4-oxo-1,2,3-benzotriazin-3(4H)-yl)butanoic acid (Compound no. 160),
2-{[(4′-Chlorobiphenyl-4-yl)methyl]sulfanyl}-4-[4-oxo-7-(trifluoromethyl)-1,2,3-benzotriazin-3(4H)-yl]butanoic acid (Compound no. 161),
2-{[(4′-Chlorobiphenyl-4-yl)methyl]sulfanyl}-4-(5-chloro-4-oxo-1,2,3-benzotriazin-3(4H)-yl)butanoic acid (Compound no. 162),
2-{[2-(4′-Chlorobiphenyl-4-yl)ethyl]sulfanyl}-4-(7-chloro-4-oxo-1,2,3-benzotriazin-3(4H)-yl)butanoic acid (Compound no. 163),
2-{[2-(4′-Chlorobiphenyl-4-yl)ethyl]sulfanyl}-4-(7-methyl-4-oxo-1,2,3-benzotriazin-3(4H)-yl)butanoic acid (Compound no. 164),
2-{[2-(4′-Chlorobiphenyl-4-yl)ethyl]sulfanyl}-4-(8-methyl-4-oxo-1,2,3-benzotriazin-3(4H)-yl)butanoic acid (Compound no. 165),
2-[(4′-tert-Butylbiphenyl-4-yl)sulfanyl]-4-(4-oxo-1,2,3-benzotriazin-3(4H)-yl)butanoic acid (Compound no. 166),
2-[(4′-Ethylbiphenyl-4-yl)sulfanyl]-4-(4-oxo-1,2,3-benzotriazin-3(4H)-yl)butanoic acid (Compound no. 167),
4-(4-Oxo-1,2,3-benzotriazin-3(4H)-yl)-2-{[4′-(propan-2-yl)biphenyl-4-yl]sulfanyl}butanoic acid (Compound no. 168),
4-(4-Oxo-1,2,3-benzotriazin-3(4H)-yl)-2-{[4′-(trifluoromethyl)biphenyl-4-yl]sulfanyl}butanoic acid (Compound no. 169),
4-(4-Oxo-1,2,3-benzotriazin-3(4H)-yl)-2-{[4′-(trifluoromethoxy)biphenyl-4-yl]sulfanyl}butanoic acid (Compound no. 170),
2-[(3′-Fluoro-4′-methoxybiphenyl-4-yl)sulfanyl]-4-(7-methoxy-4-oxo-1,2,3-benzotriazin-3(4H)-yl)butanoic acid (Compound no. 171),
4-(7-Methoxy-4-oxo-1,2,3-benzotriazin-3(4H)-yl)-2-{[4-(6-methoxypyridin-3-yl)phenyl]sulfanyl}butanoic acid (Compound no. 172),
4-(7-Methoxy-4-oxo-1,2,3-benzotriazin-3(4H)-yl)-2-{[4′-(trifluoromethyl)biphenyl-4-yl]sulfanyl}butanoic acid (Compound no. 173),
2-[(4′-Methoxybiphenyl-4-yl)sulfanyl]-4-(7-methoxy-4-oxo-1,2,3-benzotriazin-3(4H)-yl)butanoic acid (Compound no. 174),
2-[(3′-Fluoro-4′-methylbiphenyl-4-yl)sulfanyl]-4-(7-methoxy-4-oxo-1,2,3-benzotriazin-3(4H)-yl)butanoic acid (Compound no. 175),
4-(7-Methoxy-4-oxo-1,2,3-benzotriazin-3(4H)-yl)-2-{[4-(1-methyl-1H-pyrazol-4-yl)phenyl]sulfanyl}butanoic acid (Compound no. 176),
2-[(3′,4′-Dimethoxybiphenyl-4-yl)sulfanyl]-4-(7-methoxy-4-oxo-1,2,3-benzotriazin-3(4H)-yl)butanoic acid (Compound no. 177),
2-[(4′-Ethylbiphenyl-4-yl)sulfanyl]-4-(7-methoxy-4-oxo-1,2,3-benzotriazin-3(4H)-yl)butanoic acid (Compound no. 178),
2-[(4′-Fluoro-3′-methylbiphenyl-4-yl)sulfanyl]-4-(7-methoxy-4-oxo-1,2,3-benzotriazin-3(4H)-yl)butanoic acid (Compound no. 179),
2-[(4′-Chlorobiphenyl-4-yl)sulfanyl]-4-(7-methoxy-4-oxo-1,2,3-benzotriazin-3(4H)-yl)butanoic acid (Compound no. 180),
2-[(3′,4′-Dichlorobiphenyl-4-yl)sulfanyl]-4-(7-methoxy-4-oxo-1,2,3-benzotriazin-3(4H)-yl)butanoic acid (Compound no. 181),
2-[(4-{[(4-Chlorophenyl)carbonyl]amino}phenyl)sulfanyl]-4-(4-oxo-1,2,3-benzotriazin-3(4H)-yl)butanoic acid (Compound no. 182),
2-[(4-{[(3-Methoxyphenyl)acetyl]amino}phenyl)sulfonyl]-4-(4-oxo-1,2,3-benzotriazin-3(4H)-yl)butanoic acid (Compound no. 183),
2-[(4-{[(2,5-Dimethoxyphenyl)acetyl]amino}phenyl)sulfonyl]-4-(4-oxo-1,2,3-benzotriazin-3(4H)-yl)butanoic acid (Compound no. 184),
4-(4-Oxo-1,2,3-benzotriazin-3(4H)-yl)-2-({4-(phenylacetyl)amino]phenyl}sulfonyl)butanoic acid (Compound no. 185),
2-(4-Fluorobenzyl)-2-[(2-{4-[(4-fluorobenzyl)oxy]phenyl}ethyl)sulfonyl]-4-(4-oxo-1,2,3-benzotriazin-3(4H)-yl)butanoic acid (Compound no. 186),
4-(6-Methoxy-4-oxo-1,2,3-benzotriazin-3(4H)-yl)-2-[(4-{[(4-methylphenyl)carbonyl]amino}phenyl)sulfanyl]butanoic acid (Compound no. 187),
4-(8-Methyl-4-oxo-1,2,3-benzotriazin-3(4H)-yl)-2-[(4-{[(4-methylphenyl)carbonyl]amino}phenyl)sulfanyl]butanoic acid (Compound no. 188),
4-(6-Methoxy-4-oxo-1,2,3-benzotriazin-3(4H)-yl)-2-[(4-{[(4-methylphenyl)carbonyl]amino}phenyl)sulfonyl]butanoic acid (Compound no. 189),
4-(8-Methyl-4-oxo-1,2,3-benzotriazin-3(4H)-yl)-2-[(4-{[(4-methylphenyl)carbonyl]amino}phenyl)sulfonyl]butanoic acid (Compound no. 190),
2-(3-Fluorobenzyl)-2-[(2-{4-[(3-fluorobenzyl)oxy]phenyl}ethyl)sulfonyl]-4-(4-oxo-1,2,3-benzotriazin-3(4H)-yl)butanoic acid (Compound no. 191),
4-(6-Methyl-4-oxo-1,2,3-benzotriazin-3(4H)-yl)-2-[(4-{[(4-methylphenyl)carbonyl]amino}phenyl)sulfanyl]butanoic acid (Compound no. 192),
4-(7-Methyl-4-oxo-1,2,3-benzotriazin-3(4H)-yl)-2-[(4-{[(4-methylphenyl)carbonyl]amino}phenyl)sulfanyl]butanoic acid (Compound no. 193),
4-(6-Fluoro-4-oxo-1,2,3-benzotriazin-3(4H)-yl)-2-[(4-{[(4-methylphenyl)carbonyl]amino}phenyl)sulfanyl]butanoic acid (Compound no. 194),
2-{[2-(4′-Chlorobiphenyl-4-yl)ethyl]sulfanyl}-4-(6-fluoro-4-oxo-1,2,3-benzotriazin-3(4H)-yl)butanoic acid (Compound no. 195),
2-{[2-(4′-Chlorobiphenyl-4-yl)ethyl]sulfanyl}-4-(6-methyl-4-oxo-1,2,3-benzotriazin-3(4H)-yl)butanoic acid (Compound no. 196),
2-{[2-(4′-Chlorobiphenyl-4-yl)ethyl]sulfonyl}-4-(7-methyl-4-oxo-1,2,3-benzotriazin-3(4H)-yl)butanoic acid (Compound no. 197),
2-(2-Chlorobenzyl)-2-[(2-{4-[(2-chlorobenzyl)oxy]phenyl}ethyl)sulfonyl]-4-(4-oxo-1,2,3-benzotriazin-3(4H)-yl)butanoic acid (Compound no. 198),
2-{[2-(4′-Chlorobiphenyl-4-yl)ethyl]sulfonyl}-4-(6-methyl-4-oxo-1,2,3-benzotriazin-3(4H)-yl)butanoic acid (Compound no. 199),
4-(6-Methyl-4-oxo-1,2,3-benzotriazin-3(4H)-yl)-2-[(4-{[(4-methylphenyl)carbonyl]amino}phenyl)sulfonyl]butanoic acid (Compound no. 200),
4-(7-Methyl-4-oxo-1,2,3-benzotriazin-3(4H)-yl)-2-[(4-{[(4-methylphenyl)carbonyl]amino}phenyl)sulfonyl]butanoic acid (Compound no. 201),
2-[(4-{[(4-Methoxyphenyl)carbonyl]amino}phenyl)sulfanyl]-4-(6-methyl-4-oxo-1,2,3-benzotriazin-3(4H)-yl)butanoic acid (Compound no. 202),
2-[(4-{[(4-Fluorophenyl)carbonyl]amino}phenyl)sulfanyl]-4-(6-methyl-4-oxo-1,2,3-benzotriazin-3(4H)-yl)butanoic acid (Compound no. 203),
2-[(4-{[(3,4-Dichlorophenyl)carbonyl]amino}phenyl)sulfanyl]-4-(6-methyl-4-oxo-1,2,3-benzotriazin-3(4H)-yl)butanoic acid (Compound no. 204),
2-[(4-{[(4-Ethylphenyl)carbonyl]amino}phenyl)sulfanyl]-4-(6-methyl-4-oxo-1,2,3-benzotriazin-3(4H)-yl)butanoic acid (Compound no. 205),
2-[(4-{[(4-Methoxyphenyl)carbonyl]amino}phenyl)sulfonyl]-4-(6-methyl-4-oxo-1,2,3-benzotriazin-3(4H)-yl)butanoic acid (Compound no. 206),
2-[(4-{[(3,4-Dichlorophenyl)carbonyl]amino}phenyl)sulfonyl]-4-(6-methyl-4-oxo-1,2,3-benzotriazin-3(4H)-yl)butanoic acid (Compound no. 207),
2-[(4-{[(4-Ethylphenyl)carbonyl]amino}phenyl)sulfonyl]-4-(6-methyl-4-oxo-1,2,3-benzotriazin-3(4H)-yl)butanoic acid (Compound no. 208),
2-[(4-{[(4-Fluorophenyl)carbonyl]amino}phenyl)sulfonyl]-4-(6-methyl-4-oxo-1,2,3-benzotriazin-3(4H)-yl)butanoic acid (Compound no. 209),
2-[(4-{[(4-Chlorophenyl)carbonyl]amino}phenyl)sulfonyl]-4-(6-methyl-4-oxo-1,2,3-benzotriazin-3(4H)-yl)butanoic acid (Compound no. 210),
2-[(4′-Chlorobiphenyl-4-yl)oxy]-4-(4-oxo-1,2,3-benzotriazin-3(4H)-yl)butanoic acid (Compound no. 211),
2-[(4′-Chlorobiphenyl-4-yl)methoxy]-4-(6-fluoro-4-oxo-1,2,3-benzotriazin-3(4H)-yl)butanoic acid (Compound no. 212),
2-[(4′-Chlorobiphenyl-4-yl)oxy]-4-(7-chloro-4-oxo-1,2,3-benzotriazin-3(4H)-yl)butanoic acid (Compound no. 213),
2-[(4′-Chlorobiphenyl-4-yl)oxy]-4-[4-oxo-7-(trifluoromethyl)-1,2,3-benzotriazin-3(4H)-yl]butanoic acid (Compound no. 214),
2-[(4′-Chlorobiphenyl-4-yl)oxy]-4-(5-fluoro-4-oxo-1,2,3-benzotriazin-3(4H)-yl)butanoic acid (Compound no. 215),
2-[(4′-Chlorobiphenyl-4-yl)oxy]-4-(5-chloro-4-oxo-1,2,3-benzotriazin-3(4H)-yl)butanoic acid (Compound no. 216),
2-[(4′-Chlorobiphenyl-4-yl)oxy]-4-(6-methoxy-4-oxo-1,2,3-benzotriazin-3(4H)-yl)butanoic acid (Compound no. 217),
2-[(4′-Chlorobiphenyl-4-yl)oxy]-4-(5-methyl-4-oxo-1,2,3-benzotriazin-3(4H)-yl)butanoic acid (Compound no. 218),
(2R)-2-[(4′-Chlorobiphenyl-4-yl)oxy]-4-(6-fluoro-4-oxo-1,2,3-benzotriazin-3(4H)-yl)butanoic acid (Compound no. 219),
(2R)-2-[(4′-Chlorobiphenyl-4-yl)oxy]-4-(7-chloro-4-oxo-1,2,3-benzotriazin-3(4H)-yl)butanoic acid (Compound no. 220),
(2R)-2-[(4′-Chlorobiphenyl-4-yl)oxy]-4-(6-methoxy-4-oxo-1,2,3-benzotriazin-3(4H)-yl)butanoic acid (Compound no. 221),
(2R)-2-[(4′-Chlorobiphenyl-4-yl)oxy]-4-(6,7-difluoro-4-oxo-1,2,3-benzotriazin-3(4H)-yl)butanoic acid (Compound no. 222),
2-[(4′-Chlorobiphenyl-4-yl)methoxy]-4-(7-chloro-4-oxo-1,2,3-benzotriazin-3(4H)-yl)butanoic acid (Compound no. 223),
2-[(4′-Chlorobiphenyl-4-yl)methoxy]-4-(7-methoxy-4-oxo-1,2,3-benzotriazin-3(4H)-yl)butanoic acid (Compound no. 224),
(2R)-2-[(4′-Chlorobiphenyl-4-yl)oxy]-4-(2,4-dioxo-2H-1,3-benzoxazin-3(4H)-yl)butanoic acid (Compound no. 225),
(2R)-2-[(4′-Chlorobiphenyl-4-yl)oxy]-4-(1-oxophthalazin-2(1H)-yl)butanoic acid (Compound no. 226),
2-[(4′-Chlorobiphenyl-4-yl)methoxy]-4-(4-oxo-1,2,3-benzotriazin-3(4H)-yl)butanoic acid (Compound no. 227),
2-[(4′-Chlorobiphenyl-4-yl)methoxy]-4-(1-oxophthalazin-2(1H)-yl)butanoic acid (Compound no. 228),
2-[(4′-Chlorobiphenyl-4-yl)methoxy]-4-(2,4-dioxo-2H-1,3-benzoxazin-3(4H)-yl)butanoic acid (Compound no. 229).
including racemates, enantiomers and diastereomers thereof, or a pharmaceutically acceptable salt.

4. A pharmaceutical composition comprising a therapeutically effective amount of a compound according to any one of claims 1 to 3, together with a pharmaceutically acceptable carrier, excipient or diluents.

5. A compound according to any one of claims 1 to 3, for use in the treatment or prophylaxis of an animal or a human suffering from an inflammatory or allergic disease.

6. A compound according to claim 5, wherein the inflammatory disease or allergic disease is asthma, rheumatoid arthritis, COPD, dry eye, rhinitis, osteoarthritis, psoriatic arthritis, psoriasis, pulmonary fibrosis, pulmonary inflammation, acute respiratory distress syndrome, periodontitis, multiple sclerosis, gingivitis, atherosclerosis, dry eye, neointimal proliferation associated with restenosis and ischemic heart failure, stroke, renal disease or tumor metastasis.

7. A pharmaceutical composition according to claim 4, further comprising one or more additional active ingredients selected from:
a) Anti-inflammatory agents selected from (i) nonsteroidal anti-inflammatory agents piroxicam, diclofenac, propionic acids, fenamates, pyrazolones, salicylates, PDE-4/p38 MAP Kinase/Cathepsin inhibitors, CCR-3 antagonists, iNOS inhibitors, tryptase and elastase inhibitors, beta-2 integrin antagonists, cell adhesion inhibitors (especially ICAM), adenosine 2a agonists; (ii) leukotrienes LTC4/LTD4/LTE4/LTB4-Inhibitors, 5-lipoxygenase inhibitors and PAF-receptor antagonists; (iii) Cox-2 inhibitors; (iv) other MMP inhibitors; (v) interleukin-I inhibitors; and (vi) corticosteroids, such as alclometasone, amcinonide, amelometasone, beclometasone, betamethasone, budesonide, ciclesonide, clobetasol, cloticasone, cyclomethasone, deflazacort, deprodone, dexbudesonide, diflorasone, difluprednate, fluticasone, flunisolide, halometasone, haloperedone, hydrocortisone, methylprednisolone, mometasone, prednicarbate, prednisolone, rimexolone, tixocortol, triamcinolone, ulobetasol, rofleponide, GW 215864, KSR 592, ST-126, dexamethasone and pharmaceutically acceptable salts, solvates thereof. Preferred corticosteroids include, for example, flunisolide, beclomethasone, triamcinolone, budesonide, fluticasone, mometasone, ciclesonide, and dexamethasone;
b) beta-agonists, selected from one or more β2-agonists—albuterol, salbutamol, biltolterol, pirbuterol, levosalbutamol, tulobuterol, terbutaline, bambuterol, metaproterenol, fenoterol, salmeterol, carmoterol, arformoterol, formoterol, and their pharmaceutically acceptable salts, or solvates thereof;
c) antihypertensive agents, selected from (i) the ACE inhibitors, enalapril, lisinopril, valsartan, telmisartan and quinapril; (ii) angiotensin II receptor antagonists and agonists, e.g., losartan, candesartan, irbesartan, valsartan, and eprosartan; (iii) β-blockers; and (iv) calcium channel blockers;
d) immunosuppressive agents, selected from cyclosporine, azathioprine and methotrexate, anti-inflammatory corticosteroids; and
e) anti-infective agents.

8. A process for preparing a compound of Formula X [Formula I when R is H, X¹ is G (O and S) U—V—W— is a bond and A is phenyl] 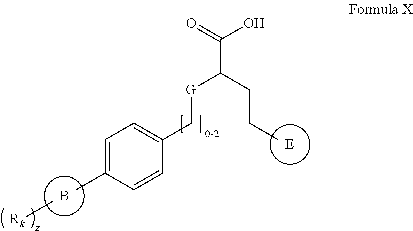
comprising:
a) converting a compound of Formula II 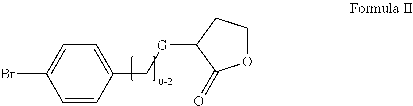
to give a compound of Formula III; 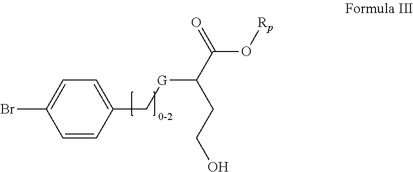
b) reacting a compound of Formula III with a compound of Formula IV to give a compound of Formula V; 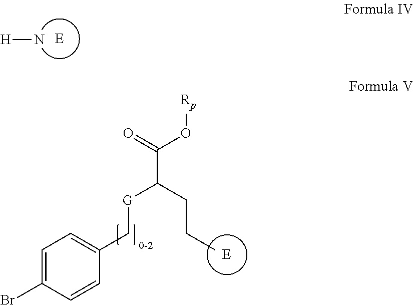
coupling a compound of Formula V with a compound of Formula VI to give a compound of Formula VII 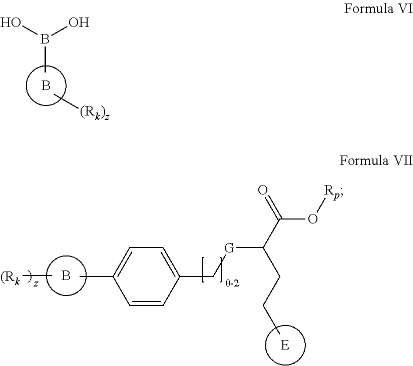
c) deprotecting a compound of Formula VII to give a compound of Formula X;
or
d) coupling a compound of Formula II with a compound of Formula VI 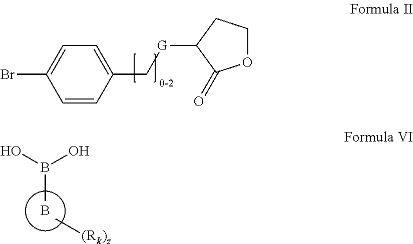
to give a compound of Formula VIII; 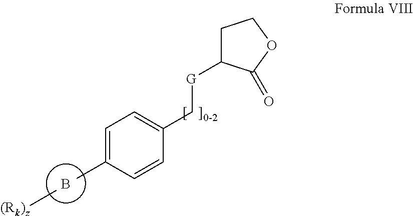
e) converting a compound of Formula VIII to give a compound of Formula IX; 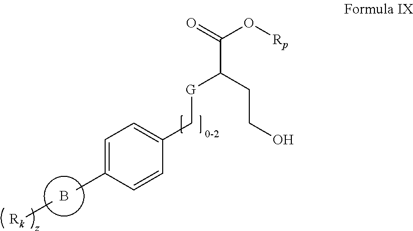
f) reacting a compound of Formula IX with a compound of Formula IV to give a compound of Formula VII; 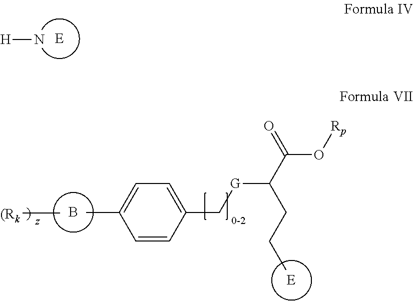
g) deprotecting a compound of Formula VII to give a compound of Formula X wherein,
G is O or S;
Rₚ is a carboxy protecting group such as methyl, ethyl, allyl, benzyl, t-butyl and silyl; 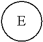
represents mono-, bi- or polycyclic heteroaryl or heterocyclyl selected from the following 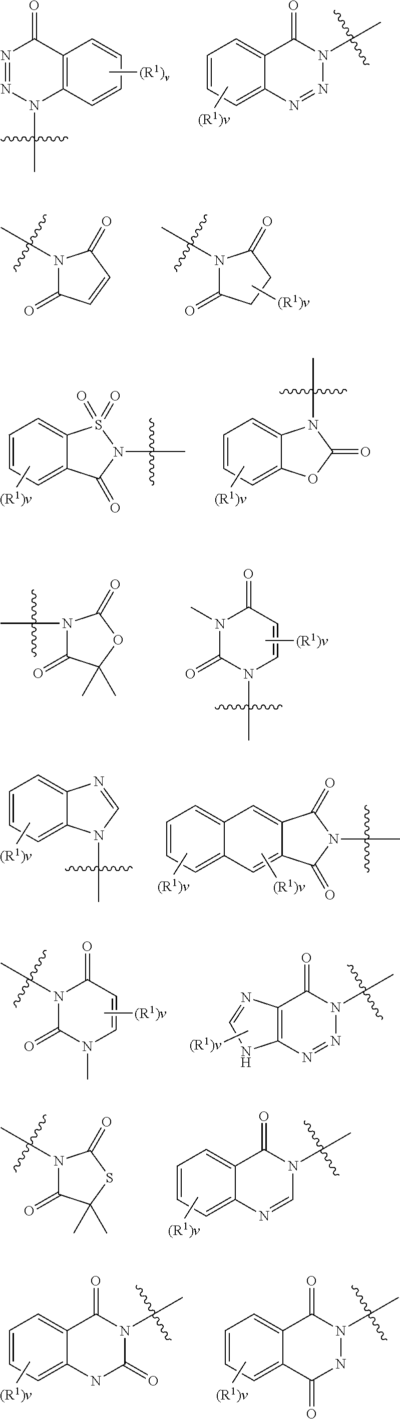 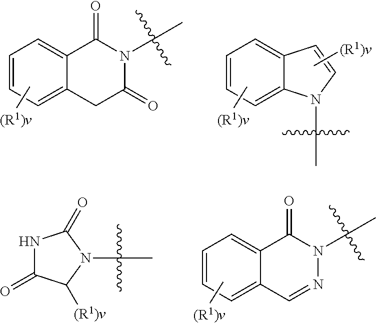 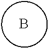
represents C₆-C₁₂ aryl, C₃-C₁₂ cycloalkyl, C₆-C₁₂ heteroaryl or C₆-C₁₂ heterocyclyl, each of which is optionally further substituted by one or more substituents independently selected from R¹;
R¹ represents alkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙ NHC(═O)NR_{f}R_{q}, —(CH₂)ₙ—O—C(═O)—R_{f}, —(CH₂)ₙ—NH—C(═O)—R_{f} or —(CH₂)ₙS(—O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl};
n is zero or an integer between 1 and 2;
m is an integer from 0-2;
z is an integer from 0-2; and
Rₖ is H, halo, alkyl, alkoxy, cyano, halogeno-C₁-C₆ alkyl, halogeno-C₁-C₆ alkoxy.

9. A process for preparing a compound of Formula XI [Formula I wherein R is H, X¹ is SO₂, U—V—W— is a bond and A is phenyl] 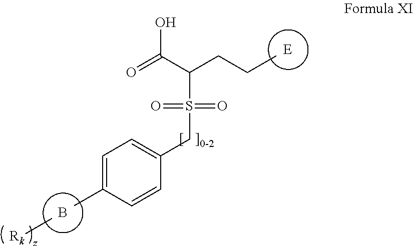
comprising:
a) oxidizing a compound of Formula X (when G is S) 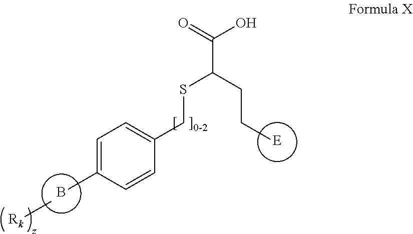
to give a compound of Formula XI;
wherein, 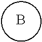
represents C₆-C₁₂ aryl, C₃-C₁₂ cycloalkyl, C₆-C₁₂ heteroaryl or C₆-C₁₂ heterocyclyl, each of which is optionally further substituted by one or more substituents independently selected from R¹;
R¹ represents alkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙ NHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q}{wherein R_{f} and R_{q} each independently represent hydrogen, alkyl, alkenyl, cycloalkyl aryl, heteroaryl, heterocyclyl, alkylaryl, alkylheteroaryl and alkylheterocyclyl};
n is zero or an integer between 1 and 2;
m is an integer 0-2; 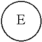
represents mono, bi or polycyclic heteroaryl or heterocyclyl selected from the following 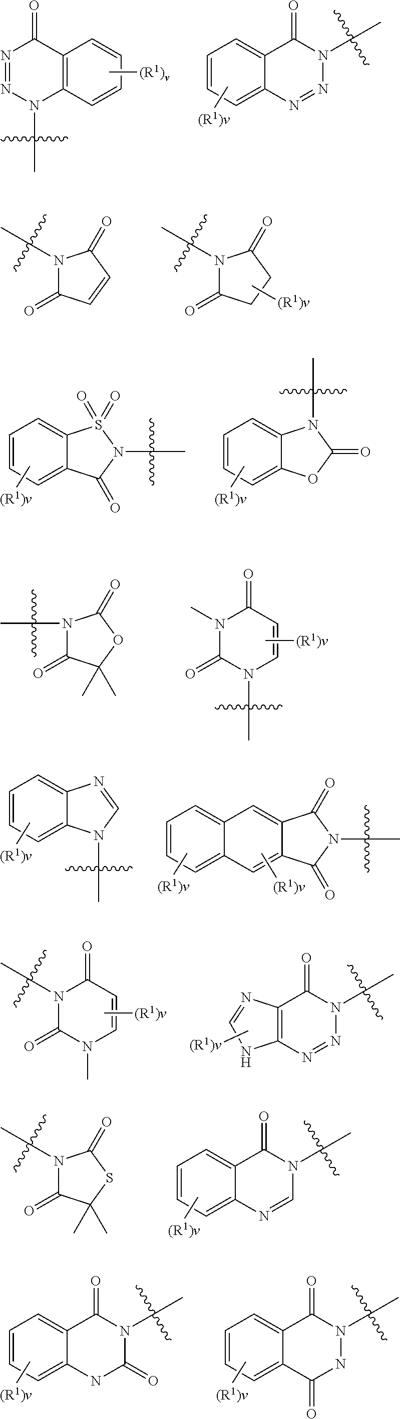 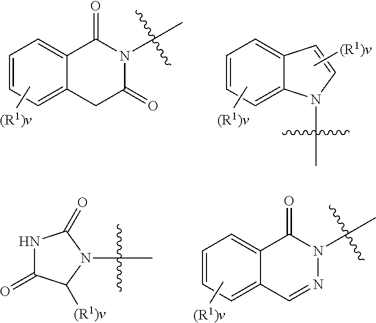
z is an integer 0-2; and
Rₖ is H, halo, alkyl, alkoxy, cyano, halogeno-C₁-C₆ alkyl, halogeno-C₁-C₆ alkoxy.

10. A process for preparing a compound of Formula XXVI [Formula I wherein R is H, X¹ is SO₂, U—V—W— is a bond and A is phenyl] 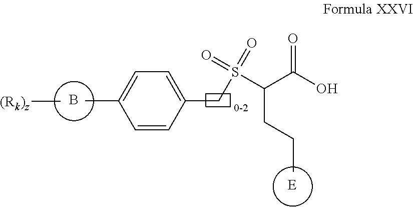
comprising:
a) oxidizing a compound of Formula XIV (Rₘ is Br or NO₂) to give a compound of Formula XV; 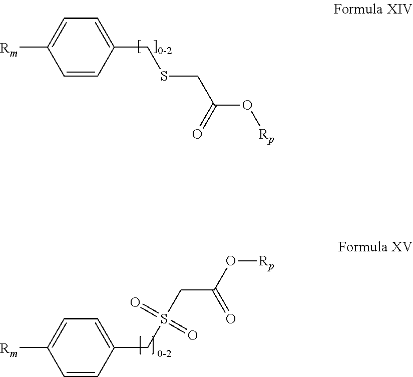
b) reacting a compound of Formula XV with a compound of Formula XVI to give a compound of Formula XVII; 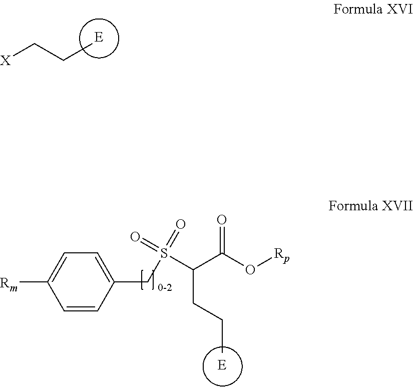
c) coupling a compound of Formula XVII (wherein Rₘ is Br) with a compound of Formula VI to give a compound of Formula XXV; 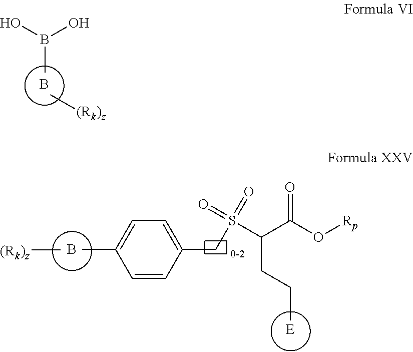
d) hydrolyzing a compound of Formula XXV to give a compound of Formula XXVI;
wherein,
Rₚ is carboxy protecting group such as methyl, ethyl, allyl, benzyl, t-butyl and silyl;
X is a leaving group such as halogen, mesylate, triflate; 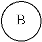
represents C₆-C₁₂ aryl, C₃-C₁₂ cycloalkyl, C₆-C₁₂ heteroaryl or C₆-C₁₂ heterocyclyl, each of which is optionally further substituted by one or more substituents independently selected from R¹;
R¹ represents salkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙNHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl};
n is zero or an integer between 1 and 2; 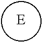
represents mono-, bi- or polycyclic heteroaryl or heterocyclyl selected 36 from the following: 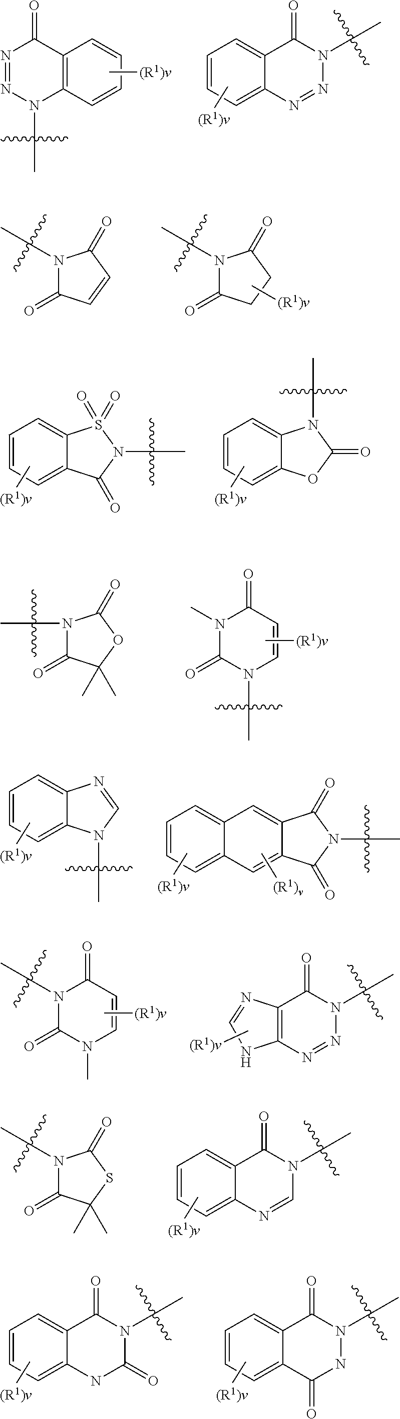 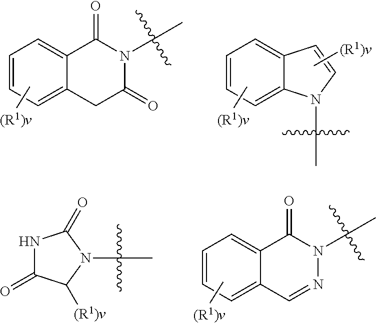
m is an integer 0-2;
z is an integer 0-2; and
Rₖ is H, halo, alkyl, alkoxy, cyano, halogeno-C₁-C₆ alkyl, halogeno-C₁-C₆ alkoxy.

11. A process for preparing a compound of Formula XXIX [Formula I wherein R is H, X¹ is SO₂, U is —NH—, V is —CO—, W is —NH— and A is phenyl] and Formula XXXII [Formula I wherein R is H, X¹ is SO₂, W is —NH—, V—U is —Rj-(-(CH₂)₀₋₁—CO—, —C(O)O—, —SO₂—) and A is phenyl] comprising:
a) O-protecting a compound of Formula XIX to give a compound of Formula XX; 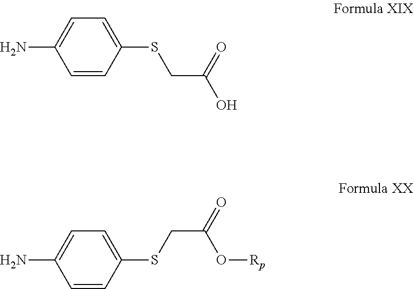
b) N-protecting a compound of Formula XX to give a compound of Formula XXI; 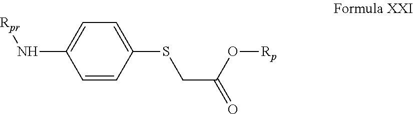
c) oxidizing a compound of Formula XXI to give a compound of Formula XXII; 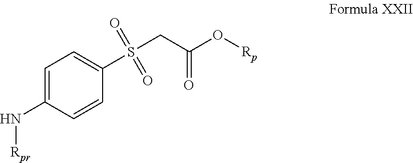
d) reacting a compound of Formula XXII with a compound of Formula XVI to 18 give a compound of Formula XXIII; 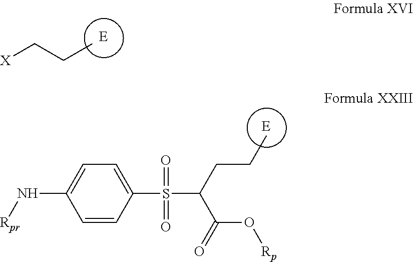
e) deprotecting a compound of Formula XXIII to give a compound of Formula XXIV; 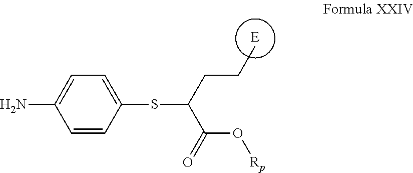
or
f) reducing a compound of Formula XVII (wherein Rₘ is NO₂) to give a compound of Formula XXIV; 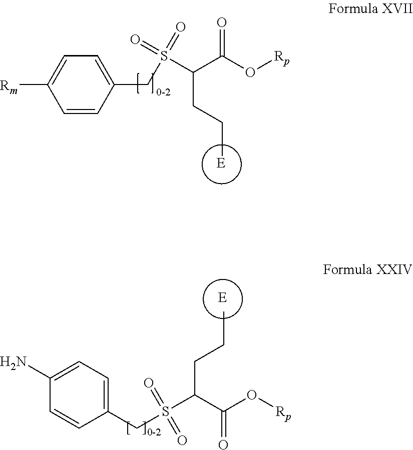
g) coupling of a compound of Formula XXIV with a compound of Formula XXVII to give a compound of Formula XXVIII; 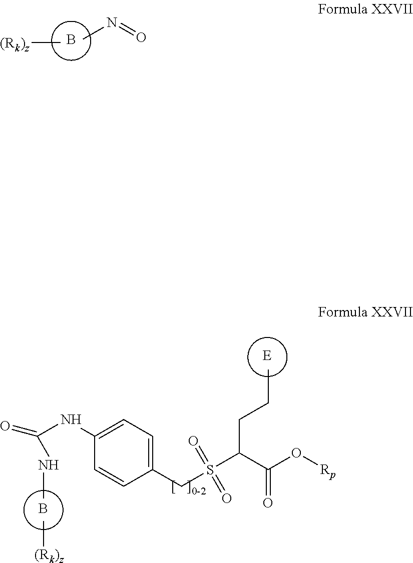
h) hydrolyzing a compound of Formula XXVIII to give a compound of Formula XXIX; 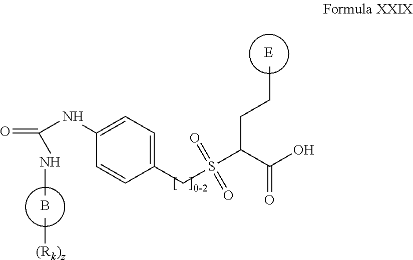
or
i) coupling of a compound of Formula XXIV with a compound of Formula XXX 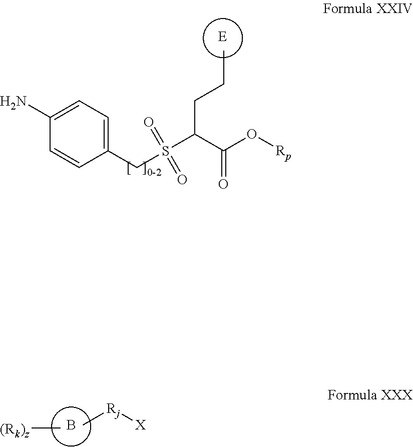
to give a compound of Formula XXXI; 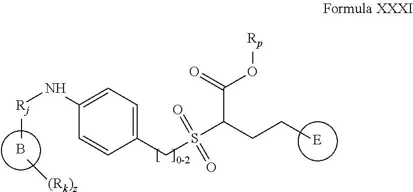
j) hydrolyzing a compound of Formula XXXI to give a compound of Formula XXXII; 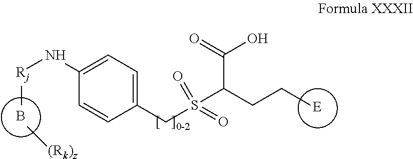
wherein,
Rₚ is carboxy protecting group such as methyl, ethyl, allyl, benzyl, t-butyl and silyl;
Rₚᵣ is an amino protecting group selected from di-tert-butyl dicarbonate, t-Boc, F-moc, benzyl, tosyl or carbobenzyloxy; 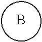
represents C₆-C₁₂ aryl, C₃-C₁₂ cycloalkyl, C₆-C₁₂ heteroaryl or C₆-C₁₂ heterocyclyl, each of which can be further substituted by one or more substituents independently selected from R¹;
R¹ represents salkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙNHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl};
n is zero or an integer between 1 and 2;
m is an integer 0-2;
X is a leaving group such as halogen, mesylate, triflate; 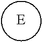
represents mono-, bi- or polycyclic heteroaryl or heterocyclyl selected from the following: 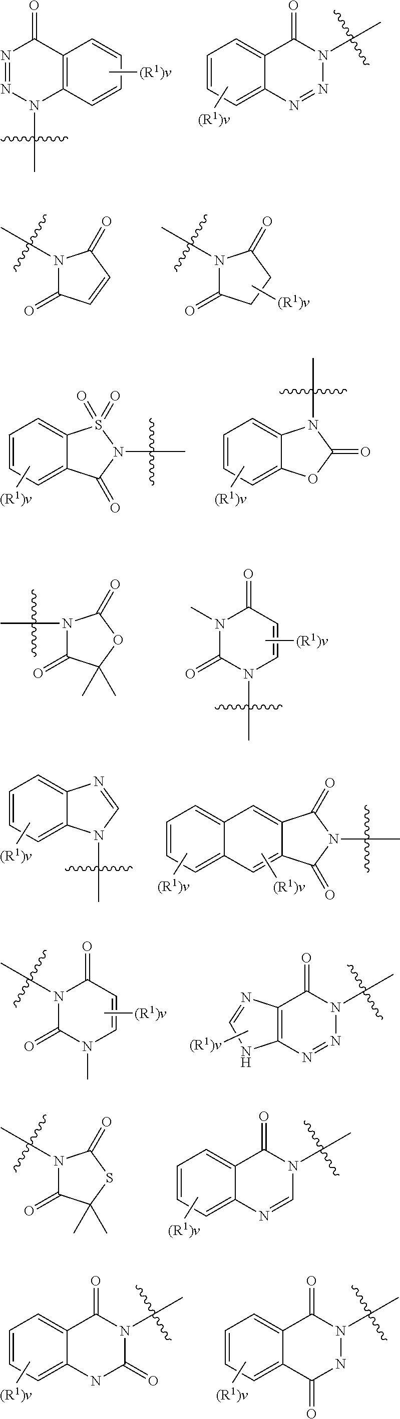 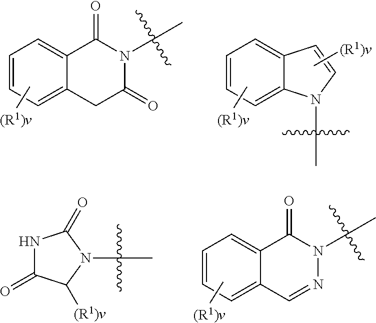
Rₖ is H, halo, alkyl, alkoxy, cyano, halogeno-C₁-C₆ alkyl, halogeno-C₁-C₆ alkoxy; and
z is an integer 0-2.

12. A process for preparing a compound of Formula XLI [Formula I wherein R is H, X¹ is S, W is —NH—, V—U is —Rj-(—(CH₂)₀₋₁—CO—, —C(O)O—, —SO₂—) and A is phenyl] 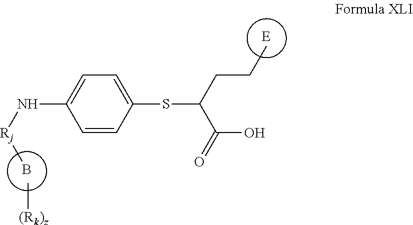
comprising
a) reducing a compound of Formula XXXIII to give a compound of Formula XXXIV; 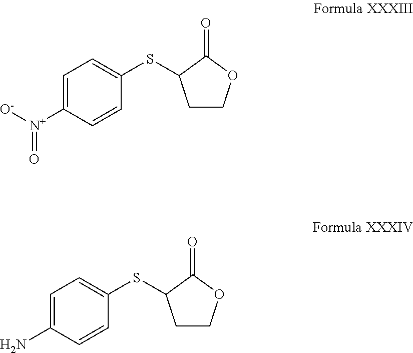 [SMILES: O=C1OCCC1Sc1ccc([N+](=O)[O-])cc1 ; Nc1ccc(SC2CCOC2=O)cc1]
b) reacting a compound of Formula XXXIV with a compound of Formula XXX to give a compound of Formula XXXV 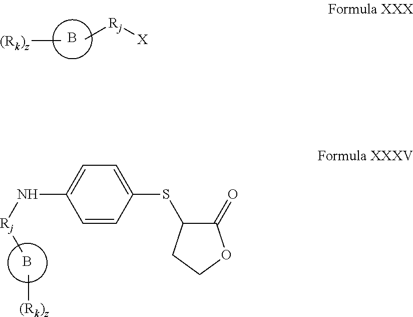
c) converting a compound of Formula XXXV to a compound of Formula XXXVI; 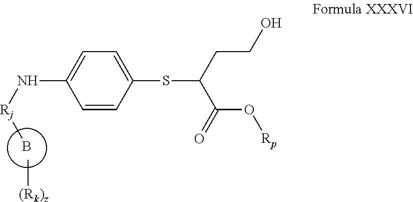
d) reacting a compound of Formula XXXVI with a compound of Formula IV to give a compound of Formula XXXVII; 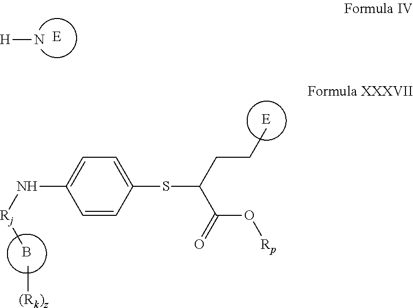
e) hydrolyzing a compound of Formula XXXVII to give a compound of Formula XLI;
or
f) converting a compound of Formula XXXIII, to a compound of Formula XXXVIII; 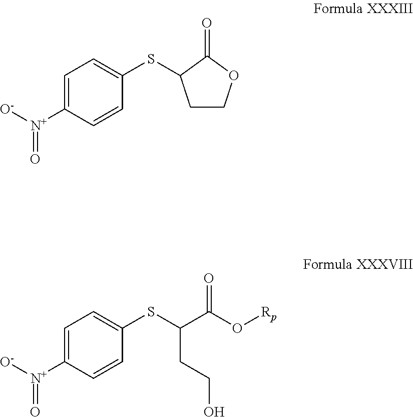
g) coupling a compound of Formula XXXVIII with a compound of Formula IV to give a compound of Formula XXXIX; 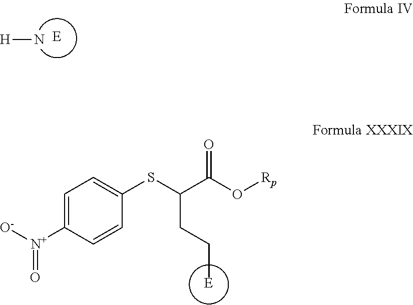
h) reducing a compound of Formula XXXIX to give a compound of Formula XL; 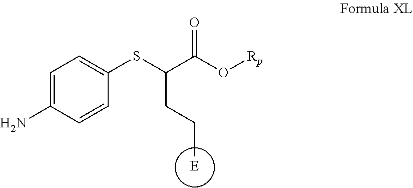
i) reacting a compound of Formula XL with a compound of Formula XXX to give a compound of Formula XXXVII; 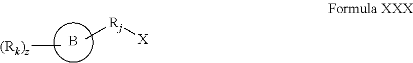
j) deprotecting a compound of Formula XXXVII to give a compound of Formula XLI;
wherein,
Rₚ is a carboxy protecting group such as methyl, ethyl, allyl, benzyl, t-butyl and silyl; 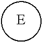
represents mono, bi or polycyclic heteroaryl or heterocyclyl selected from the following: 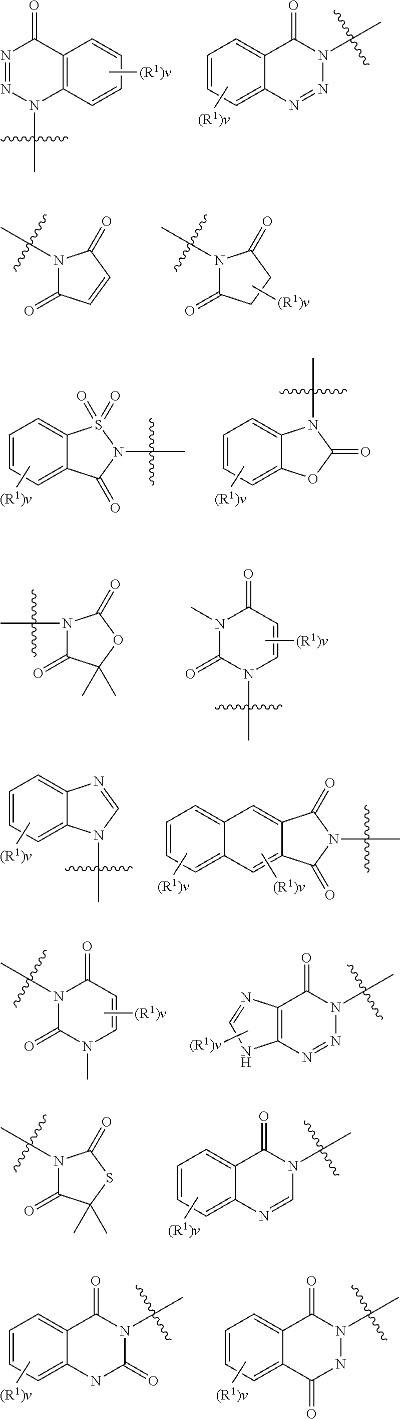 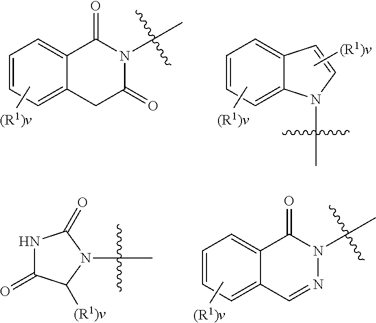
represents C₆-C₁₂ aryl, C₃-C₁₂ cycloalkyl, C₆-C₁₂ heteroaryl or C₆-C₁₂ heterocyclyl, each of which is optionally further substituted by one or more substituents independently selected from R¹;
R¹ represents alkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙNHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl};
n is zero or an integer between 1 and 2;
m is an integer 0-2;
X is a leaving group such as halogen, mesylate, triflate;
Rⱼ is (—(CH₂)₀₋₁—CO—, —C(O)O—, —SO₂—;
Rₖ is H, halo, alkyl, alkoxy, cyano, halogeno-C₁-C₆ alkyl, halogeno-C₁-C₆ alkoxy; and
z is an integer 0-2.

13. A process for preparing a compound of Formula XXXII [Formula I wherein R is H, X¹ is SO₂, W is —NH—, V—U is —Rj-(-(CH₂)₀₋₁—CO—, —C(O)O—, —SO₂) and A is phenyl] 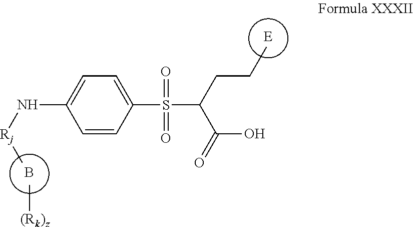
comprising:
a) oxidizing a compound of Formula XLI 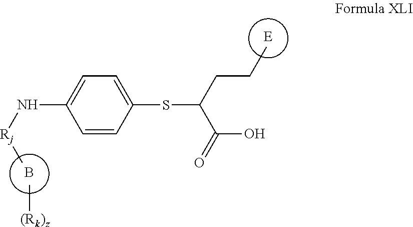
to give a compound of Formula XXXII;
wherein, 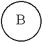
represents C₆-C₁₂ aryl, C₃-C₁₂ cycloalkyl, C₆-C₁₂ heteroaryl or C₆-C₁₂ heterocyclyl, each of which is optionally further substituted by one or more substituents independently selected from R¹;
R¹ represents alkyl, alkenyl, alkynyl, cyano, nitro, halogen, halogeno-C₁-C₆ alkyl, halogeno-C₁-C₆ alkoxy, azido, thiol, alkylthiol, —(CH₂)ₙ—OR_{f}, —C(═O)—R_{f}, —COOR_{f}, —NR_{f}R_{q}, —(CH₂)ₙ—C(═O)NR_{f}R_{q}, —(CH₂)ₙ—NHC(═O)—R_{f}, —(CH₂)ₙ—O—C(═O)—NR_{f}R_{q}, (CH₂)ₙNHC(═O)NR_{f}R_{q}, —(CH₂)ₙ—O—C(═O)—R_{f}, —(CH₂)ₙ—NH—C(═O)—R_{f} or —(CH₂)ₙS(═O)ₘ—NR_{f}R_{q} {wherein R_{f} and R_{q} each independently represent hydrogen, alkyl, alkenyl, cycloalkyl aryl, heteroaryl, heterocyclyl, alkylaryl, alkylheteroaryl and alkylheterocyclyl};
n is zero or an integer between 1 and 2;
m is an integer 0-2;
Rⱼ is (—(CH₂)₀₋₁—CO—, —C(O)O—, —SO₂—;
Rₖ is H, halo, alkyl, alkoxy, cyano, halogeno-C₁-C₆ alkyl, halogeno-C₁-C₆ alkoxy; and
z is an integer 0-2; 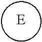
represents mono, bi or polycyclic heteroaryl or heterocyclyl selected from the following:

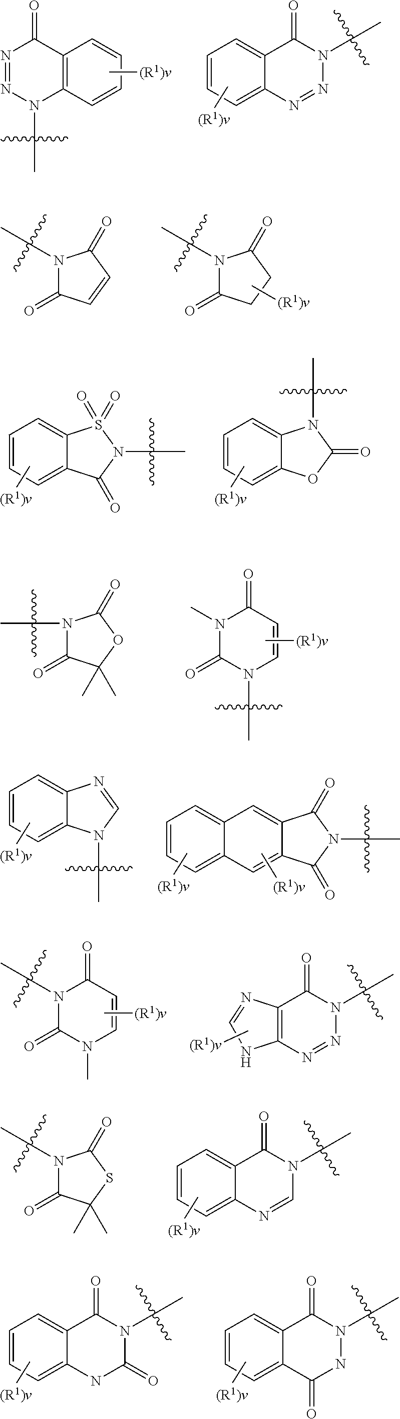

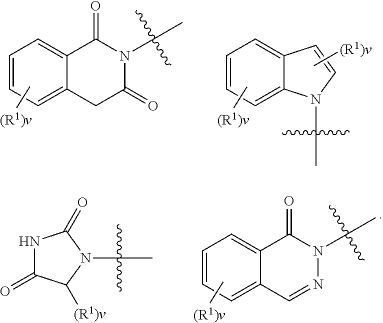

14. A process for preparing a compound of Formula XLV [Formula I wherein R is H, X¹ is SO₂, U is —CH₂—, V is -bond, W is —O—, A and B are phenyl] 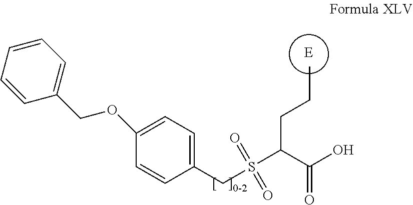
comprising:
a) oxidizing a compound of Formula XLII to give a compound of Formula XLIII; 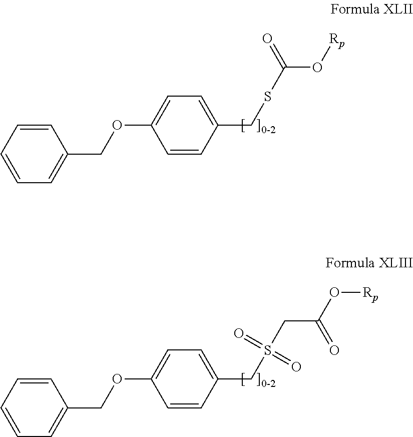
b) reacting a compound of Formula XLIII with a compound of Formula XVI to give a compound of Formula XLIV; 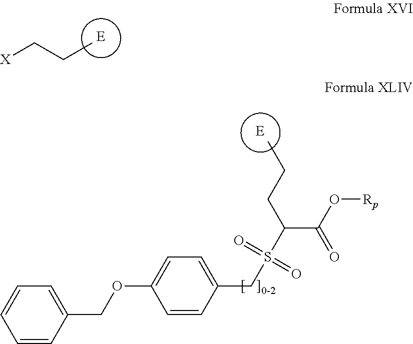
c) hydrolyzing a compound of Formula XLIV to give a compound of Formula XLV;
wherein,
Rₚ is a carboxy protecting group such as methyl, ethyl, allyl, benzyl, t-butyl and silyl;
X is a leaving group such as halogen, mesylate, triflate; and 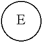
is selected from mono, bi or polycyclic heteroaryl or heterocyclyl selected from the following:

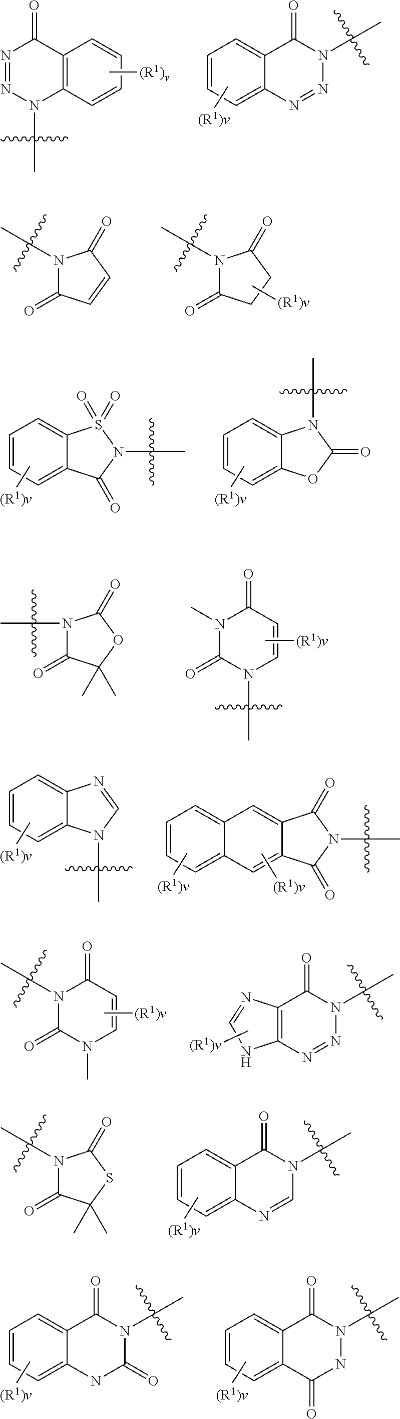

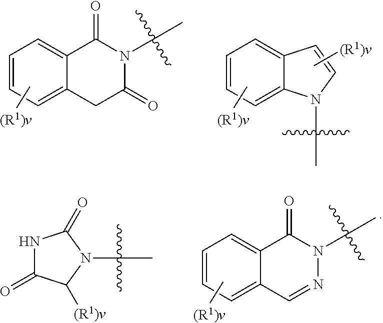

15. A process for preparing a compound of Formula XLVIII [Formula I wherein R is arylalkyl (benzyl), X¹ is SO₂, U is —CH₂—, V is -bond, W is —O— and A and B are phenyl] comprising: 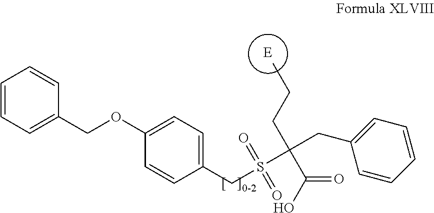
a) deprotecting a compound of Formula XLIV to give a compound of Formula XLVI; 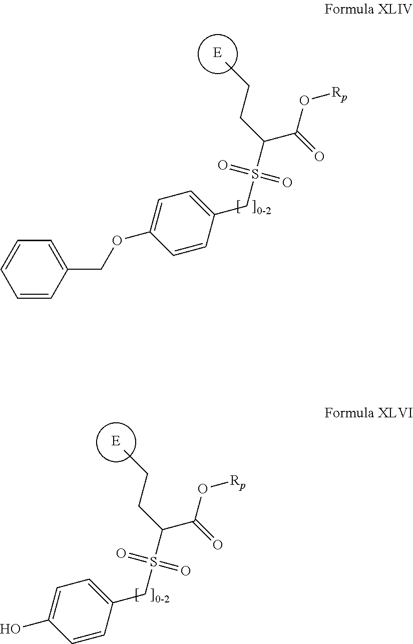
b) reacting a compound of Formula XLVI to give a compound of Formula XLVII; 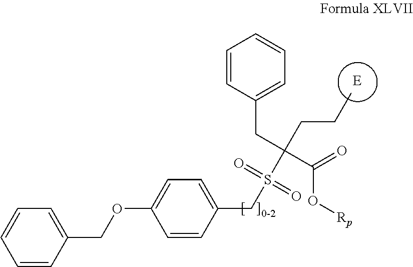
c) hydrolyzing a compound of Formula XLVII to give a compound of Formula XLVIII;
wherein,
Rₚ is a carboxy protecting group such as methyl, ethyl, allyl, benzyl, t-butyl and silyl; and 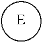
represents mono-, bi- or polycyclic heteroaryl or heterocyclyl selected from the following: 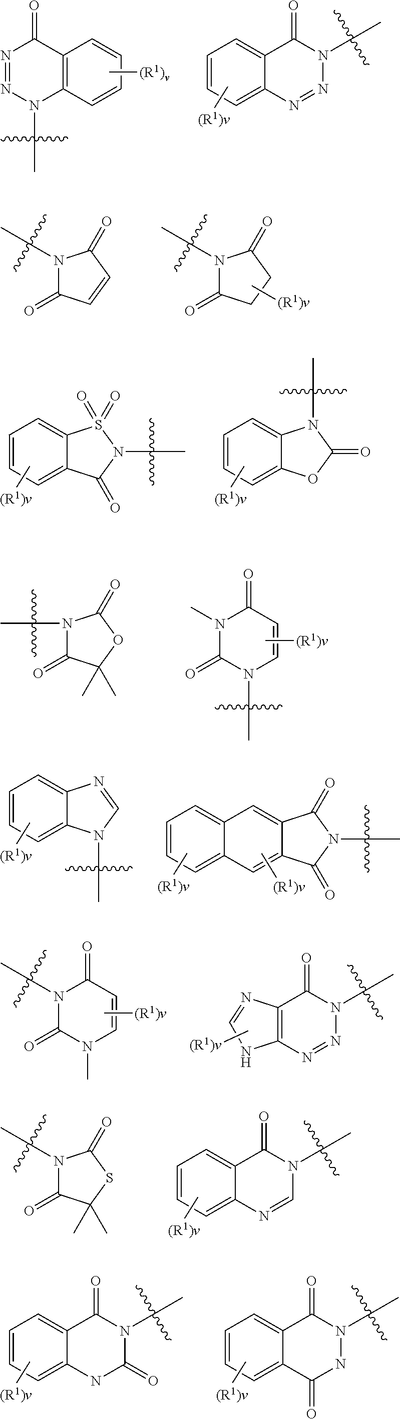 [chemical structure]
